(19) United States
(12) Patent Application Publication
(10) Pub. No.: US 2023/0264006 A1
(43) Pub. Date: Aug. 24, 2023

(54) Delivery Device Apparatuses, Systems, and Methods

(71) Applicant: DEKA Products Limited Partnership, Manchester, NH (US)

(72) Inventors: Dean Kamen, Bedford, NH (US); Lee A. Batchelder, Auburn, NH (US); Eitan C. Zeira, Hollis, NH (US); Linda R. Rios Guevara, Manchester, NH (US); Joshua A. Frye, Manchester, NH (US); Hans E. Johnson, Salem, NH (US)

(21) Appl. No.: 18/087,058

(22) Filed: Dec. 22, 2022

Related U.S. Application Data
(60) Provisional application No. 63/313,182, filed on Feb. 23, 2022.
(60) Provisional application No. 63/405,635, filed on Sep. 12, 2022.

(51) Int. Cl. A61M 37/00

(52) U.S. Cl. CPC A61M 37/0015; A61M 2037/0023; A61M 2037/0061

(57) ABSTRACT

A delivery device for delivery of medical agent to a barrier may comprise a main body including a central region defining a receptacle, a peripheral region defined by a number of petal members, and a set of retention tabs extending into the receptacle. The delivery device may further comprise a reservoir including at least one delivery sharp, a main interior volume, and at least one septum in fluid communication with the main interior volume. The reservoir may be coupled to the main body via the retention tabs. The delivery device may further comprise an adhesive coupled to the main body. The delivery device may further comprise at least one bias member positioned within the receptacle between the reservoir and a wall of the receptacle.

### CROSS-REFERENCE TO RELATED APPLICATIONS

The present application claims the benefit of U.S. Provisional Application Ser. No. 63/405,635, entitled Delivery Device Apparatuses, Systems, and Methods, filed Sep. 12, 2022, Attorney Docket Number 00101.00341.AA928, and also claims the benefit of U.S. Provisional Application Ser. No. 63/313,182, entitled Delivery Device Apparatuses, Systems, and Methods, filed Feb. 23, 2022, Attorney Docket Number 00101.00333.AA808, each of which being incorporated herein in its entirety.

### STATEMENT REGARDING FEDERALLY SPONSORED RESEARCH OR DEVELOPMENT

This invention was made with Government support under Agreement W911NF-17-3-0003, awarded by ACC-APG-RTP. The Government has certain rights in the invention.

### BACKGROUND

### Field of Disclosure

This disclosure relates to medical agent delivery. More specifically, this disclosure relates to dispensers for therapeutic and other medical agents.

### Description of Related Art

Novel pathogens present a variety of public health challenges which are not simple to quickly overcome. From the medical perspective, existing preventive medicine infrastructure has not been and is not well suited to novel pathogens such as SARS, MERS, Zika, and COVID-19. Other pathogens for which herd immunity does not exist (e.g. Ebola), or highly dangerous pathogens which mutate quickly may present similar challenges. Vaccines typically take years to create and once a vaccine does exist, the prospect of rapidly generating billions of doses would almost certainly exceed current vaccine production capabilities. Without vaccination, other preventative measures such as, testing, contact tracing, and personal protective equipment (PPE) are of elevated importance. Again, however, these preventative measures can only provide as much benefit as relevant supply chains allow. Shortages of PPE and testing kits have plagued medical systems in the United States and elsewhere across the globe as they struggle to address the COVID-19 pandemic. In turn, this has hampered the potential to perform effective contact tracing which is already a vast undertaking due to the scale of the COVID-19 pandemic. Additionally, novel pathogens may refocus medical systems away from their typical functions. Secondary impacts often result when the medical community's attention is demanded by a widespread pandemic. This can take the form of delayed surgeries, elective procedures, routine doctor's office visits, etc., but secondary impacts can also be much worse. As has been pointed out by the Chief of Immunizations at UNICEF, for example, during efforts to control an Ebola outbreak in the Democratic Republic of the Congo in 2019 the number of deaths due to measles was double the death toll from Ebola.

Novel pathogens also present challenges that are more psychological in nature. Put simply, such pathogens scare people. Without readily available PPE and testing, people may elect to avoid visiting medical facilities or clinics for fear of exposure to disease. Even with readily available PPE, certain individuals, such as populations in high risk demographics for a particular pathogen, may still have misgivings about visiting such facilities. Additionally, as has been the case in the United States, some may fiercely object to usage of PPE for various reasons. This presents a further public health challenge to systems attempting to deal with pandemics. Solutions to novel pathogens should seek to address and work around these challenges in order to be effective.

### SUMMARY

In accordance with an example embodiment of the present disclosure a method of delivering fluid to a biologic barrier may comprise stretching the biological barrier. The method may further comprise puncturing into the stretched biological barrier with at least one delivery sharp. The method may further comprise displacing the at least one delivery sharp along a non-straight displacement path within the barrier. The method may further comprise dispensing fluid out of the at least one delivery sharp and into the biological barrier.

In some embodiments, puncturing the biological barrier may comprise displacing the at least one delivery sharp in a direction substantially perpendicular to the biological barrier at least until the at least one delivery sharp contacts the biological barrier. In some embodiments, the at least one deliver sharp may comprise at least one microneedle. In some embodiments, the displacement path may be arcuate. In some embodiments, displacing the at least one delivery sharp may result in the delivery sharp being disposed at an orientation closer to a normal orientation with respect to the biological barrier than the orientation of the delivery sharp upon puncturing the biological barrier. In some embodiments, stretching the biological barrier may comprise adhering a delivery device including the at least one delivery sharp to the biological barrier and spreadingly displacing at least two portions of the delivery device. In some embodiments, spreadingly displacing at least two portion of the delivery device may comprise applying a force to the delivery device in a direction normal to the biological barrier. In some embodiments, dispensing fluid out of the at least one delivery sharp may comprise ramping up delivery pressure of fluid supplied to the delivery sharp at least until delivery from the at least one delivery sharp into the biological barrier begins. In some embodiments, displacing the at least one delivery sharp along the non-straight displacement path may comprise driving a beveled edge of the delivery sharp through the barrier. In some embodiments, displacing the at least one delivery sharp along the non-straight displacement path may comprise displacing a face of each of the at least one delivery sharp including an outlet of that delivery sharp away from a portion of the barrier contacted during initial puncture of the barrier.

In accordance with an exemplary embodiment of the present disclosure a method of delivering fluid into a biological barrier may comprise adhering a delivery device including at least one delivery sharp onto the barrier. The method may further comprise applying a force against a portion of the delivery device to displace a reservoir assembly bearing the at least one delivery sharp toward the barrier. The method may further comprise puncturing the barrier with the at least one delivery sharp. The method may further comprise tilting the reservoir assembly and at least one delivery sharp by contacting a member projecting from one side of the reservoir assembly with the barrier while continuing to displace an opposing side of the reservoir assembly toward the barrier. The method may further comprise driving fluid from an interior volume of the reservoir assembly out of the at least one delivery sharp.

In some embodiments, driving fluid from the interior volume of the reservoir assembly may comprise at least partially collapsing the interior volume of the reservoir assembly. In some embodiments, the interior volume of the reservoir assembly may include a first portion and a second portion more proximal the at least one delivery sharp than the first portion. Driving fluid from an interior volume of the reservoir assembly may comprise ramping up pressure of an agent in the second portion of the reservoir at least until delivery from the at least one delivery sharp into the barrier begins. In some embodiments, the at least one delivery sharp may include at least one microneedle. In some embodiments, tilting the reservoir assembly and at least one delivery sharp may comprise driving a beveled edge of the microneedle through the barrier. In some embodiments, the method may further comprise transitioning a bias member of the delivery device to a stressed state and exerting a restoring force of the bias member against the reservoir assembly. In some embodiments, the method may further comprise spreadingly displacing at least two portions of the delivery device.

In accordance with another example embodiment of the present disclosure a method of positioning a delivery sharp for shallow delivery within a biological barrier may comprise stretching the biological barrier. The method may further comprise displacing a holder of the delivery sharp toward the biological barrier. The method may further comprise puncturing the biological barrier with the at least one delivery sharp by displacing the at least one delivery sharp in a direction substantially normal to the biological barrier. The method may further comprise contacting the biological barrier with at least one rocker member protruding from one side of the holder. The method may further comprise continuing displacing a side of the holder opposite the at least one rocker member toward the barrier. The method may further comprise displacing the at least one delivery sharp along a non-straight path within the skin.

In some embodiments, stretching the biological barrier may comprise spreadingly displacing at least two portions of a delivery device including the holder and at least one delivery sharp. In some embodiments, the at least one delivery sharp may be a microneedle. In some embodiments, displacing the at least one delivery sharp along the non-straight path may comprise displacing the delivery sharp along a curved displacement path as the side of the holder opposite the at least one rocker member continues to displace toward the barrier. In some embodiments, displacing the at least one delivery sharp may result in the delivery sharp being disposed at an orientation closer to a normal orientation with respect to the barrier than the orientation of the delivery sharp upon puncturing of the barrier. In some embodiments, displacing the sharp along the non-straight path may comprise swinging the at least one delivery sharp along an arc of between 3-5°.

In accordance with another example embodiment of the present disclosure a fluid delivery assembly may comprise a delivery implement. The delivery implement may comprise a fluid holding portion and a coupling. The delivery assembly may further comprise an adapter. The adapter may comprise an adapter coupling configured to engage with the coupling of the delivery implement to form a fluid tight coupling. The adapter may further comprise an adapter body. The adapter may further comprise a sharp bearing body having chamfered sidewalls and at least one delivery sharp projecting from a face thereof. The adapter body may be molded such that material of the adapter body covers only a portion of the sidewalls to couple the sharp bearing body and adapter body to one another.

In some embodiments, the delivery implement may be a syringe. In some embodiments, the coupling of the delivery implement and the adapter coupling are cooperating luer lock couplings. In some embodiments, the at least one delivery sharp may be a microneedle. In some embodiments, the sharp bearing body and at least one delivery sharp may be formed of silicon. In some embodiments, the at least one delivery sharp may comprise an array of microneedles. In some embodiments, the at least one delivery sharp may include a microneedle having a height of at least 500 microns. In some embodiments, the at least one delivery sharp may include a microneedle having a flow lumen therethrough which communicates with a channel recessed into an exterior face of the microneedle. In some embodiments, the delivery sharp and the at least one delivery sharp may be formed as a monolithic structure.

In accordance with another example embodiment of the present disclosure an adapter for a delivery implement may comprise an adapter coupling configured to engage in fluid tight relationship with a cooperating coupling of the delivery implement. The adapter may further comprise an adapter body. The adapter may further comprise a sharp bearing body having chamfered sidewalls and at least one delivery sharp projecting from a face thereof. The adapter body may be molded to cover only a portion of the sidewalls to capture the sharp bearing body on the adapter.

In some embodiments, the footprint of the sharp bearing body may increase as distance from the face increases. In some embodiments, the sharp bearing body may be formed of silicon. In some embodiments, the sharp bearing body and the at least one delivery sharp may be formed as a single monolithic component. In some embodiments, the at least one delivery sharp may be a microneedle. In some embodiments, the at least one delivery sharp may be a microneedle with a height greater than 500 microns. In some embodiments, the adapter coupling may be a luer lock coupling. In some embodiments, the at least one delivery sharp may include an array of microneedles. In some embodiments, the at least one delivery sharp may include a microneedle having a flow lumen therethrough which communicates with a channel recessed into an exterior face of the microneedle.

In accordance with another example embodiment of the present disclosure a delivery device may comprise a main body including a central region and a peripheral region. The peripheral region may have a plurality of petal members extending in an outward direction from the central region. The delivery device may further comprise a variable volume reservoir coupled to the main body. The delivery device may further comprise a sharp bearing body having at least one microneedle projecting from a face thereof and having sidewalls oriented at an acute angle to the face. Material of a rigid portion of the reservoir may be molded over only a portion of the sidewalls to couple the sharp bearing body and reservoir. The delivery device may further comprise an adhesive disposed on at least a portion of the main body.

In some embodiments, the central region may define a receptacle and may include a top region and a base which are connected by a wall. The petal members may extend from the base. In some embodiments, the reservoir may be at least partially disposed within the receptacle. In some embodiments, the delivery device may further comprise a bias member disposed within the receptacle between the top region and the reservoir. In some embodiments, the sharp bearing body and at least one microneedle may be a single monolithic structure. In some embodiments, the sharp bearing body and at least one microneedle may be formed of silicon. In some embodiments, the reservoir may include a first portion and a second portion separated from one another via a flow restrictor. The second portion may be more proximal the at least one microneedle than the first portion.

In accordance with still another exemplary embodiment of the present disclosure a method of forming a microneedle bearing body may comprise etching a plurality of microneedles on a silicon wafer. The method may further comprise dicing a sharp bearing body including at least one of the microneedles from the wafer. The dicing may form sidewalls of the sharp bearing body angled such that the footprint of the sharp bearing body increases as distance from a face on which that at least one microneedle is disposed increases. The method may further comprise molding a body over only a portion of the sidewalls of the sharp bearing body distal to the face.

In some embodiments, the body may be a portion of a syringe. In some embodiments, the body may be an adapter for attachment to a delivery implement. In some embodiments, the body may form a portion of a delivery device. In some embodiments, the body may be a stage projection of a reservoir of a delivery device. In some embodiments, the plurality of microneedles may each include a flow lumen in communication with a channel recessed into an exterior face of the microneedle. In some embodiments, the sharp bearing body may include an array of microneedles. In some embodiments, the sharp bearing body may include a row of microneedles. In some embodiments, dicing the sharp bearing body from the wafer may comprise using a dicing saw with a blade configured to form the angled side walls. In some embodiments, etching the plurality of microneedles comprises etching the plurality of microneedles to have a height greater than 500 microns.

In accordance with another example embodiment of the present disclosure a delivery device package may comprise a first portion formed of a rigid material and including a plurality of wells, at least two of the wells connected by a passage. The package may further comprise a second portion removably coupled to a rim of the first portion. The package may further comprise a delivery device having a main body bearing an adhesive covered by an adhesive liner. The adhesive liner may be coupled to a surface of a first well. When the main body is displaced out of the well, the adhesive liner may be configured to peel off the adhesive and remain in the well.

In some embodiments, at least one of the first and second portion may include a unique identifier. In some embodiments, the second portion may be permeable to a sterilizing agent. In some embodiments, the delivery device may include a pull tab which extends from the main body through the passage and into a second well. In some embodiments, the delivery device may include at least one delivery sharp and a delivery sharp cap surrounding the at least one delivery sharp. In some embodiments, the cap may be coupled to the surface of the first well and configured to remain in the well when the delivery device is displaced out of the well.

In accordance with still another example embodiment of the present disclosure a delivery device for delivering fluid into a biological barrier may comprise a main body. The delivery device may further comprise an adhesive on at least a portion of the main body for adhering the delivery device to the biological barrier. The delivery device may further comprise a reservoir coupled to the main body including at least one delivery sharp and at least one rocker member. The main body may be configured to transition between a first state and a second state. The reservoir may be configured to displace toward the biological barrier as the main body transitions from the first state to the second state and the rocker member may be configured to tilt the reservoir after the reservoir has displaced a distance toward the biological barrier.

In some embodiments, the reservoir may be at least partially collapsible. In some embodiments, at least two section of the main body on which the adhesive is disposed may be configured to spreadingly displace as the main body transitions from the first state to the second state. In some embodiments, the main body may include a central region and a peripheral region having a plurality of petal members extending outwardly form the central region. The central region may define a receptacle for at least a portion of the reservoir. In some embodiments, the reservoir may include at least one rigid portion and at least one flexible portion. In some embodiments, the at least one rocker member may be disposed along a peripheral edge of a rigid portion of the reservoir. In some embodiments, the at least one rocker member may be a protuberance extending from a rigid portion of the reservoir. In some embodiments, the at least one delivery sharp may be mounted to a stage projecting from a rigid portion of the reservoir. The at least one rocker member may be disposed more proximal the periphery of the rigid portion than the stage. In some embodiments, the at least one rocker member may have a height substantially even with the height of the stage. In some embodiments, the at least one delivery sharp may include at least one microneedle and the portion of the at least one microneedle most proximal the at least one rocker member may be a cutting edge of the microneedle. In some embodiments, the at least one rocker member may be configured to tilt the reservoir such that the at least one delivery sharp swings along an arc of between 3-5°. In some embodiments, the at least one rocker member may be configured to tilt the reservoir only after the biological barrier has been punctured by the at least one delivery sharp.

In accordance with yet another example embodiment of the present disclosure a delivery device may comprise a main body including a central region and a peripheral region having a plurality of petal members extending from the central region. The central region may define a receptacle and may have a top region and a base which may be connected by a wall. The delivery device may further comprise a variable volume reservoir including at least one delivery sharp and at least one rocker member more proximal the periphery of the reservoir than the at least one delivery sharp. The reservoir may be coupled to the main body and at least partially disposed within the receptacle. The delivery device may further comprise an adhesive disposed on at least a portion of the main body.

In some embodiments, the at least one delivery sharp may be a microneedle. In some embodiments, the rocker member may be a protuberance extending from a rigid portion of the reservoir. In some embodiments, the at least one delivery sharp may include a microneedle and the portion of the microneedle most proximal the rocker member may be a cutting edge of the microneedle. In some embodiments, the rocker member may be disposed at a peripheral edge of the reservoir. In some embodiments, the reservoir may include a planar body including a stage projecting therefrom. The at least one delivery sharp may be mounted to the stage. In some embodiments, the delivery sharp may project from the stage at an angle which is acute to a plane perpendicular to the plane of the planar body. In some embodiments, the rocker member may bear a marking agent. In some embodiments, the main body may be configured to transition from a first state to a second state when adhered to a surface via the adhesive. The reservoir may be configured to displace in a direction substantially normal to the surface until the rocker member contacts the surface as the main body transitions from the first state to the second state. A portion of the reservoir opposite the rocker member may be configured to continue to displace toward the surface once the rocker member has contacted to the surface as the main body transitions from the first state to the second state. In some embodiments, the at least one delivery sharp may be configured to puncture the surface as the reservoir displaces in the direction substantially normal to the surface and subsequently tilt as the a portion of the reservoir opposite the rocker member continues to displace toward the surface once the rocker member has contacted to the surface.

In accordance with another example embodiment of the present disclosure a delivery device for delivering fluid into a biological barrier may comprise a main body. The delivery device may further comprise an adhesive on at least a portion of the main body for adhering the delivery device to the biological barrier. The delivery device may further comprise a reservoir coupled to the main body including at least one delivery sharp. The reservoir may include a main interior volume for holding fluid and a septum in fluid communication with the main interior volume. The main body may be configured to transition between a first state and a second state. The reservoir may be configured to displace toward the biological barrier as the main body transitions from the first state to the second state.

In some embodiments, the reservoir may further comprise a protuberance disposed more proximal a periphery of the reservoir than the at least one delivery sharp. In some embodiments, the protuberance may be configured to tilt the reservoir as the main body transitions from the first state to the second state. In some embodiments, the main interior volume may be at least partially collapsible. In some embodiments, at least two section of the main body on which the adhesive is disposed may be configured to spreadingly displace as the main body transitions from the first state to the second state. In some embodiments, the main body may include a central region and a peripheral region having a plurality of petal members extending outwardly from the central region. The central region may define a receptacle for at least a portion of the reservoir. In some embodiments, the reservoir may include at least one rigid portion and at least one flexible portion. In some embodiments, the reservoir may include a guard for protecting a portion of the reservoir from contacting a dispensing sharp which has pierced the septum. In some embodiments, the guard may be defined at least partially by the septum and establish a sharp receiving volume within the reservoir. In some embodiments, the reservoir may include a flow path extending from the main interior volume to the septum. In some embodiments, the septum may include a plug portion disposed within an aperture in a rigid portion of the reservoir. In some embodiments, the septum may include a plug portion and a standoff extending from the plug portion. The standoff may include two raised portions flanking a recessed channel. In some embodiments, the at least one delivery sharp may be mounted to a stage projecting from a rigid portion of the reservoir. In some embodiments, the at least one delivery sharp may include at least one microneedle. In some embodiments, the reservoir may further comprise at least one marking agent disposed on a raised portion of the reservoir. In some embodiments, the reservoir may further comprises a marking member.

In accordance with still another example embodiment of the present disclosure a delivery device may comprise a main body including a central region and a peripheral region having a plurality of petal members extending from the central region. The central region may define a receptacle and have a top region and a base which are connected by a wall. The delivery device may further comprise a variable volume reservoir including at least one delivery sharp and at least one septum in communication with a main interior volume of the reservoir. The reservoir may be coupled to the main body and at least partially disposed within the receptacle. The delivery device may further comprise an adhesive disposed on at least a portion of the main body.

In some embodiments, the reservoir may include at least one projecting member more proximal the periphery of the reservoir than the at least one delivery sharp. In some embodiments, the projecting member may be extend from a rigid portion of the reservoir. In some embodiments, the at least one delivery sharp may be a microneedle. In some embodiments, the reservoir may include a planar body including a stage projecting therefrom. The at least one delivery sharp may be mounted to the stage. In some embodiments, the delivery sharp may project from the stage at an angle which is acute to a plane perpendicular to the plane of the planar body. In some embodiments, when adhered to a surface via the adhesive, the main body may be configured to transition from a first state to a second state. The reservoir may be configured to displace primarily in a direction substantially normal to the surface as the main body transitions from the first state to the second state. In some embodiments, the reservoir may include a guard. In some embodiments, the guard may be defined at least partially by the septum. In some embodiments, a fluid pathway form the main interior volume to the septum may be included in the reservoir. In some embodiments, the septum may include a plug portion forming a fluid tight seal against an aperture in a rigid portion of the reservoir. In some embodiments, the septum may include a first portion and a second portion extending from the first portion. The second portion may include two raised portions flanking a recessed channel. In some embodiments, the reservoir may further comprise a marking member. In some embodiments, the marking agent may be borne on the marking member and the marking member and the at least one delivery sharp may extend in respective directions away from a same side of the reservoir.

In accordance with another example embodiment of the present disclosure a delivery device for delivery of medical agent to a biological barrier may comprise a main body including a central region defining a receptacle, a peripheral region defined by a number of petal members, and a set of retention tabs extending into the receptacle. The delivery device may further comprise a reservoir including at least one delivery sharp, a main interior volume, and at least one septum in fluid communication with the main interior volume. The reservoir may be coupled to the main body via the retention tabs. The delivery device may further comprise an adhesive coupled to the main body. The delivery device may further comprise at least one bias member positioned within the receptacle between the reservoir and a wall of the receptacle.

In some embodiments, the at least one bias member may be a coil spring having an integral reservoir interfacing member. In some embodiments, the main body may be configured to transition between a first state and a second state. The reservoir may be configured to displace toward the biological barrier as the main body transitions from the first state to the second state. In some embodiments, the bias member may be configured to distort upon transition of the main body from the first state to the second state. The bias member configured to restore against the reservoir urging fluid out of the at least one delivery sharp once distorted by transition of the main body from the first to the second state. In some embodiments, the bias member may be select from a list consisting of a foam body, a rubber body, and an elastomeric body. In some embodiments, the main interior volume may be formed by a wall including at least one collapse facilitator. In some embodiments, the at least one bias member is formed of an elastomeric material and includes at least one hollow portion. In some embodiments, the at least one hollow portion may be formed by a passage extending through the bias member. In some embodiments, the bias member may be formed of an elastomeric material and may have at least a first and second tier having footprints with differing areas. In some embodiments, the reservoir may include a sharp receiving volume. In some embodiments, the reservoir may comprise a guard defined at least partially by a septum. In some embodiments, a fluid pathway from the main interior volume to a septum may be included in the reservoir. In some embodiments, the device may further comprise a septum and the septum may include a plug portion seated in fluid tight relationship within an aperture through a rigid portion of the reservoir. In some embodiments, the device may further comprise a septum, the septum may include a portion disposed within a fluid containing portion of the reservoir and that portion may include two raised sections flanking a recessed channel. In some embodiments, the reservoir may include a rigid portion and a flexible portion coupled to the rigid portion. The delivery device may further comprise a septum, the septum may include a first section disposed within a passage through the rigid portion. The septum may include a second section having recessed channel and being configured to block displacement of the flexible portion into the recessed channel. In some embodiments, the reservoir may be filled with an overdose intervention drug. In some embodiments, the reservoir may be filled with at least one vaccine. In some embodiments, the reservoir may be filled with a COVID-19 vaccine. In some embodiments, the reservoir may further comprise a projection more proximal a periphery of the reservoir than the at least one delivery sharp. In some embodiments, the main body may be configured to transition between a first state and a second state. The reservoir may be configured to displace toward the biological barrier as the main body transitions from the first state to the second state. The projection may be configured to tilt the reservoir as the main body transitions from the first state to the second state. In some embodiments, the main body may be formed integrally with the retention tabs in a side action free injection molding operation. In some embodiments, the main body may include a set of stop members associated with the retention tabs.

In accordance with yet another exemplary embodiment of the present disclosure a delivery device may comprise a main body including a central region and a peripheral region having a plurality of petal members extending from the central region. The central region may define a receptacle and have a top region and a base which are connected by a wall. The delivery device may further comprise a variable volume reservoir including at least one delivery sharp. The reservoir may be coupled to the main body and at least partially disposed within the receptacle. The delivery device may further comprise an elastomer body within the receptacle between the top region and reservoir. The delivery device may further comprise an adhesive disposed on at least a portion of the main body.

In some embodiments, the elastomer body may include at least one hollow portion. In some embodiments, each of the at least one hollow portion may be formed by a passage extending at least partially through the elastomer body. In some embodiments, the elastomer body may be tiered. In some embodiments, the reservoir further may include a septum. In some embodiments, the main body may include a set of retention tabs extending into the receptacle. The reservoir may be configured to be coupled to the reservoir by snap fit via advancement into and beyond the retention tabs. In some embodiments, the reservoir may further comprise a protrusion more proximal a periphery of the reservoir than the at least one delivery sharp. In some embodiments, a marking agent may be borne on the protrusion. In some embodiments, the main body may be configured to transition between a first state and a second state. The reservoir may be configured to displace along a path as the main body transitions from the first state to the second state. In some embodiments, the at least one delivery sharp may include a spaced array of microneedles. In some embodiments, the majority of each of the petal members is flat and positioned at an acute angle to a center axis of the delivery device. In some embodiments, the peripheral region may include an outermost portion defining a peripheral edge of the main body. Each of the petal members may define a portion of the peripheral edge. In some embodiments, an outermost portion of each of the petal members may define a portion of the peripheral edge of the main body. The outermost portion of each of the petal members may be arched along a radius having a center point along the center axis of the delivery device. In some embodiments, an outermost portion of each of the petal members may define a portion of the peripheral edge of the main body. The outermost portion of each of the petal members may be arched along a radius having a center point within the respective petal member. In some embodiments, each petal member may be separated by slits in the peripheral region. Each of the petal members may include an outermost region disposed more distal the central region than the ends of the slits most distal the central region. The outermost regions may decrease in width as distance from the central region increases.

In accordance with another example embodiment of the present disclosure a method of delivering agent to an injection site may comprise adhering a delivery device to a patch of skin at the injection site. The method may further comprise distorting at least two portions of the delivery device adhered to the patch of skin from an initial state to a distorted state such that the patch of skin is stretched and at least one delivery sharp of the delivery device is displaced into a puncturing relationship with the patch of skin. The method may further comprise dispensing fluid from a reservoir of the delivery device into the injection site via the at least one delivery sharp. The method may further comprise restoring, the at least two portions of the delivery device adhered to the patch of skin from the distorted state, toward the initial state. The method may further comprise removing the delivery device from the patch of skin.

In some embodiments, the at least one delivery sharp may be a microneedle. In some embodiments, the at least one delivery sharp may be a spaced array of microneedles. In some embodiments, the method further may comprise tilting the at least one delivery sharp while the delivery sharp is in a puncturing relationship with the patch of skin. In some embodiments, the method may further comprise compressing a bias member of the delivery device against the reservoir. In some embodiments, distorting the at least two portions of the delivery device adhered to the patch of skin may comprise applying pressure to a surface of the delivery device. In some embodiments, restoring the at least two portions of the delivery device adhered to the patch of skin from the distorted state, toward the initial state may comprise relieving pressure applied to a surface of the delivery device. In some embodiments, dispensing fluid from the reservoir may comprise substantially inverting a section of the delivery device so as to compress a bias member intermediate the section and the reservoir. In some embodiments, dispensing fluid from the reservoir may comprise transitioning a section of the delivery device from a protruding orientation to an indented orientation so as to compress a bias member intermediate the section and the reservoir. In some embodiments, the at least two portions of the delivery device may be petal members of a main body of the delivery device which may be coupled to a central region of the main body. In some embodiments, dispensing fluid from the reservoir may occur at least partially while the at least two portions of the delivery device restore toward the initial state. In some embodiments, dispensing of fluid from the reservoir may continue after the at least two portion sof the delivery device restore toward the initial state.

In accordance with another example embodiment of the present disclosure a method of delivering an agent may comprise affixing to a surface a delivery device including at least one delivery sharp coupled to a collapsible reservoir containing the agent while the delivery device is in a first state. The method may further comprise applying and relieving pressure on the delivery device to transition the delivery device to a second state, penetrate the surface with the at least one delivery sharp, and cause collapse of the reservoir to force the agent out of the at least one delivery sharp.

In some embodiments, the method may further comprise stretching the skin as the delivery device transitions from the first state to the second state. In some embodiments, the method may further comprise tilting the at least one delivery sharp after the delivery sharp has penetrated the surface. In some embodiments, the method may further comprise contacting the surface with a protrusion from the reservoir after the delivery sharp has penetrated the surface. In some embodiments, the method may further comprise creating a mark on the surface with the protrusion. In some embodiments, the method may further comprise filling the reservoir through a septum. In some embodiments, the at least one delivery sharp may be a microneedle. In some embodiments, the at least one delivery sharp may be a spaced array of microneedles. In some embodiments, the method may further comprise compressing a bias member against the reservoir. In some embodiments, the method may further comprise compressing an elastomeric body against the reservoir. In some embodiments, the method may further comprise distorting the at least two portions of the delivery device affixed to the surface from an initial state to a spreadingly displaced state. In some embodiments, applying and relieving pressure to the delivery device to transition the delivery device to a second state may comprise distorting at least two portions of the delivery device affixed to the skin from a storage state through an intermediate state and at least partially back toward the storage state. In some embodiments, applying and relieving pressure to the delivery device to transition the delivery device to a second state may comprise distorting at least two portions of the delivery device affixed to the skin from a storage state to a maximally distorted state and allowing the at least two portions to at least partially elastically restore from the maximally distorted state. In some embodiments, the at least two portions may be petal members extending peripherally from a central region of a main body of the delivery device. In some embodiments, dispensing fluid from the reservoir may comprise substantially inverting a section of the delivery device so as to compress a bias member intermediate the section and the reservoir.

In accordance with another example embodiment of the present disclosure a method of delivering an agent may comprise affixing to a surface a delivery device including at least one delivery sharp coupled to a collapsible reservoir containing the agent. The method may further comprise applying pressure on the delivery device to transition first and second portions of the delivery device to respective distorted states, penetrate the surface with the at least one delivery sharp, and initiate collapse of the reservoir to force the agent out of the at least one delivery sharp. The method may further comprise relieving pressure on the delivery device to allow the first portion of the delivery device to at least partially restore from its distorted state and lift the surface.

In some embodiments, the transitioning the first portion to the distorted state may comprise spreadingly displacing segments of the first portion with respect to one another. In some embodiments, the method may further comprise tilting the at least one delivery sharp after the delivery sharp has penetrated into the surface. In some embodiments, the method may further comprise contacting the surface with a protrusion from the reservoir after the at least one delivery sharp has penetrated into the surface. In some embodiments, the collapse of the reservoir may continue while the first portion of the delivery device at least partially restores from its distorted state. In some embodiments, the at least one delivery sharp may be a microneedle. In some embodiments, the at least one delivery sharp may be a spaced array of microneedles. In some embodiments, transitioning the second portion to its distorted state may comprise compressing a bias member against the reservoir. In some embodiments, transitioning the second portion to its distorted state may comprise transitioning the second portion from a protruding state to an indented state. In some embodiments, the first portion of the delivery device may comprises a plurality of petal members extending peripherally from a central region of a main body of the delivery device and the second portion of the delivery device may be a section of the central region.

In accordance with still another example embodiment of the present disclosure a delivery device for delivering fluid into a biological barrier may comprise a main body including a peripheral region with a plurality of petal members. The delivery device may further comprise a reservoir coupled to the main body including at least one delivery sharp. The main body may be configured to transition between a first state and a second state. The petal members may be configured to distort from an initial state, to an intermediate state, and at least partially restore from the intermediate state as the main body transitions from the first state to the second state.

In some embodiments, the delivery device may further comprise an adhesive on at least a portion of the main body for adhering the delivery device to the biological barrier. In some embodiments, the reservoir may further comprise at least one rocker member. In some embodiments, the reservoir may be configured to displace along a displacement path as the main body transitions from the first state to the second state. In some embodiments, the reservoir may include a rigid portion having at least one protuberance. The protuberance may be configured to engender tilting of the reservoir as the reservoir displaces in along the displacement path. In some embodiments, the reservoir may be at least partially collapsible. In some embodiments, the petal members may be at least partially covered with adhesive. The petal members configured to spreadingly displace as the petal members distort from the initial state to the intermediate state. In some embodiments, the main body may include a central region. The plurality of petal members may extend in a direction outwardly form the central region. The central region may define a receptacle for at least a portion of the reservoir. In some embodiments, the reservoir may include at least one rigid portion and at least one flexible portion. In some embodiments, the flexible region may include a reservoir wall with a collapse facilitator. In some embodiments, the majority of each petal member may be substantially flat. In some embodiments, the majority of each petal member may extend at a constant angle to a center axis of the delivery device and each petal member may include a segment having a curvature leading to a central region of the main body. In some embodiments, each petal member may include a curved segment intermediate a first substantially flat region and second substantially flat region of each petal member. In some embodiments, the at least one delivery sharp may be mounted to a stage projecting from a rigid section of the reservoir. In some embodiments, the at least one delivery sharp may include at least one microneedle. In some embodiments, the peripheral edge of the delivery device may be defined by the outermost edges of the petal members. The outermost edges may be curved along a path other than that defined by a constant radius extending from a point along the center axis of the delivery device. In some embodiments, each petal member may include an outermost region having a width which decreases as distance from a central region of the delivery device increases. Each outermost region may include an outermost edge along a path other than that defined by a constant radius extending from a point along the center axis of the delivery device. In some embodiments, the main body may include a central region formed integrally with the peripheral region. The central region may include a portion configured to flip from a protruding state to a depressed state as the delivery device transitions from the first state to the second state. In some embodiments, the delivery device may further comprise a bias member between the reservoir and the portion. The bias member may be configured to distort when the portion is flipped to the depressed state. In some embodiments, the bias member may be elastomer. In some embodiments, the bias member may be a tiered elastomeric body having at least one passage extending at least partially therethrough. In some embodiments, the reservoir may further comprise a septum.

### BRIEF DESCRIPTION OF THE DRAWINGS

These and other aspects will become more apparent from the following detailed description of the various embodiments of the present disclosure with reference to the drawings wherein:

FIG. 1A is a block diagram of an example delivery device in a storage state in accordance with various aspects and embodiments of the present disclosure;

FIG. 1B is a block diagram of an example delivery device in a delivery state in accordance with various aspects and embodiments of the present disclosure;

FIG. 2 is a diagram of an example microneedle in accordance with various aspects and embodiments of the present disclosure;

FIG. 3A is a diagram of an example sharp bearing body incorporating microneedles in accordance with various aspects and embodiments of the present disclosure;

FIG. 3B is a diagram of an example microneedle in accordance with various aspects and embodiments of the present disclosure;

FIG. 4A is a diagram of an example sharp bearing body incorporating microneedles in accordance with various aspects and embodiments of the present disclosure;

FIG. 4B is a diagram of an example microneedle in accordance with various aspects and embodiments of the present disclosure;

FIG. 5A is a perspective view of an example sharp bearing body including a set of exemplary microneedles in accordance with various aspects and embodiments of the present disclosure;

FIG. 5B is a perspective view of an example sharp bearing body including a set of exemplary microneedles in accordance with various aspects and embodiments of the present disclosure;

FIG. 6A is a perspective view of an example sharp bearing body include a set of exemplary microneedles in accordance with various aspects and embodiments of the present disclosure;

FIG. 6B is a top plan view of the example sharp bearing body shown in FIG. 6A in accordance with various aspects and embodiments of the present disclosure;

FIG. 7A is a top down view of an example sharp bearing body including a set of exemplary microneedles in accordance with various aspects and embodiments of the present disclosure;

FIG. 7B is a perspective view of an example sharp bearing body including a set of exemplary microneedles in accordance with various aspects and embodiments of the present disclosure;

FIG. 8A is a top down view of an example sharp bearing body including a set of microneedles in accordance with various aspects and embodiments of the present disclosure;

FIG. 8B is a perspective view of an example sharp bearing body including a set of exemplary microneedles in accordance with various aspects and embodiments of the present disclosure;

FIG. 8C is a cross-sectional view taken at the indicated cut plane of FIG. 8A in accordance with various aspects and embodiments of the present disclosure;

FIGS. 9A-9D depict various views of an exemplary microneedle with side ports in accordance with various aspects and embodiments of the present disclosure;

FIG. 10A is a block diagram of parts of an example delivery device in accordance with various aspects and embodiments of the present disclosure;

FIG. 10B is a block diagram of parts of an example delivery device in accordance with various aspects and embodiments of the present disclosure;

FIG. 11 is a block diagram of parts of an example delivery device in accordance with various aspects and embodiments of the present disclosure;

FIG. 12 is a perspective view diagram of an example delivery device in accordance with various aspects and embodiments of the present disclosure;

FIG. 13 is a plan view diagram of an example delivery device in accordance with various aspects and embodiments of the present disclosure;

FIG. 14 is a side view diagram of an example delivery device in accordance with various aspects and embodiments of the present disclosure;

FIG. 15 is a plan view diagram of an example delivery device in accordance with various aspects and embodiments of the present disclosure;

FIG. 16 is a conceptual representation of an exemplary delivery device in a delivery state in accordance with various aspects and embodiments of the present disclosure;

FIG. 17 is a diagram of an example delivery device in a storage state in accordance with various aspects and embodiments of the present disclosure;

FIG. 18 is a conceptual representation of an example delivery device in a delivery state in accordance with various aspects and embodiments of the present disclosure;

FIG. 19 is a side view diagram of an example delivery device in accordance with various aspects and embodiments of the present disclosure;

FIG. 20 is a perspective view diagram of an example delivery device in accordance with various aspects and embodiments of the present disclosure;

FIGS. 21A-21I depicts various example embodiments of main bodies including different slot patterns and top surface apertures in accordance with various aspects and embodiments of the present disclosure;

FIG. 22 is a perspective view diagram of an example delivery device in accordance with various aspects and embodiments of the present disclosure;

FIG. 23 is a perspective view diagram of an example delivery device in accordance with various aspects and embodiments of the present disclosure;

FIG. 24 is a perspective view diagram of an example delivery device in accordance with various aspects and embodiments of the present disclosure;

FIG. 25 depicts a perspective view of an example delivery device package in accordance with various aspects and embodiments of the present disclosure;

FIG. 26 depicts a top plan view of an example delivery device package with the delivery device removed in accordance with various aspects and embodiments of the present disclosure;

FIG. 27 is a diagram of an example delivery device in a storage state in accordance with various aspects and embodiments of the present disclosure;

FIG. 28 is a cross-section perspective view of a main body of an example delivery device in a storage state in accordance with various aspects and embodiments of the present disclosure;

FIG. 29A is a plan view diagram of an example delivery device illustrating example dimensions of one delivery device embodiment in accordance with an embodiment of the present disclosure;

FIG. 29B is a side view diagram of an exemplary delivery device illustrating example dimensions of one delivery device embodiment in accordance with an embodiment of the present disclosure;

FIG. 30A is a side view conceptual diagram of an example delivery device in transition to a delivery state in accordance with various aspects and embodiments of the present disclosure;

FIG. 30B is a side view conceptual diagram of an example delivery device in transition to a delivery state in accordance with various aspects and embodiments of the present disclosure;

FIG. 31 is a perspective view diagram conceptually illustrating an example delivery device in a delivery state in accordance with various aspects and embodiments of the present disclosure;

FIG. 32 is a side view diagram of the example delivery device of FIG. 31 in accordance with various aspects and embodiments of the present disclosure;

FIG. 33 is an exploded view diagram of the delivery device shown in FIG. 31 in accordance with various aspects and embodiments of the present disclosure;

FIG. 34 is a plan view diagram of the delivery device shown in FIG. 31 in accordance with various aspects and embodiments of the present disclosure;

FIG. 35 is a cross section view diagram of the delivery device shown in FIG. 31 taken at the indicated cut plane in FIG. 34 in accordance with various aspects and embodiments of the present disclosure;

FIG. 36 depicts a representational illustration of an example delivery device including a dispensing assembly in accordance with various aspects and embodiments of the present disclosure;

FIG. 37 depicts a representational illustration of an example delivery device including a dispensing assembly in accordance with various aspects and embodiments of the present disclosure;

FIG. 38 depicts a representational illustration of an example delivery device including a dispensing assembly in accordance with various aspects and embodiments of the present disclosure;

FIG. 39 depicts a perspective view of an example delivery device and bias member in accordance with various aspects and embodiments of the present disclosure;

FIG. 40 depicts a perspective view of an example bias member which may be included in a delivery device in accordance with various aspects and embodiments of the present disclosure;

FIG. 41 depicts a cross-sectional view of a portion of an example delivery device in accordance with various aspects and embodiments of the present disclosure;

FIG. 42 depicts a cross-sectional view of a portion of an example delivery device in accordance with various aspects and embodiments of the present disclosure;

FIG. 43 depicts a perspective view of an example depressor body which may be included in a delivery device in accordance with various aspects and embodiments of the present disclosure;

FIG. 44 depicts an example depressor body and bias member where the bias member is in a stressed state in accordance with various aspects and embodiments of the present disclosure;

FIG. 45 depicts a cross-sectional view of an example depressor body and bias member where the bias member is in a stressed state in accordance with various aspects and embodiments of the present disclosure;

FIG. 46A depicts a perspective view of an exemplary delivery device in accordance with various aspects and embodiments of the present disclosure;

FIG. 46B depicts a perspective view of an exemplary stop member which may be included in a delivery device in accordance with various aspects and embodiments of the present disclosure;

FIG. 46C depicts a perspective view of an example delivery assembly and example stop member which may be included in a delivery device in accordance with various aspects and embodiments of the present disclosure;

FIG. 46D depicts a perspective view of an example bias member and example depressor body which may be included in a delivery device in accordance with various aspects and embodiments of the present disclosure;

FIG. 47 depicts a representational illustration of an example delivery device including a bias member in accordance with various aspects and embodiments of the present disclosure;

FIG. 48A depicts a bottom plan view of an exemplary main body which may be included in a delivery device in accordance with various aspects and embodiments of the present disclosure;

FIG. 48B depicts a perspective view of an example main body and example bias member which may be included in a delivery device in accordance with various aspects and embodiments of the present disclosure;

FIG. 48C depicts a perspective view of an example main body and example bias member which may be included in a delivery device in accordance with various aspects and embodiments of the present disclosure;

FIG. 49A depicts a side view of an example delivery device in accordance with various aspects and embodiments of the present disclosure;

FIG. 49B depicts a cross-section view of an example delivery device in accordance with various aspects and embodiments of the present disclosure;

FIG. 50 depicts an exploded view of an example delivery device in accordance with various aspects and embodiments of the present disclosure;

FIGS. 51A-51B depict views of portions of an example delivery device respectively in a storage state and a delivery state in accordance with various aspects and embodiments of the present disclosure;

FIG. 52 depicts a bottom view of an example main body of a delivery device and a portion of a depressor body in accordance with various aspects and embodiments of the present disclosure;

FIG. 53A is a perspective view diagram of an example holder for a sharp bearing body in accordance with various aspects and embodiments of the present disclosure;

FIG. 53B is a side view diagram of an example holder for a sharp bearing body in accordance with various aspects and embodiments of the present disclosure;

FIG. 53C is a bottom-up plan view diagram of an example holder for a sharp bearing body in accordance with various aspects and embodiments of the present disclosure;

FIG. 53D is a perspective view diagram of an exemplary holder for a sharp bearing body in accordance with various aspects and embodiments of the present disclosure;

FIG. 54A depicts a perspective view of an example holder including a stage projection in accordance with various aspects and embodiments of the present disclosure;

FIG. 54B depicts a perspective view of an example holder including a stage projection in accordance with various aspects and embodiments of the present disclosure;

FIG. 54C depicts a bottom plan view of an example holder including a stage projection in accordance with various aspects and embodiments of the present disclosure;

FIG. 55A depicts a side view of an example holder including a stage projection to which an example sharp bearing body is mounted in accordance with various aspects and embodiments of the present disclosure;

FIG. 55B depicts a detailed view of the indicated region of FIG. 55A in accordance with various aspects and embodiments of the present disclosure;

FIG. 55C depicts a cross-sectional view of an example holder including a stage projection to which an example sharp bearing body is mounted in accordance with various aspects and embodiments of the present disclosure;

FIG. 55D depicts a detailed view of the indicated region of FIG. 55C in accordance with various aspects and embodiments of the present disclosure;

FIG. 56A is a perspective view diagram of an exemplary portion of a reservoir in accordance with various aspects and embodiments of the present disclosure

FIG. 56B is a side view diagram of an exemplary portion of a reservoir in accordance with various aspects and embodiments of the present disclosure;

FIG. 56C is a perspective view diagram of an exemplary portion of a reservoir in accordance with various aspects and embodiments of the present disclosure;

FIG. 56D is a plan view diagram of an exemplary portion of a reservoir in accordance with various aspects and embodiments of the present disclosure;

FIG. 57 depicts a perspective view of an exemplary reservoir in accordance with various aspects and embodiments of the present disclosure;

FIG. 58 depicts a perspective view of another exemplary reservoir in accordance with various aspects and embodiments of the present disclosure;

FIG. 59 is a block diagram of an example reservoir assembly in accordance with various aspects and embodiments of the present disclosure;

FIG. 60 is a block diagram of an example delivery device in accordance with various aspects and embodiments of the present disclosure;

FIG. 61A depicts a block diagram of an example reservoir assembly in accordance with various aspects and embodiments of the present disclosure;

FIG. 61B depicts a block diagram of an example reservoir assembly in accordance with various aspects and embodiments of the present disclosure;

FIG. 62 depicts a representational illustration of an example delivery device including a reservoir partitioned into a plurality of portions in accordance with various aspects and embodiments of the present disclosure;

FIG. 63A depicts a block diagram view of an example delivery device with an example rocker member in accordance with various aspects and embodiments of the present disclosure;

FIG. 63B depicts a block diagram view of an example delivery device with an example rocker member in accordance with various aspects and embodiments of the present disclosure;

FIG. 64A depicts a perspective view of an example delivery device including an example rocker member in accordance with various aspects and embodiments of the present disclosure;

FIG. 64B depicts a perspective view of another example delivery device including a plurality of example rocker members in accordance with various aspects and embodiments of the present disclosure;

FIG. 65 depicts a flowchart detailing a number of example actions which may be executed to delivery agent with a delivery device in accordance with various aspects and embodiments of the present disclosure;

FIG. 66 depicts an illustration of an example delivery device after being applied to a user in accordance with various aspects and embodiments of the present disclosure;

FIG. 67 depicts an illustration of an example delivery device in process of transitioning from a storage state to a delivery state in accordance with various aspects and embodiments of the present disclosure;

FIG. 68 depicts an illustration of an example delivery device in process of transitioning from a storage state to a delivery state in accordance with various aspects and embodiments of the present disclosure;

FIG. 69 depicts an illustration of a delivery device in a delivery state in accordance with various aspects and embodiments of the present disclosure;

FIG. 70A depicts a view of an example main body which may be included in a delivery device in accordance with various aspects and embodiments of the present disclosure;

FIG. 70B depicts a side view of an example main body which may be included in a delivery device in accordance with various aspects and embodiments of the present disclosure;

FIG. 71 depicts a detailed view of a portion of an exemplary main body in accordance with various aspects and embodiments of the present disclosure;

FIG. 72A depicts a view of an example main body which may be included in a delivery device in accordance with various aspects and embodiments of the present disclosure;

FIG. 72B depicts a side view of an example main body which may be included in a delivery device in accordance with various aspects and embodiments of the present disclosure;

FIG. 73A depicts a top front right perspective view of an example portion of an example delivery device in accordance with various aspects and embodiments of the present disclosure;

FIG. 73B depicts a bottom plan view of an example portion of an example delivery device in accordance with various aspects and embodiments of the present disclosure;

FIG. 73C depicts a top plan view of an example portion of an example delivery device in accordance with various aspects and embodiments of the present disclosure;

FIG. 73D depicts a front side view of an example portion of an example delivery device in accordance with various aspects and embodiments of the present disclosure;

FIG. 73E depicts a right side view of an example portion of an example delivery device in accordance with various aspects and embodiments of the present disclosure;

FIG. 73F depicts a back side view of an example portion of an example delivery device in accordance with various aspects and embodiments of the present disclosure;

FIG. 73G depicts a left side view of an example portion of an example delivery device in accordance with various aspects and embodiments of the present disclosure;

FIG. 74A depicts a top front right perspective view of an example portion of an example delivery device in accordance with various aspects and embodiments of the present disclosure;

FIG. 74B depicts a top plan view of an example portion of an example delivery device in accordance with various aspects and embodiments of the present disclosure;

FIG. 74C depicts a bottom plan view of an example portion of an example delivery device in accordance with various aspects and embodiments of the present disclosure;

FIG. 74D depicts a front side view of an example portion of an example delivery device in accordance with various aspects and embodiments of the present disclosure;

FIG. 74E depicts a right side view of an example portion of an example delivery device in accordance with various aspects and embodiments of the present disclosure;

FIG. 74F depicts a back side view of an example portion of an example delivery device in accordance with various aspects and embodiments of the present disclosure;

FIG. 74G depicts a left side view of an example portion of an example delivery device in accordance with various aspects and embodiments of the present disclosure;

FIG. 75A depicts a top front right perspective view of an example portion of an example delivery device in accordance with various aspects and embodiments of the present disclosure;

FIG. 75B depicts a top plan view of an example portion of an example delivery device in accordance with various aspects and embodiments of the present disclosure;

FIG. 75C depicts a bottom plan view of an example portion of an example delivery device in accordance with various aspects and embodiments of the present disclosure;

FIG. 75D depicts a front side view of an example portion of an example delivery device in accordance with various aspects and embodiments of the present disclosure;

FIG. 75E depicts a right side view of an example portion of an example delivery device in accordance with various aspects and embodiments of the present disclosure;

FIG. 75F depicts a back side view of an example portion of an example delivery device in accordance with various aspects and embodiments of the present disclosure;

FIG. 75G depicts a left side view of an example portion of an example delivery device in accordance with various aspects and embodiments of the present disclosure;

FIG. 76A depicts a top plan view of an example reservoir in accordance with various aspects and embodiments of the present disclosure;

FIG. 76B depicts a bottom plan view of an example reservoir in accordance with various aspects and embodiments of the present disclosure;

FIG. 77 depicts a perspective view of an example septum in accordance with various aspects and embodiments of the present disclosure;

FIG. 78 depicts a cross-sectional view of an example reservoir including a septum in accordance with various aspects and embodiments of the present disclosure;

FIG. 79 depicts a perspective view of an example delivery device including a septum in accordance with various aspects and embodiments of the present disclosure;

FIG. 80A depicts a perspective cross-sectional view of an example main body including retention tabs in accordance with various aspects and embodiments of the present disclosure;

FIG. 80B depicts a detailed view of the indicated region of FIG. 80A in accordance with various aspects and embodiments of the present disclosure;

FIG. 81A depicts a bottom plan view of an example delivery device with an example adhesive member in accordance with various aspects and embodiments of the present disclosure;

FIG. 81B depicts a bottom plan view of another example delivery device with another example adhesive member in accordance with various aspects and embodiments of the present disclosure;

FIG. 81C depicts a bottom plan view of another example delivery device with another example adhesive member in accordance with various aspects and embodiments of the present disclosure;

FIG. 82A is a perspective view diagram of an example delivery device in accordance with various aspects and embodiments of the present disclosure;

FIG. 82B is a perspective view diagram of an example delivery device in accordance with various aspects and embodiments of the present disclosure;

FIG. 83A is a side view diagram of an example delivery device in a first state in accordance with various aspects and embodiments of the present disclosure;

FIG. 83B is a side view diagram of an example delivery device in a second state in accordance with various aspects and embodiments of the present disclosure;

FIG. 83C is a side view diagram of an example delivery device in a third state in accordance with various aspects and embodiments of the present disclosure;

FIG. 84A is a perspective view diagram of an example delivery device in accordance with various aspects and embodiments of the present disclosure;

FIG. 84B is a cutaway view diagram of an example flexure of a delivery device in accordance with various aspects and embodiments of the present disclosure;

FIG. 84C is a perspective view diagram of an example delivery device in accordance with various aspects and embodiments of the present disclosure;

FIG. 85A is a perspective view diagram of an example delivery device in accordance with various aspects and embodiments of the present disclosure;

FIG. 85B is a perspective view diagram of an example delivery device in accordance with various aspects and embodiments of the present disclosure;

FIG. 86 is a perspective view diagram of an example delivery device in accordance with various aspects and embodiments of the present disclosure;

FIG. 87 is a plan view diagram of an example delivery device in accordance with various aspects and embodiments of the present disclosure;

FIG. 88A is a side view diagram of an example delivery device in a first state in accordance with various aspects and embodiments of the present disclosure;

FIG. 88B is a side view diagram of an example delivery device in a second state in accordance with various aspects and embodiments of the present disclosure;

FIG. 88C is a side view diagram of an example delivery device in a third state in accordance with various aspects and embodiments of the present disclosure;

FIG. 89 is a cross section view diagram of an example delivery device in accordance with various aspects and embodiments of the present disclosure;

FIG. 90A is a block diagram of an example delivery device in a storage state in accordance with various aspects and embodiments of the present disclosure;

FIG. 90B is a block diagram of an example delivery device in a delivery state in accordance with various aspects and embodiments of the present disclosure;

FIG. 91 is a perspective view diagram of an example delivery device in accordance with various aspects and embodiments of the present disclosure;

FIG. 92 is a perspective view diagram of an example delivery device in accordance with various aspects and embodiments of the present disclosure;

FIG. 93 is an exploded view diagram of an example delivery device in accordance with various aspects and embodiments of the present disclosure;

FIG. 94 is an exploded view diagram of an example delivery device in accordance with various aspects and embodiments of the present disclosure;

FIG. 95A is a side view diagram of an example delivery device in a first state in accordance with various aspects and embodiments of the present disclosure;

FIG. 95B is an enlarged view of the indicated region of the delivery device of FIG. 95A in accordance with various aspects and embodiments of the present disclosure;

FIG. 96A is a side view diagram of an example delivery device in a second state in accordance with various aspects and embodiments of the present disclosure;

FIG. 96B is an enlarged view of the indicated region of the delivery device of FIG. 96A in accordance with various aspects and embodiments of the present disclosure;

FIG. 97 is a cross section view diagram of an example delivery device in a first state in accordance with various aspects and embodiments of the present disclosure;

FIG. 98 is a cross section view diagram of an example delivery device in a second state in accordance with various aspects and embodiments of the present disclosure;

FIG. 99 depicts an exploded view of an example delivery device in accordance with various aspects and embodiments of the present disclosure;

FIG. 100 depicts an exploded view of an example delivery device in accordance with various aspects and embodiments of the present disclosure;

FIG. 101 is a perspective view diagram of an example flexure which may be included as or as part of an actuation assembly of an exemplary delivery device in accordance with various aspects and embodiments of the present disclosure;

FIG. 102 is a side view diagram of an example flexure which may be included as or as part of an actuation assembly of an exemplary delivery device in accordance with various aspects and embodiments of the present disclosure;

FIG. 103 is a plan view diagram of an example flexure which may be included as or as part of an actuation assembly of an exemplary delivery device in accordance with various aspects and embodiments of the present disclosure;

FIG. 104 depicts a perspective view of an example package for a delivery device in accordance with various aspects and embodiments of the present disclosure;

FIG. 105 depicts a view of an example main body of a delivery device separated for an example reservoir assembly which may be included in a delivery device in accordance with various aspects and embodiments of the present disclosure;

FIG. 106 depicts a view of an example delivery device being removed from skin of a patient revealing a mark created on the skin by the delivery device in accordance with various aspects and embodiments of the present disclosure; and

FIG. 107 depicts an example thermal image of an injection site in which a bleb from an injection into the skin is visible in accordance with various aspects and embodiments of the present disclosure.

### DETAILED DESCRIPTION

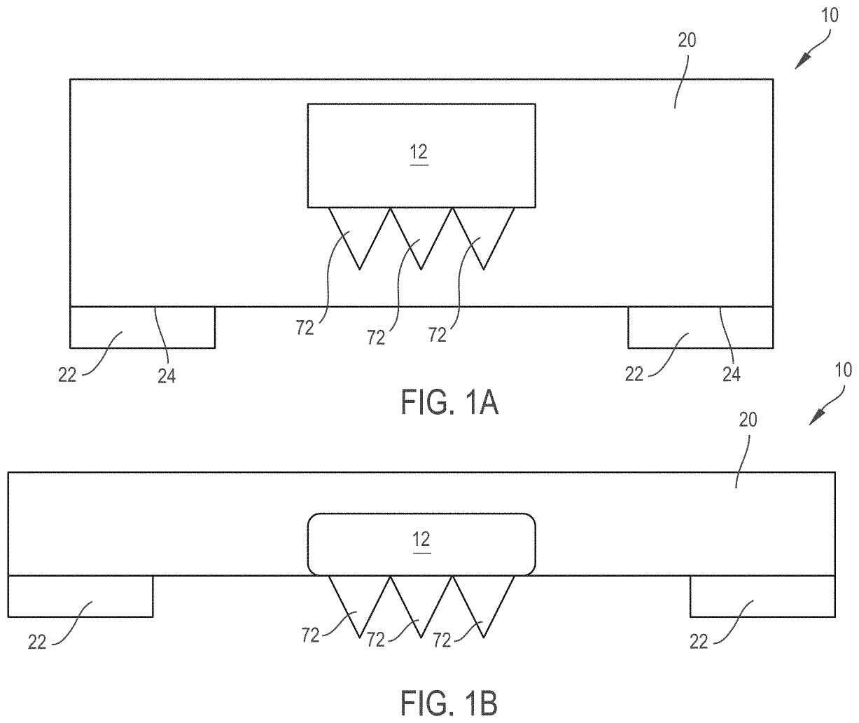

FIG. 1A and FIG. 1B depict an embodiment of an exemplary delivery device 10. The example delivery device 10 may be a low profile delivery device 10 which may be applied over the skin of a patient. The example delivery device 10 may be sized for handheld use and may be easily applied to a wide variety of injection sites over a patient's body. Additionally, the example delivery device 10 may be designed for use by a patient or relatively untrained or minimally trained individual. Thus a medical caregiver may not be necessary for use of the delivery device 10.

Such delivery devices 10 may be used to dispense a medical agent from a reservoir 12 included as part of the delivery device 10 into a target delivery destination of a patient via one or more delivery sharp 72. The reservoir 12 may be at least partly flexible and may have a variable volume which may deplete as fluid is dispensed from the reservoir 12. As the reservoir 12 depletes, the reservoir 12 may at least partially collapse. In the example embodiment, a plurality of delivery sharps 72 are included in the delivery device 10, though other embodiments may only include a single delivery sharp 72. The exemplary plurality of delivery sharps 72 may be arranged in a one or two dimensional array and may extend from the reservoir 12. Where multiple delivery sharps 72 are included, the delivery sharps 72 may be arranged in one or more rows and/or columns. Though three delivery sharps 72 arranged in a single row are depicted in FIG. 1A, the number and arrangement of delivery sharps 72 may differ in alternative embodiments. Any suitable number of rows and/or columns may be included in various examples. In various embodiments there may, for example, be a single row array of delivery sharps 72 including up to five delivery sharps 72. Preferably, the delivery sharps 72 may be arranged so as to prevent a bed of nails type scenario in which penetration of the skin via the delivery sharps 72 may be inhibited or inconsistent across users or delivery devices 10. This may occur when too many delivery sharps 72 are arranged in close proximity to one another. Thus, the array may be referred to as a spaced array of delivery sharps 72.

The delivery sharps 72 may be selected based on the desired target delivery destination in a patient. In certain embodiments, the target delivery destination may be a transcutaneous location. For example, the target delivery destination may be a subcutaneous delivery destination or an intramuscular delivery destination. Alternatively, the target delivery destination may be a shallow delivery destination between the stratum corneum of a patient and the subcutaneous tissue of the patient. Such shallow destinations may be referred to herein as intradermal delivery destinations. Shallow delivery destinations may include an epidermal or dermal target location or may, for example, target a junctional area between the epidermis and dermis or dermis and subcutis. In the example embodiment, the delivery sharps 72 are depicted as microneedles. Such delivery sharps 72 may be present in delivery devices 10 with shallow (e.g. above subcutaneous tissue) target delivery destinations. In alternative embodiments where, for instance, the target delivery destination is a subcutaneous or intramuscular location, conventional delivery sharps (e.g. 30-gauge needle) may be utilized.

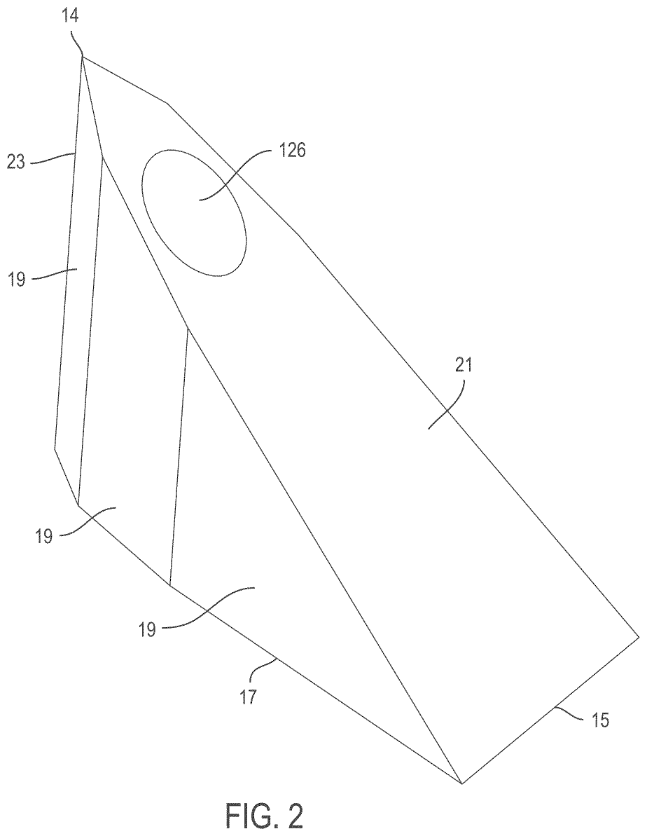

Referring now also to FIG. 2, where microneedles are used, the microneedles described herein may, in certain embodiments, be MEMS produced, polyhedral (e.g. pyramidal), silicon crystal microneedles. These microneedles may be no greater than 1 mm in height, e.g. 0.6 mm or 0.8 mm (though longer or shorter microneedles may also be used). At least some edges of the microneedles may be rounded or filleted, though such microneedles may still be referred to herein as polyhedral. In some examples and as shown in FIG. 2, the microneedles described herein may be generally in the shape of a heptagonal prism (though pentagonal, nonagonal, and other polygonal prisms may also be used as the base shape) which has been diagonally sected to form a heptagonal ramp or pointed wedge. In such embodiments, the heptagonal prism may be sected by a plane extending from a vertex 14 of the top face of the prism through the most distal side 15 of the base 17. At least two sides of the base of the microneedle may be parallel. The side walls 19 may extend substantially perpendicularly from the base 17. The microneedle may be substantially symmetric about a line of symmetry extending from the vertex 14 to a point above the center of the most distal side 15. In other embodiments, the microneedles may be conically shaped. Any other suitable shape may be used. In the example, the vertex 14 is shown as a point which forms a tip of the microneedle. In other embodiments, this portion of a microneedle may be rounded (though may still be referred to herein as a vertex 14 and such microneedles may still be referred to as pointed). In such embodiments, the back facing edge 23 may be a round face or the back facing edge 23 and the adjacent side walls 19 may be replaced by a rounded face.

The points or tips of microneedles described herein may be solid and the flow lumens 126 through the microneedles may be offset from the points or tips (in FIG. 2 the vertex 14 forms the tip) of the microneedles. Hollow tipped microneedles in which the flow lumen 126 extends to the tip of the microneedle may also be utilized. In some embodiments, the microneedles may be NanoPass hollow microneedles available from NanoPass Technologies Ltd. of 3 Golda Meir, Nes Ziona, Israel. It should be noted that microneedles (or the substrate on which they are disposed) described herein as constructed of silicon may have a surface layer of silicon dioxide (which may, for example, form with exposure to air) while still being considered constructed of silicon.

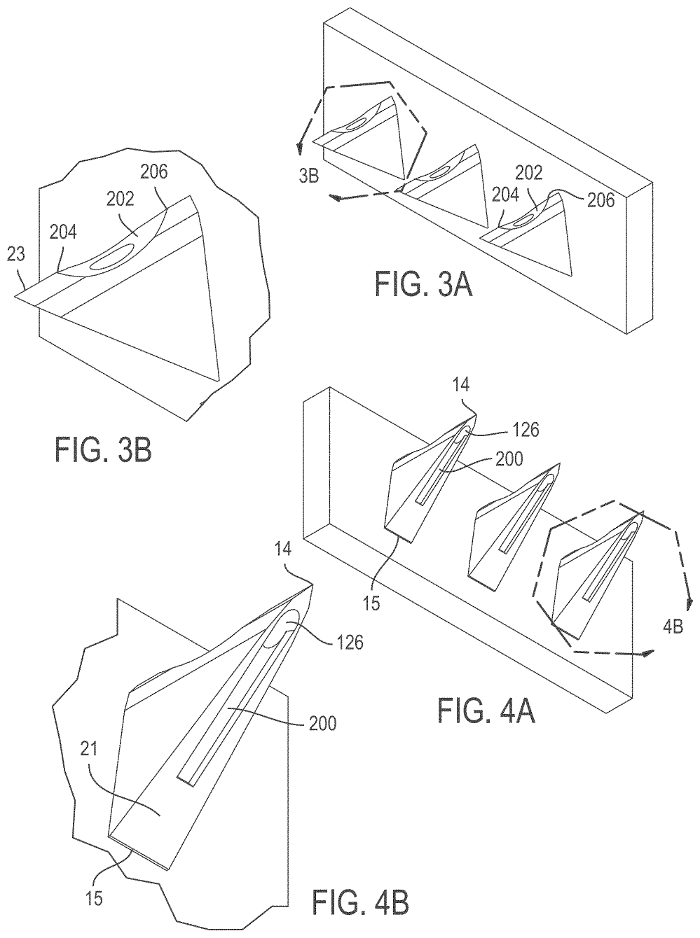

With reference to FIGS. 3A-4B, in some embodiments microneedles may be constructed to include certain features that may help to reduce the pressure required to inject fluid, such as a medical agent, into the skin of a patient. In some examples, features common certain to insect stingers or biological venom administration structures may be incorporated. These features may include various recesses or depressions which are formed as part of each microneedle or at least one microneedle of a delivery device 10. These recesses or depressions may fluidly communicate with the flow lumen 126 of the respective microneedle. In some embodiments, different microneedles of a delivery device 10 may include different recesses or some microneedles may include a plurality of recesses which could be of different varieties (though need not be).

For example, as shown in FIGS. 4A-4B, a microneedle may include a channel or trough 200 on an exterior sloped face 21 leading from the flow lumen 126 toward the distal side 15. The channel 200 may allow medical agent to flow through it along the outer side of the microneedle to find a path of least resistance, or weakest link, into the skin. In the embodiments shown, medical agent may be routed by the channel 200 to flow along the outer side of the microneedle to a weak region in the skin in the event the outlet of the flow lumen 126 has been inserted to a greater depth than the depth of the weak region. The lamina lucida junction, an intradermal delivery destination, is a weak link in the skin structure, and is difficult to consistently inject directly into due to its relative thinness (it is typically on the order of 40 nm thick). A microneedle including a channel 200 may, for example, allow flow of medical agent to the lamina lucida junction when the lamina lucida junction has been passed by the outlet of the flow lumen 126. The channel 200 may facilitate distribution of the medical agent through a larger area of entry or injection. In some examples, incorporating a channel 200 into a microneedle may reduce the pressure required to inject a medical agent into the skin considerably. In certain examples pressure may be reduced by 600% or more (e.g. from 120 pounds per square inch (psi) to from 18 to 20 psi in certain examples).

An appropriate silicon etching technique (or mold in embodiments using polymeric microneedles) may be used to create steeper side walls of the channel 200. This may help inhibit the skin from bending into and occluding the channel 200. Etching techniques that could be used include, by way of non-limiting example, chemical etching techniques (e.g., acid). Suitable etching techniques may include ion based etching techniques (e.g. reactive ion etching). The etching process could be a wet etching process or a dry etching process. In some non-limiting embodiments, the channel 200 may be within a range of 50-60 microns wide from side to side. In some non-limiting embodiments, the flow lumen 126 may have a diameter of 50-60 microns. The channel 200 may have a width equal to the diameter or widest portion of the flow lumen 126 or the channel 200 may have a width which is less than or greater than the width of the flow lumen 126. In certain examples, the width of the channel 200 may be about 5-10 percent of the height of the microneedle.

To avoid leakage of the fluid from the channel 200, it may be desirable to ensure that the channel 200 terminates at least a certain distance beneath the surface of the skin yet also reaches the targeted skin layer (e.g., the lamina lucida junction) when the microneedle is inserted into the skin. In some embodiments the channel 200 extends from the flow lumen 126 to within at most 50 microns (e.g. 50-200 microns) of the base 17 of the microneedle. In some embodiments, the end of the channel 200 most proximal the base 17 of the microneedle may be at least below the stratum corneum (and perhaps one or more of the stratum lucidum, stratum granulosum, stratum spinosum, and stratum basale) when the microneedle is inserted into the skin. In some embodiments, the end of the channel 200 most proximal the base 17 may be disposed below the epidermis (e.g. in the basement membrane) or within the epidermis.

The channel 200 need not be straight or shaped in the manner shown in and described with reference to FIGS. 4A-4B. In some embodiments, the channel 200 may be a more meandering channel 200. A curved channel 200 could, for example, be used provided the dimensions of the microneedle are accommodated. Moreover, there need not be only one channel 200. More than one channel could be used provided structural integrity of the microneedle is accommodated.

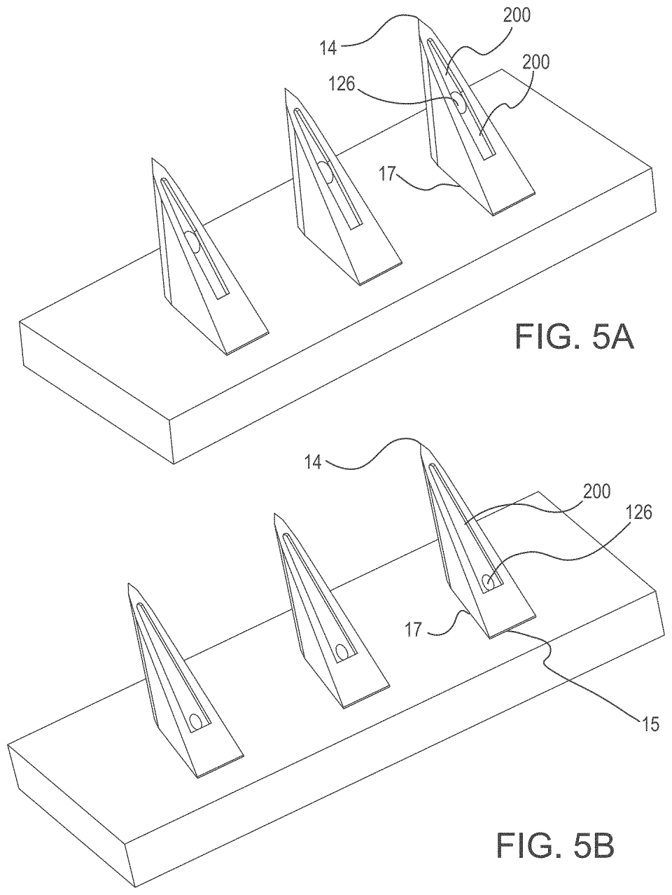

The depth of the channel 200 may be about 25 microns or more (e.g. 25-50 microns) in certain examples. The depth of the channel 200 may be or be less than 5 percent the height of the microneedle. While the depth of the channel 200 may be constant along the length of the channel 200, the depth of the channel 200 need not be constant along the length of the channel 200. Likewise, the width of the channel 200 need not be constant along the length of the channel 200 (see, e.g., FIG. 5B). The width of the channel 200 may be about 20-30 percent of the width of the distal side 15 of the microneedle at the narrowest point in the channel 200. In some embodiments, the width of the channel 200 may increase as distance to the distal side 15 decreases. In some embodiments, at its widest, the channel 200 may have a width which is 50% or more the width of the distal side 15.

Referring now also to FIG. 5A and FIG. 5B, in other examples, the channel 200 may extend from the location of the lumen 126 toward the tip or vertex 14 of the microneedle (see, e.g., FIG. 5B). Moreover, in some examples, the channel 200 may extend both toward the vertex 14 and toward the base 17 from the location of the lumen 126. That is, the channel 200 may include a portion on both sides of the lumen 126 (see, e.g., FIG. 5A). As shown, the lumen 126 may be located substantially centrally in the sloped face 21 of the microneedle. In such embodiments, a channel 200 may extend toward the distal side 15 of the base 17 and a channel 200 may extend toward the tip or vertex 14. In other embodiments, the lumen 126 may be positioned at (or near) an end of the channel 200 most proximal the base 17.

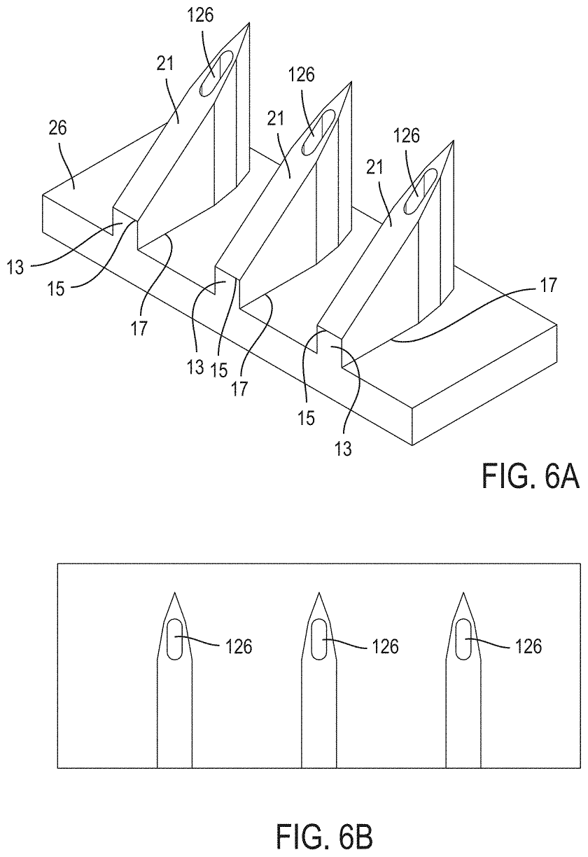

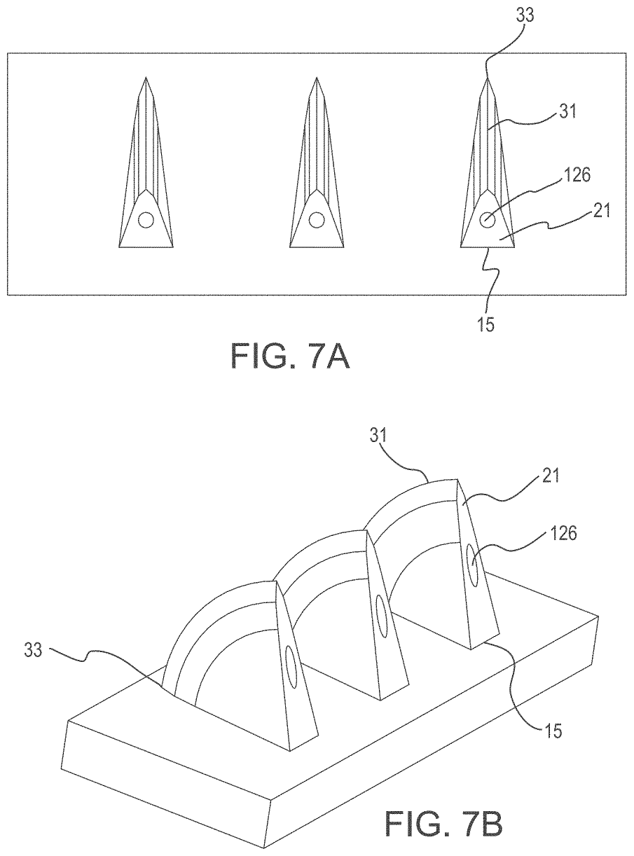

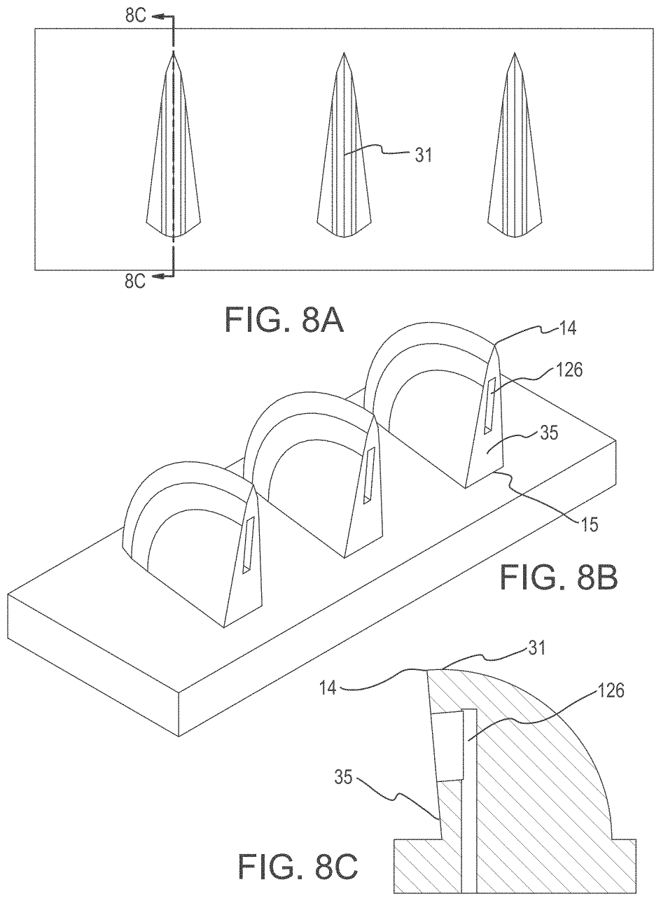

Referring now to FIGS. 6A-6B, views of a sharp bearing body 26 including a number of microneedles are shown. In certain embodiments, a channel 200 may not be included. Instead, a microneedle may include a flow lumen 126 with an elongate cross-section (at least at the outlet, see also FIG. 7B and FIG. 8B). Microneedles with channels 200 and elongate lumens 126 are also possible. When in place within the patient, an elongate lumen 126 may be in fluid communication with, for example, multiple layers of skin. Thus, a thin and/or weak layer of skin may be easier to target when the microneedle is advanced into a patient. Elongate lumens 126 may also help to lower pressure required to inject. Such elongate flow lumens 126 may have any suitable cross-section. In some embodiments, the cross-section may be oval or elliptical. Alternatively, a lumen 126 with an obround cross-section may be used as is shown in FIGS. 6A-6B. Polygonal cross-sectional shapes may also be used, such as though not limited to rectangular, trapezoidal, triangular, etc. In certain examples, the length (in the direction of elongation) of the cross-section of the lumen 126 may be up to 100-200 microns or greater (though could be less in certain examples). Where elongate lumens 126 are included, the end of the lumen 126 most proximal the distal side 15 may be spaced from the distal side 15 by at least a certain distance. The spacing may be such that, the end of the lumen 126 most proximal the distal side 15 may be at least below the stratum corneum (and perhaps one or more of the stratum lucidum, stratum granulosum, stratum spinosum, and stratum basale) when the microneedle is inserted into the skin. In some embodiments, it may be disposed below the epidermis (e.g. in the basement membrane) or within the epidermis.

Still referring to FIGS. 6A-6B in certain embodiments, the sloped face 21 of a microneedle may not extend to the base 17 of a microneedle. There may, for example, be a vertical face 13 extending from the base 17 to the distal side 15 of a microneedle. Where a vertical face 13 is included, the vertical face 13 may be aligned with a side (e.g. distal side 15) of a sharp bearing body 26 and may form an extension thereof. Including such vertical faces 13 may aid in reducing the size of a sharp bearing body 26 and may aid in ensuring consistent fluid delivery into a target destination for certain microneedles. Though shown in relation to FIGS. 6A-6B, any of the microneedles shown herein may be arranged with vertical faces 13.

Additionally or in the alternative, a microneedle may include a depression 202. The depression 202 may include first and second opposing vertices 204, 206. In some embodiments, the depression 202 may be (though need not necessarily be) a rounded depression or a concave depression, as shown in FIGS. 3A-3B. The depression 202 may have a maximum depth which places the depression 202 into fluid communication with the flow lumen 126 of the microneedle. The depression 202 may thus form a side port for the microneedle through which fluid may be delivered to the patient. The side port may be the only outlet of the microneedle or may be in addition to an outlet of the lumen in the face 21 of the microneedle. When the microneedle is inserted into the skin surface, fluid contained in a delivery device 10 may be delivered to the patient, at least in part, by being pumped into the depression 202. The depression 202 may be formed, for example by cutting away material during manufacture of the microneedle or the depression 202 may be formed during a molding operation. Cutting away material may be accomplished by any known suitable process such as, for instance, etching (e.g. wet etching). In some embodiments, the depression 202 may be recessed in at least one side wall 19 or edge (e.g. where two side walls 19 join) of the microneedle. In the example shown in FIGS. 3A-3B, the depression 202 is formed in a substantially vertical back facing edge 23 of the microneedles which extends from the base 17 to the vertex 14. This may establish or increase a vertical void volume created by the microneedle as the skin is penetrated by the microneedle. That is, such a depression 202 may establish an open space in a patient into which fluid may be easily delivered from the microneedle. Positioning the depression 202 in the back facing edge 23 may provide a path of low resistance for a fluid to enter skin that the microneedle has penetrated. In embodiments wherein the microneedle includes at least one substantially vertical wall, the depression 202 may be recessed into a substantially vertical wall. In the example embodiment, the maximum depth of the depression 202 may be about 130% to 110% of the distance from the back facing edge 23 to the flow lumen 126.

In certain examples, and referring now to FIG. 7A and FIG. 7B, a microneedle may include a sloped face 21 to which a lumen 126 extending through the microneedle extends. A microneedle may also include a rounded blade edge 31. In the example, the rounded blade edge 31 extends from a point 33 opposite the distal side 15 and extends in an arcuate path to the vertex or tip 14 of the microneedle. In the example, the rounded blade edge 31 includes a double bevel, though other bevel types may be used. The rounded blade edge 31 may arc at a constant radius or a variable radius. The rounded blade edge 31 may have an arc measure of less than 90° or, in certain examples, greater than 90° (see, e.g., FIG. 8A-8C). The rounded blade edge 31 may aid in introduction of a microneedle into skin when the microneedle is inserted at certain angles or over a variety of different angles.

In yet another embodiment, and referring now to FIGS. 8A-8C, a microneedle may include a rounded blade edge 31 and a lumen outlet face 35. The lumen 126 may extend through the microneedle to the lumen outlet face 35 and may not be formed in a straight line through the microneedle. The lumen outlet face 35 may be angled from the vertex 14 to the distal side 15 so as to form an undercut. The distal edge 15 may be disposed such that a plane perpendicular to the base 17 passing through the distal edge 15 may also pass through the rounded or arcuate blade edge 31. Additionally, the outlet of the flow lumen 126 in the lumen outlet face 35 may be disposed such that a plane or all planes perpendicular to the base 17 and passing through the outlet of the flow lumen 126 may also pass through the blade edge 31. This need not be true in all embodiments (see, e.g., FIGS. 7A-7B). As a microneedle of the variety shown in FIGS. 8A-8C is inserted, a vertical void space may be created due to the undercut. This may provide a low resistance pathway for fluid injection. Additionally, the undercut may help to mitigate potential for the lumen 126 to become obstructed by skin as the microneedle is inserted into a patient or as the delivery occurs.

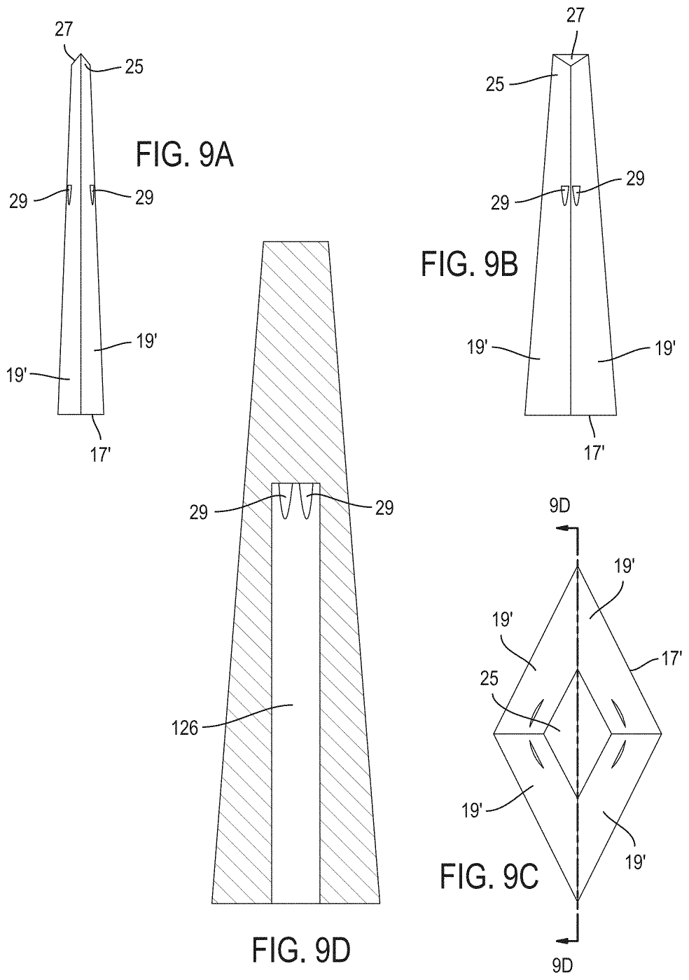

In still other embodiments and referring now to FIGS. 9A-9D, the delivery sharp(s) 72 may be or include a microneedle which has a shape with a high aspect ratio. In some embodiments, microneedles may be obelisk shaped. Such microneedles may be included in an array such as any array described herein. Where obelisk type microneedles are used, the microneedles may include a base 17′. The base 17′ may be any desired round or polygonal shape. For purposes of example, FIGS. 9A-9D depict a base 17′ which is a quadrilateral or rhombus. The example microneedle includes a set of sidewalls 19′ which extend from the base 17′ to an end region 25 of the microneedle. The sidewalls 19′ may be disposed at an angle which is not perpendicular to the base 17′. Thus the microneedle may taper so as to have a smaller cross-sectional area as distance from the base 17′ increases. A portion of the microneedle most distal to the base 17′ may include a beveled tip 27. Such a tip 27 may facilitate puncture of the skin and may aid in increasing the robustness of the end region 25. Any suitable bevel such as a single or double bevel may be used.

In embodiments of microneedles which are obelisk shaped, the microneedles may include at least one side port 29 which may serve as an outlet for that microneedle. Such side port(s) 29 may be difficult to block off with tissue which that may become compressed during insertion of the microneedle into a patient. In the example embodiment, a lumen 126 may extend through the base 17′ of the microneedle and have a terminal end which is more proximal the end region 15 than the base 17′. The lumen 126 may be of relatively constant cross-section. The taper of the sidewalls 19′ may be such that the terminal end of the lumen 126 is wider than portions of the cross-section of the corresponding region of the microneedle. Thus, the lumen 126 may form openings in the sidewalls 19′ which may serve as the side ports 29. In various examples, the lumen 126 may be centrally disposed yielding symmetrical side ports 29. In alternative embodiments, the lumen 126 need not be centrally disposed and the side ports 29 may not be symmetrical.

In various embodiments where silicon is not used to form the microneedles, microneedles described herein may be constructed of glass (e.g. silica glass, borosilicate glass), ceramic (e.g. alumina, calcium sulfate dehydrate, calcium phosphate dehydrate, organically modified ceramics such as Ormocer), polymer (e.g. polymethyl methacrylate or PMMA, polylactic acid or PLA, polylactic-co-glycolic acid or PLGA, polyglycolic acid or PGA, polycarbonate, cyclic-olefin copolymer or COC, polyvinylpyrrolidone or PVP, polyvinyl alcohol PVA, polystyrene, polymethyl vinyl ether-co-maleic anhydride), carbohydrate, or metal (e.g. stainless steel, titanium, palladium, nickel, alloys such as palladium cobalt alloys, etc.). Any suitable microneedle constructions including dissolvable microneedles may be used. Microneedles and features thereof may be manufactured in one or more of, though are not limited to, a molding process, etching process, ablative process (e.g. laser ablation), or a material additive process (e.g. 3D printed). In various embodiments, it may be desirable that microneedles be constructed of a biocompatible, non-ductile, high Young's modulus material with an indentation hardness sufficient to allow penetration into skin without breakage.

Referring again primarily to FIGS. 1A and 1B, delivery devices 10 described herein may deliver any of a variety of medications or other medical agents to a patient. In certain embodiments, the reservoir 12 of the delivery device 10 may be filled with a vaccine. Such a delivery device 10 may deliver any suitable vaccine, though may be particularly well suited to vaccines for novel pathogens (e.g. SARS-CoV-2) or for pathogens where herd immunity does not exist (e.g. Ebola). Additionally, such delivery devices 10 may be of particular usefulness in outbreaks of pathogens (such as measles for example) in communities which choose to forego typical vaccinations. For example, such delivery devices 10 could be distributed without requiring patients to congregate in hospitals or other shared spaces. This would mitigate concern for pathogen transmission related to vaccination programs and alleviate potential worries that could dissuade people from reporting to receive a vaccination. Instead, delivery devices 10 could be picked up and used by patients without breach of social distancing, gathering size recommendations, or other safety guidelines. Alternatively, such delivery devices 10 could be distributed directly to patients without requiring a patient to leave their domicile or requiring distribution personnel to interact with individuals who decline to utilize recommended PPE. Delivery devices 10 could be filled with a vaccine for a novel pathogen or could perhaps be filled with vaccines typical of a normal vaccination schedule. In the latter case, such a delivery device 10 could help to ensure that disruption of vaccination for known pathogens does not occur during a novel pathogen pandemic.

Any suitable vaccine may be delivered via such a delivery device 10. For example, the vaccine may be but is not limited to, attenuated live vaccines, inactivated virus vaccines, acellular vaccines, cellular vaccines, toxoid vaccines, heterotypic or Jennerian vaccines, monovalent vaccines, polyvalent vaccines, nucleic acid vaccines (e.g. DNA, plasmid vaccine, mRNA), virus like particle vaccines, recombinant vector vaccines (e.g. replicating, non-replicating), dendritic cell vaccines, T-cell receptor peptide vaccines, chimeric vaccines, subunit vaccines, nanoparticle vaccines, recombinant protein vaccines, polysaccharide vaccines, and conjugate vaccines. It should be noted that these are not necessarily mutually exclusive. For instance, a vaccine could be a recombinant protein nanoparticle vaccine or some other combination of the above. Vaccine may also refer to a combination vaccine (e.g. DTaP, MMR, MMRV, etc.) or a vaccination agent which targets a single pathogen or multiple strains of a single pathogen. Example vaccines may include, but are not limited to vaccines for various coronaviruses such as SARS-COV, SARS-COV-2, MERS-COV, HCoV-NL63, HCoV-229E, HCoV-OC43 and HKU1 (and any variants or sub-variants thereof). Delivery devices 10 described herein are also not limited for use with humans. Such delivery devices 10 may be used for livestock, pets, services animals, or in other veterinary applications. In such cases, these delivery devices 10 may be filled with a vaccine for at least one non-human pathogen. Delivery devices 10 described herein may also be useful for research applications.

Where a delivery device 10 is filled with a vaccine, it may be desirable that the target delivery destination be a shallow delivery destination. This may be particularly desirable where the amount of available vaccine is limited. For example, such a delivery device 10 may be well suited for use with new vaccines having high demand. Vaccines for novel pathogens (e.g. SARS-CoV-2 or other coronaviruses) may, for instance, be well suited for use with delivery devices 10 described herein.

Evidence suggests that shallow delivery of vaccines may provoke protective immune response with smaller amounts of vaccine antigen. As a result, dose sparing may be practiced allowing the same quantity of vaccine to be effective for immunizing a greater number of people. Alternatively or additionally, injection sparing may be possible. Shallow administration with a delivery device 10 such as those shown herein may allow for a single injection protocol where other routes of administration may require multiple injections over some period of time. One or more adjuvants may be included in some vaccine formulations to further aid in facilitating dose or injection sparing, though less reliance on adjuvants could also be possible with when a vaccine is administered intradermally.

Particularly for new vaccines generated to combat an ongoing pandemic (e.g. a vaccine for SARS-CoV-2), the prospect of rapidly generating billions of doses would almost certainly exceed current vaccine production capabilities. Due to the injection and dose sparing potential of delivery devices 10 described herein, such delivery devices 10 may facilitate vaccination of large numbers of people even when a critically needed vaccine is in short supply. Additionally, as a consequence of potential dose and injection sparing, delivery devices 10 such as those shown and described herein may allow injections to be more cost effective. Moreover, due to the small volume of vaccine needed, delivery devices 10 may be made relatively small. This may simplify shipping and help to facilitate rapid distribution of vaccine to a population. This may be particularly attractive for vaccines which require cold chain distribution as packing volume may be of heightened importance.

Additionally, some studies have suggested that shallow administration may be particularly helpful in certain patient populations. For example, elderly populations may receive superior protection from vaccinations received intradermally than via other routes. That said, the Mantoux technique, which is typically used for intradermal administration, can pose reliability concerns and can be difficult to perform, especially without training. Per the World Health Organization, a large factor which has limited the use of intradermal vaccination has been the lack of a delivery platform.

Delivery devices 10, such as those shown and described herein, may provide an attractive delivery platform for intradermal vaccination. Consequentially, delivery devices 10 described and shown herein may help to give better protection to vulnerable populations and may help in meeting the large demand for vaccines against, for example, novel pathogens by leveraging dose/injection sparing which may be possible with intradermal vaccination. Moreover, intradermal delivery devices 10 described herein may be painless or nearly pain free which may make the delivery devices 10 described herein user preferable over other types of injections. That said, and as mentioned above, delivery devices 10 described herein are not limited to delivery via the intradermal route. Delivery devices 10 may, for instance, be configured as transdermal (e.g. subcutaneous or intramuscular) delivery devices 10.

The example delivery devices 10 shown herein additionally are not limited to vaccine delivery devices. Such a delivery device 10 may fill a number of niches in the medical field. Other agents, for example, diagnostic or testing agents may be supplied via certain example delivery devices 10. For instance, allergens or potential allergens may be administered via the delivery device 10. Tuberculosis testing agents may be delivered via the delivery device 10. Such delivery devices 10 may also be used to deliver medication for endocrine disorders. For instance, insulin may be delivered with some exemplary delivery devices 10.

Delivery devices 10 described and shown herein may also be well suited to deliver drugs for overdose intervention such as opioid antagonists (e.g. Naloxone). Delivery devices 10 described herein may be easily used at the site of an overdose by a non-medically trained bystander. Alternatively, delivery devices 10 may be used by emergency medical services (EMS) personnel responding to an overdose. Such delivery devices 10 may provide rapid access and delivery to, for instance, a shallow destination in the overdose victim. This may allow agent to be supplied to an overdose victim in a rapid manner. Additionally, it may obviate the need to establish an intravenous access which can be particularly difficult for users of intravenous drugs.

Still referring to FIGS. 1A-1B, the delivery device 10 may include a main body 20. The main body 20 may be a deformable body which may transition from a storage state (see FIG. 1A) to a delivery state (see FIG. 1B). In certain examples, this transition may be reversible, though in other embodiments the transition may result in a permanent change in the main body 20 and/or another part of the delivery device 10. For example, once transitioned to the delivery state, the main body 20 may plastically deform such that it is permanently distorted and may not be returned to the storage state. In other examples, a frangible included in the delivery device 10 may be broken upon transition of the main body 20 to the delivery state. Alternatively or additionally, a latch, lock, or other coupling may be engaged to hold the main body 20 in the delivery state or prevent the main body 20 from returning to the storage state. Destruction of a portion of the main body 20 or a portion of the delivery device 10 engaged to the main body 20 may be required to disengage such a coupling and this destruction may render the delivery device 10 inoperative. Where a permanent change is engendered upon transition to the delivery state, this permanent change may inhibit reuse as well as provide a user perceptible (e.g. visual) indication that the delivery device 10 has been used. An indication that the transition has occurred may also be generated by the delivery device 10. For instance, an audible or tactile indication may be generated upon engagement of a latch or breaking of a frangible.

In various examples, transition of the delivery device 10 from the storage state to the delivery state may be accomplished via bending, pivoting, or deformation of one or more regions of the main body 20. In certain examples, the main body 20 may include one or more hinges (e.g. living hinge to aid in lowering part count) at which the main body 20 may bend. In other embodiments, the main body 20 may be or include a bi-stable element which may have a first stable state which corresponds to the storage state and a second stable state which corresponds to the delivery state. The main body 20 may for example substantially or partially invert (e.g. convex to concave) in shape or have one or more invertible regions which at least partially invert when the delivery device 10 is transitioned from the storage state to the delivery state. In some embodiments, the main body 20 may include one or more regions which may invert while also including one or more regions which distort and at least partially restore as a result of the delivery device being transitioned to a delivery state.

The transition may be affected via application of force throughout the entire transition. Alternatively, the transition may only require application of force throughout a portion of the transition. For example, in some embodiments a triggering force may be applied to initiate the transition and the transition may subsequently complete in the absence of any external application of force. For example, after application of the triggering force, the transition may be characterized by a snap-through buckling via which the main body 20 rapidly shifts into the delivery state.

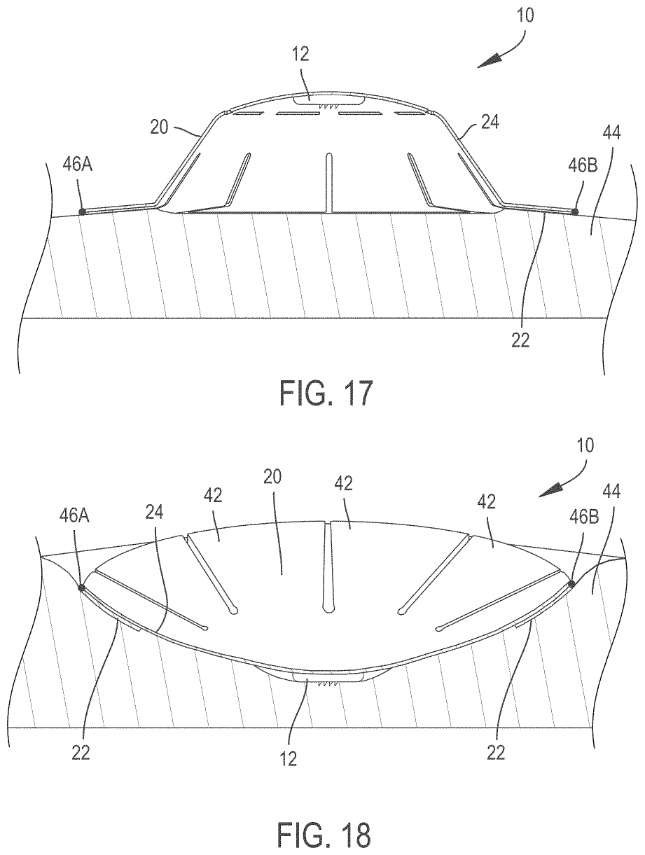

The main body 20 may be at least partially covered with adhesive 22 over a first face 24 of the main body 20. The adhesive 22 may serve to couple the main body 20 to a skin surface at an infusion or injection site on a patient. Thus, the first face 24 may be a skin adjacent face or proximal (proximal and distal defined in relation to a patient) face of the main body 20. The main body 20 may be adhered to the skin when the main body 20 is in the storage state and then may be transitioned to the delivery state. As the transition occurs, at least two adhesive bearing portions of the main body 20 may be displaced with respect to one another so as to stretch or spread a surface anchored to the main body 20 via the adhesive 22. As these portions may be adhered to the skin surface, the skin may be stretched as the adhesive bearing portions are displaced with respect to one another. This may be desirable as the skin may be rendered taught facilitating piercing of the skin by the delivery sharp(s) 72 as the main body 20 transitions to the delivery state. In certain examples, the adhesive bearing portions may be disposed, for example, in opposition to one another. The displacement of the two adhesive bearing portions may increase the distance between or spread apart the two adhesive bearing portions. In other embodiments, the distance between the two adhesive bearing portions may not increase or may even decrease while still causing stretching of the skin surface. This may for example occur if the transition causes a flat patch of skin to be pulled around a curve or contour of the main body 20 (see, e.g., FIGS. 17-18). A displacement of adhesive bearing portions with respect to one another that results stretching of the adhered skin (regardless of any positive or negative change in distance between the adhesive bearing portions) may be referred to as a spreading displacement. Two adhesive bearing portions which have been so displaced may be referred to as being spreadingly displaced.

Transition of the main body 20 to the delivery state may also result in a proximal displacement or lowering of the delivery sharp(s) 72 toward and into the skin. In embodiments where the delivery sharp(s) 72 are coupled to the reservoir 12, the reservoir 12 may also be proximally displaced. In some examples, the reservoir 12 may be compressed between the skin surface and a section of the main body 20 when the main body 20 is transitioned from the storage state to the delivery state. Preferably, the delivery sharp(s) 72 may be inserted into the skin prior to the reservoir 12 being substantially compressed. Compression of the reservoir 12 may serve to drive fluid out of the reservoir 12, through the delivery sharp(s) 72 and into the target delivery destination in the patient. In embodiments described herein, the delivery sharp(s) 72 may be covered prior to use. A fluid communication path from the reservoir 12 out of the delivery sharp(s) 72 may not be available prior to use.

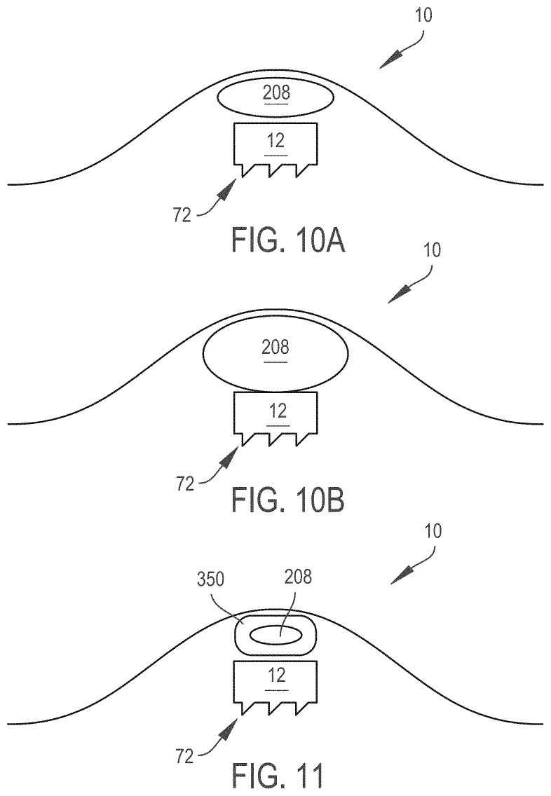

In some embodiments a collapsible pouch, or packet 208, may be positioned in the delivery device 10 above the reservoir 12 as shown in FIG. 10A. The packet 208 may contain a substance that, in a first condition is in a dormant state, and in a second condition transitions to a motive force applying state. In an embodiment the substance may be dormant at a first temperature and apply a motive force, e.g. upon the reservoir 12, at a second temperature. In an example embodiment, the first temperature may be colder than the second temperature. The first temperature may be a cold chain storage temperature for a vaccine. The second temperature may be room temperature or at least below average patient body temperature (e.g. 98.6° F. for humans). In some embodiments, the substance may change in volume when it transitions from a dormant state to a motive force applying state. Alternatively or additionally, the substance may change from one state of matter to another state of matter when it transitions from a dormant state to a motive force applying state. In an example embodiment, the substance may change from a liquid to a gas during the transition from a dormant state to a motive force applying state.

In an embodiment where the medical agent in a reservoir 12 of a delivery device 10 must be stored at very low temperatures, such as, e.g., when the medical agent is a vaccine with such requirements, the packet 208 can contain a liquid. For example, a vaccine may be stored and/or shipped at commercial freezer temperatures, e.g., in the range of −18 degrees C. (or lower, e.g. −70° C. or −20° C. for certain vaccines). The liquid may have a boiling point that is greater than the medical agent (e.g., vaccine) storage temperature but less than room temperature or another suitable temperature set point. Though any suitable liquid may be used, one example of a suitable liquid is butane. Butane has a boiling point of −1° C. Though the example described herein refers to butane, one skilled in the art would appreciate the description is generalizable to any suitable liquid.

The delivery device 10 may be affixed to the skin surface of a user with the reservoir 12 containing cold stored/shipped vaccine and the packet 208 containing liquid butane (or any other suitable substance). If the ambient temperature is room temperature, e.g., about 20 degrees C., the contents will warm up (heat from the patient may aid this). The liquid butane will boil and transition to gas once it reaches its boiling point of −1° C. in the example embodiment. As the liquid boils and transitions to a gaseous state, the pressure in the packet 208 grows, causing it to expand and apply downward pressure on the reservoir 12 from above as shown in FIG. 10B. Butane gas, for instance, has a vapor pressure of 35.4 psi at 25° C. Accordingly, a final pressure on the reservoir 12 may be higher (e.g. around 38 psi) due to heat transfer from the patient to the packet 208. The main body 20 of the delivery device 10 may be sufficiently resilient to not deform under the pressure applied from the packet 208. This may aid in directing pressure against the reservoir 12.

Such an arrangement may also serve to provide visible evidence of whether the delivery device 10 had reached a temperature during storage or shipping that was too high for the medical agent. For example, if a temperature that was beyond the phase change temperature of the packet 208 contents was reached during storage, the delivery device 10 would be emptied due to the pressure applied from above by the packet 208. This may be visibly perceptible to a user. The delivery device 10 would also self-destruct when subjected to certain temperature abuse scenarios. In the event that the delivery device 10 was subjected to a temperature above the phase change temperature of the packet 208 contents, the delivery device 10 would be emptied. As a result, the delivery device 10 would prevent itself from later administering a temperature abused medical agent.

In some embodiments significant downward pressure on the reservoir 12, e.g., greater than 50 psi, may be desired to provide force to collapse the reservoir 12 and force fluid therein into the skin of a user via delivery sharps 72 as described above with reference to FIG. 1A and FIG. 1B. In such embodiments the packet 208 may be incorporated within a squeezable container 350 as shown in FIG. 11. The container 350 may be made of a squeezable plastic or any other suitable material as would be understood by those of skill. The container 350 may be formed by injection molding, thermoforming, or any other technique known to those of skill. In addition to housing the packet 208, a first substance may be stored within the container 350. The packet 208 may hold a second substance. The first and second substances may be components of, for example, and an expanding foam. The first and second substances may be selected such that they expand to create pressure when they come into contact with each other. A chemical reaction (e.g. baking soda and vinegar) which generates gas could for example be used. Upon applying the delivery device 10 to the skin surface, a user may, e.g., pinch, crush, smoosh, or squeeze the container 350. This may cause the packet 208 therein to rupture and thereby allow the first and second substances to interact and cause downward pressure on the reservoir 12 below.

In still other embodiments, the packet 208 could be a vacuum packed bias member (e.g. foam spring). In the vacuum packed state, the bias member may be in a compressed state. User interaction with the packet 208 may cause the packet 208 to break allowing the bias member to restore. As the bias member restores, pressure may be applied to the reservoir 12 to generate pressure for delivery.

In alternative embodiments, a packet 208 of FIG. 10A or FIG. 10B may be filled with contents which do not change phase when removed from cold storage. For example, the packet 208 may be a gas bladder that may serve to prevent pressure from a user's finger applied to the top of the delivery device 10 from being applied directly to the reservoir 12. Such a gas bladder may also help to make applied pressure more uniform across the reservoir 12. An example of a suitable gas which may be used to fill such a packet 208 may be nitrogen. Any other suitable gas may be used.

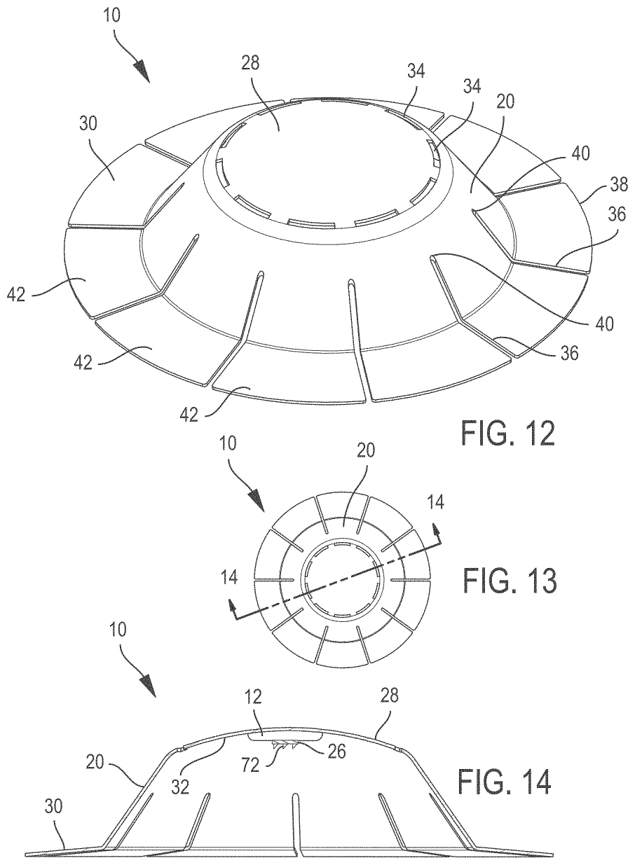

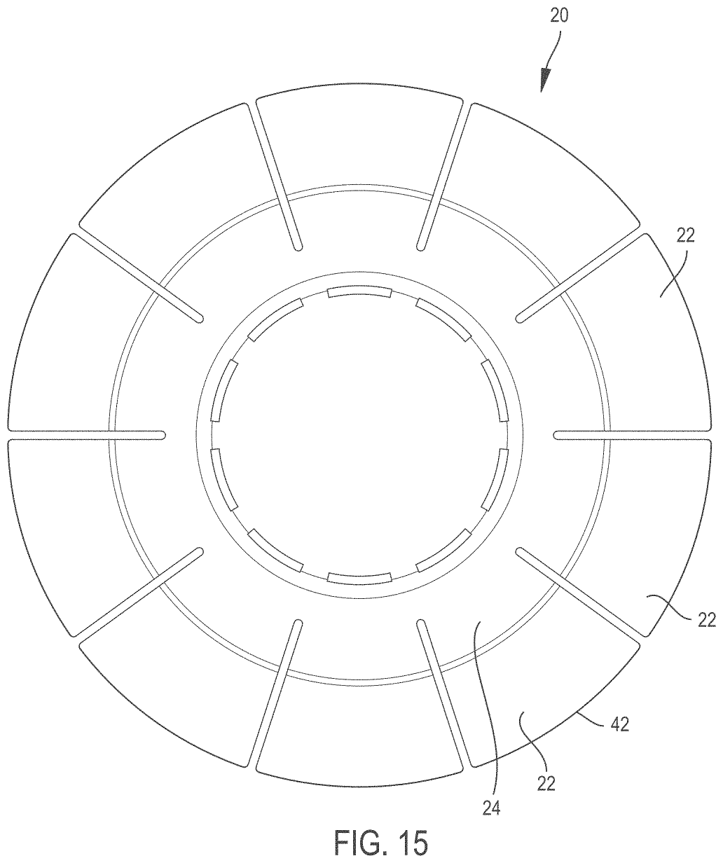

In other embodiments, the packet 208 may be or include a bias member. In some embodiments, the packet 208 may be a foam adhesive material sitting atop the reservoir 12. In such examples, when a user pushes down on the delivery device 10 (once the delivery device 10 is affixed to the skin surface), the foam adhesive may function like a spring that helps limit maximum pressure applied to the reservoir 12. The foam adhesive may also facilitate even distribution of pressure across the top of the reservoir 12. Assembly of the components described in connection with the above embodiments is described below with reference to FIGS. 49-61B and FIGS. 12-29B

Referring now to FIGS. 12-14, an exemplary delivery device 10 is depicted. The example delivery device 10 is shown in a storage state in FIGS. 12-14. As shown, the delivery device 10 may include a main body 20 and a reservoir 12. The reservoir 12 may include at least one delivery sharp 72. The delivery sharp 72 may be included on a sharp bearing body 26 which may be coupled to a wall of the reservoir 12. The main body 20 of the example delivery device 10 may have a round (e.g. circular) foot print and may include a central region 28 and a peripheral region 30. The central region 28 may be a raised region of the main body 20 and the peripheral region 30 may be a substantially flat region of the main body 20 which surrounds the central region 28. The thickness of the main body 20 may be substantially uniform over the entirety of the main body 20. The main body 20 may be formed as a thin sheet or disc of material which may be thermoformed to create the raised central region 28 and flat peripheral region 30.

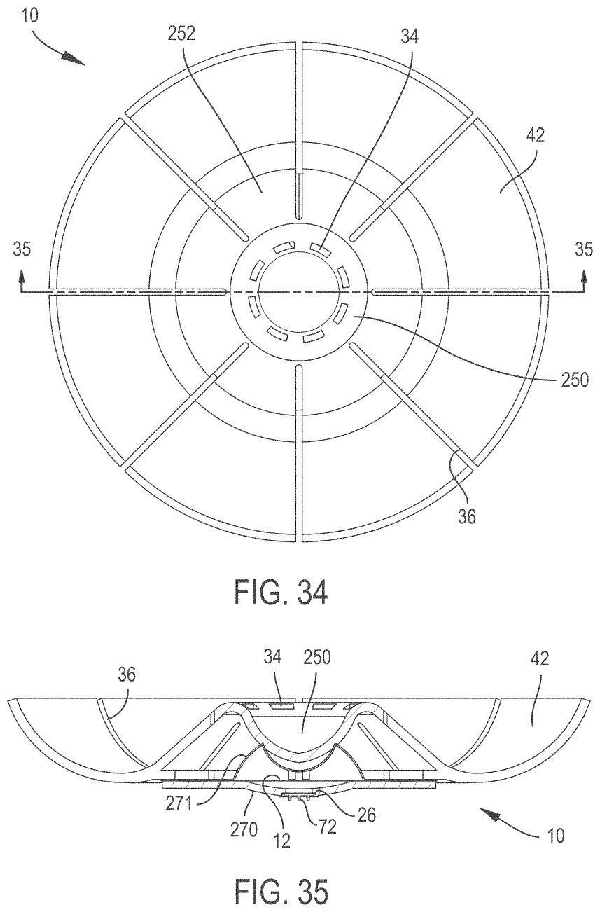

Alternatively, the main body 20 may be injection molded and the raised central region 28 and flat peripheral region 30 may be formed in the molding operation. In various embodiments where delivery devices 10 are or may be injection molded (e.g. the embodiments described in relation to FIGS. 12-35) the main body 20 may be injection molded so as to be in the storage state or in the delivery state. The main body 20 may transition more easily into the state in which it was molded from the opposite state. Thus, to lower the effort needed to transition a delivery device 10 from a storage state to a delivery state, it may be desirable to mold the main body 20 of the delivery device 10 in its delivery state configuration. During assembly of a delivery device 10, the main body 20 may be brought into its storage state configuration and remain in that configuration until use.

The central region 28 may be domed and the domed shape may establish a receptacle 32 on the proximal side of the main body 20 within which the reservoir 12 may be disposed. The reservoir 12 may be coupled within the receptacle 32 via adhesive or in another suitable manner. The central region 28 may also include a series of fenestrations 34 which may form a fenestrated ring in the central region 28. In the example, the fenestrations 34 are evenly spaced from one another and arranged in a circle which is generally coaxial with the center of the central region 28. In alternative embodiments, fenestrations 34 may be irregularly spaced or omitted. Additionally, in some embodiments, the fenestrations 34 may instead be replaced with thinned regions or a ring where the material of the main body 20 is thinned.

The main body 20 may include a number of slots 36. The slots 36 may extend from a peripheral edge 38 of the main body 20 toward a center or midpoint of the main body 20. In the example embodiment, the slots 36 extend in a radial direction. The slots 36 may extend through the entirety of the peripheral region 30. In some embodiments, and as shown, the slots 36 may additionally extend through at least a portion of the central region 28 as well. The fenestrations 34 in the central region 28 may be disposed radially inward of the terminus 40 of each of the slots 36. The main body 20 may thus include a central region 28 which is circumscribed by a number of petal members 42 which are spaced apart via the slots 36.

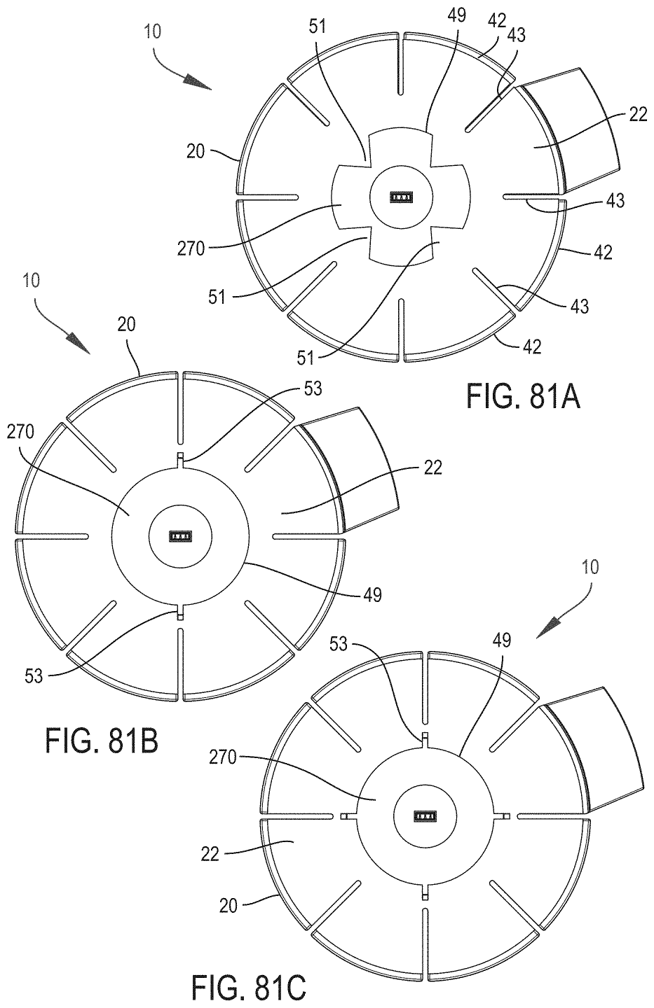

Referring now to FIG. 15, a plan view of the proximal face 24 of the main body 20 is depicted. As shown, adhesive 22 may be included on at least a portion of the proximal face 24. The adhesive 22 may be a skin compatible adhesive and may serve to couple a delivery device 10 to a skin surface at an infusion site. In the example embodiments, adhesive 22 may be included on the peripheral region 30 of the main body 20. Though adhesive 22 is shown covering the entire surface of each of the petal members 42 in the peripheral region 30, other embodiments may differ. For example, only certain petal members 42 may include the adhesive 22. In such embodiments, adhesive 22 may be included on at least one pair of oppositely disposed (e.g. diametrically opposed in the example embodiment) petal members 42. Only a portion (e.g. a majority of the surface area) of each petal member 42 included in the peripheral region 30 may be covered with adhesive 22 in some examples. Alternatively or additionally, the adhesive 22 may differ from petal member 42 to petal member 42. Certain petal members 42 may be covered with a more aggressive adhesive 22 while other petal members 42 may be covered with a less aggressive adhesive 22. In certain examples, the entirety of the proximal surface 24 may be covered in adhesive 22. Additional adhesive members 22 are described elsewhere herein (see, e.g., FIGS. 81A-C) and may be used on a delivery device 10.

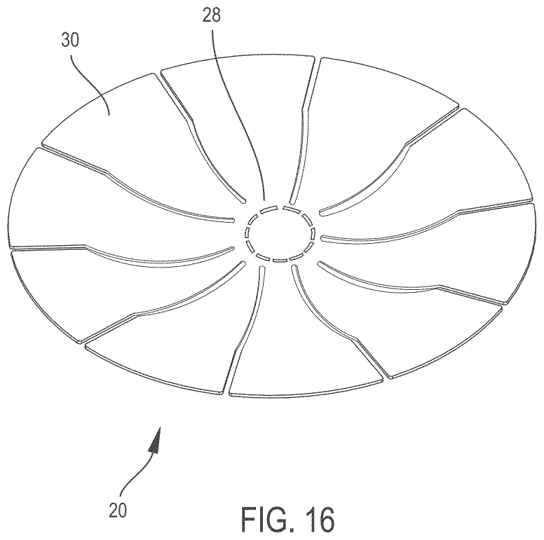

Referring now to FIG. 16, a conceptual representation of a main body 20 of a delivery device 10 is depicted in a delivery state. In the delivery state, at least the central region 28 of the main body 20 may substantially invert. The fenestrations 34 may facilitate this inversion by helping to allow for increased deflection of the main body 20 at the fenestrations 24. Thus, in place of a convex dome-like shape, the center region 28 of the main body 20 may take on a concave shape. As the peripheral region 30 is coupled to the center region 28, the peripheral region 30 may displace as a result of the inverting of the center region 28. In the example embodiment, the entire main body 20 takes on a bowl shape when transitioned to the delivery state. The peripheral region 30 may also spreadingly displace for at least a portion of the transition. The slots 36 in the main body 20 may help to facilitate spreading displacement of the petal members 42 as the transition takes place, thereby enhancing stretching of the skin of the user.

The main body 20 may be a bi-stable element or include at least one bi-stable region which may be stable in both the storage state and the delivery state. When an axial load is applied on the central region 28 and the main body 20 is in the storage state, the main body 20 may deform into an unstable state. The main body 20 may then exhibit a snap through buckling action which rapidly shifts the main body 20 into the stable delivery state similar to that shown in FIG. 16. Thus, only a triggering force may be applied to initiate the transition. The rest of the shift between the storage and delivery state may be caused by the snap through phenomenon.

FIG. 17 depicts a delivery device 10 in a storage state and adhered to skin 44 via adhesive 22 on a proximal face 24 of the main body 20. FIG. 18 is a conceptual representation depicting a delivery device 10 in the delivery state. As shown, the delivery device 10 may be applied to the skin 44 in the storage state. The delivery device 10 may then be transitioned to the delivery state. A spreading displacement of opposed petal members 42 of the main body 20 may occur as the transition transpires.

Two opposing points 46A, B disposed at the peripheral edge of the proximal surface 24 are shown in FIG. 17 and FIG. 18. When the delivery device 10 is in the storage state (FIG. 17), the shortest distance between the opposing points 46A, B is a straight line which does not pass through the proximal surface 24. This straight line is roughly parallel to the surface of the skin 44. In the delivery state, however, the shortest distance between the opposing points 46A, B is a straight line which passes through the proximal surface 24. As the skin 44 is fixed to the main body 20 via the adhesive 22 and cannot pass through the main body 20, the skin 44 may be forced to conform to the curvature of the proximal surface 24. Thus, the length of the skin 44 surface between the two points 46A, B when the delivery device 10 is in the delivery state may be greater than the length of the skin 44 surface between the points 46A, B when the delivery device 10 is in the storage state. The skin 44 may be placed under tension and stretched to accommodate this change in length. This stretching may, in turn, aid in facilitating puncture of the skin 44 by the delivery sharp(s) 72.

Due to the elasticity of the skin 44, the skin 44 may exert a restoring force against the proximal surface 24 of the main body 20 as it attempts to revert to an unstretched state. The main body 20 may resist this restoring force and retain its bowl shape. The reservoir 12, however, may be compressed between the skin 44 and the main body 20. This may aid in ensuring the delivery sharp(s) 72 puncture the skin 44 and enter fluid communication with a target delivery destination in the patient. Additionally, since the reservoir 12 may be collapsible, the restoring force exerted by the skin 44 may pressurize the reservoir 12 and urge fluid to pass out of the reservoir 12 via the delivery sharp(s) 72. Thus, the restoring force exerted by the stretched skin 44 may serve to empty and collapse the reservoir 12.

As mentioned above, in certain examples, some petal members 42 may not include adhesive 22 regions or may have a proximal surface 24 which is at least partially covered in adhesive 22 that is less aggressive than adhesive 22 of on other petal members 42. In embodiments where some petal members 42 are devoid of adhesive 22, this may help to limit stretching of the skin 44 Likewise, petal members 42 with less aggressive adhesive 22 may release the patches of skin 44 to which they are affixed if force needed to stretch the skin 44 exceeds a threshold. The petal members 42 themselves may also be constructed such that at least one of the petal members 42 includes a relief region (e.g. a thin or narrow region). For example, if force needed to stretch the skin 44 is above a threshold, one of more of the petal members 42 may bend or buckle at the relief region to relieve some of the tension on the skin 44.

This may be desirable as it may help to mitigate potential discomfort during an injection due to excessive tensioning of the skin 44. Additionally, this may be helpful in certain patient populations as skin characteristics vary significantly with age, hydration state, lifestyle (sun exposure, nutrition), etc. It may be desirable that slacker or looser skin be stretched to a greater degree than highly elastic skin. Thus, instead of providing a variety of delivery devices 10 with different adhesives 22 targeted at specified patient populations, a delivery device 10 may be made in a more universal manner.

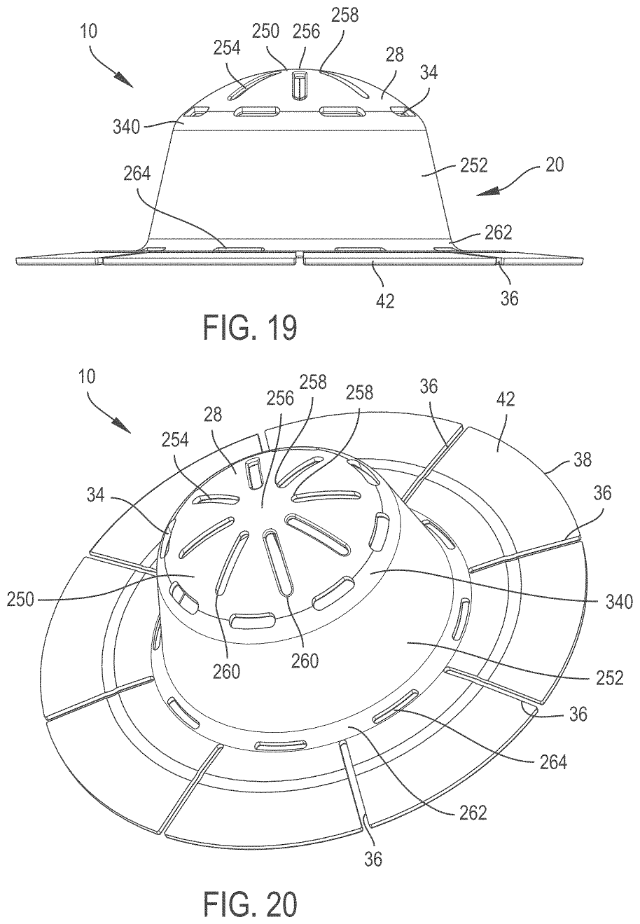

With reference to FIG. 19 and FIG. 20, in another embodiment, a delivery device 10 may include a central region 28 having a top surface 250 and a supporting structure 252 integral with the top surface 250. The supporting structure 252 may have a round, e.g., substantially circular, base 262. The peripheral region 30 may be roughly annular shaped and may include an inner perimeter coincident with the base 262 and an outer perimeter, or peripheral edge 38. The delivery device 10 may be constructed of a nylon material such as Nycoa 2012 nylon or other, similar nylon materials, and may be formed by injection molding. Any other suitable plastic may be used. The top surface 250 may have a round footprint, e.g., of roughly circular shape, and may be convex, forming a dome shape. The top surface 250 may have a periphery 340. The top surface 250 may include slots 254. The slots 254 may be cutouts, apertures, holes, openings, or voids in various embodiments. The slots 254 may help the delivery device 10 to transition from the storage state to the delivery state with reduced pressure from above. The slots 254 may extend radially with respect to a center point 256 of the top surface 250 such that their respective first endpoints 258 surround a region including the center point 256 of the top surface 250 and their respective second endpoints 260 may each terminate a distance (e.g., the slots 254 may each terminate the same distance) from the periphery 340 of the top surface 250. In embodiments including slots 254, the slots 254 may be disposed at regular angular increments (though need not be). In embodiments described herein including slots 254, the slots 254 may (though need not necessarily be) each be of the same length.

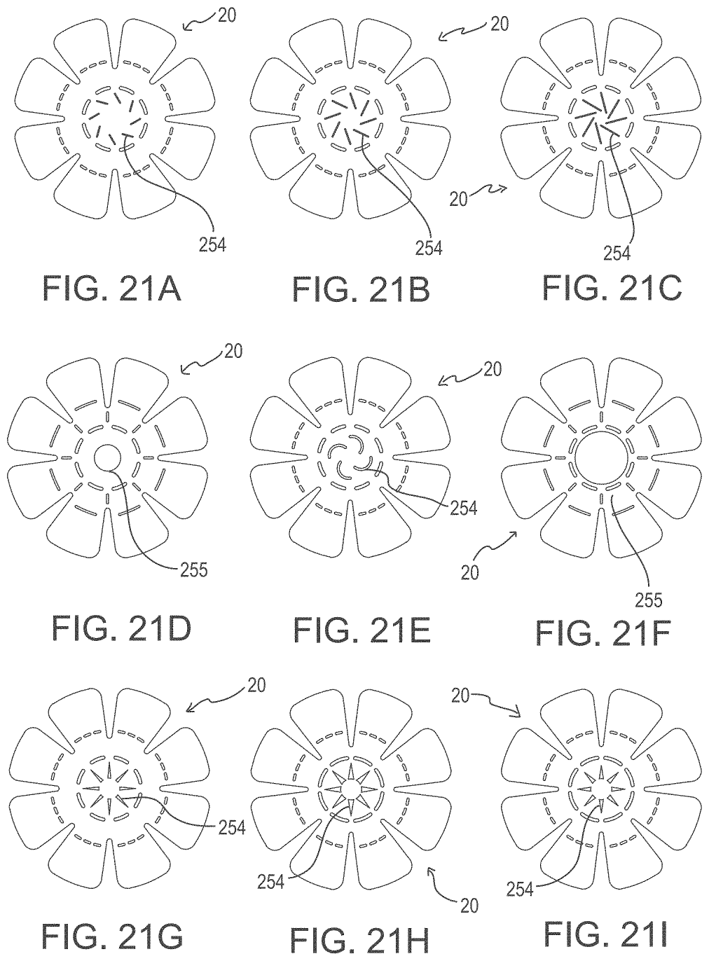

Referring now to FIGS. 21A-21I, a variety of different main body 20 embodiments are depicted. The exemplary main bodies 20 are shown in a flat state and may be thermoformed into a configuration such as that shown in, for example FIG. 19. Though thermoformable main bodies 20 are depicted, the features described in relation to thermoformed main bodies 20 may be included in main bodies 20 which are manufactured in any desired manner. As shown in FIGS. 21A-21I, the slots 254 may be provided in a number of different formats. Additionally, in some embodiments, slots 254 may not be included.

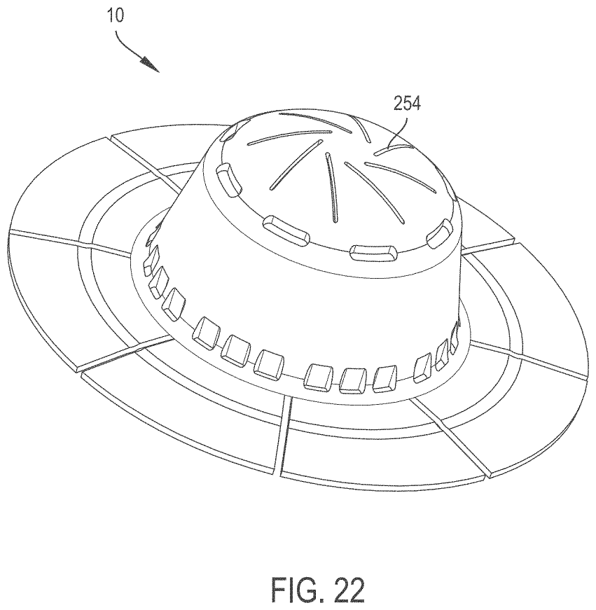

In some embodiments, and as also shown in FIG. 22, the slots 254 could be disposed such that they do not extend radially with respect to the center point 256. For example, the slots 254 may each extend at a common angle with respect to a radial direction. In such embodiments, slots 254 may be evenly spaced about the top surface 250 and may each be of the same length. In other embodiments, the slots 254 may not all extend at a common angle to the radial direction. At least one of the slots 254 (and perhaps all) may be disposed at a different angle to the radial direction. In some embodiments, the slots 254 may be relatively short, positioned about the periphery 340 of the top surface 250, and may be disposed within an outer region of the top surface 250 (see, e.g., FIG. 21A). In other embodiments, slots 254 may extend across an outer region and intermediate region of the top surface 250 (see, e.g., FIG. 21B). In still other embodiments, slots 254 may extend from the outer region of the top surface and into a center region of the top surface 250 (see, e.g., FIG. 21C). The angled slots 254 may aid in lowering the amount of pressure needed to transition the delivery device 10 from a storage state to a delivery state. Positioning the slots 254 at a sharper angle with respect to the radial direction may generally lower this pressure. The width of the slots 254 may slightly decrease during at least a portion of the transition from the storage state to the delivery state.

In other embodiments, and referring primarily to FIG. 21E, at least one of the slots 254 may have a curvature. The curvature may be defined by a constant or variable radius. The curvature may only be present over a segment of the slot 254. In alternative embodiments, a slot 254 may include two or more sections which are angled with respect to one another. In the example embodiment shown in FIG. 21E, four curved slots 254 are shown and are spaced apart at even angular increments. The slots 254 are arcuate and include a first end 258 and second end 260. Each example slot 254 is oriented so as to initially begin extending in a first direction from the first end 258 and curve so as to extend in a second direction as the slot 254 reaches the second end 260. The second direction may be closer to perpendicular (or may be perpendicular) to the radial direction than the first direction.

In some examples, and referring now primarily to FIG. 21D and FIG. 21F, the top surface may not include a slot 254 or slots 254 but may instead include at least one aperture 255. In the examples shown, the aperture 255 is disposed centrally within the top surface 250. The aperture 255 may extend over a minority or a majority of the top surface 250. In some embodiments, the aperture 255 may encompass nearly the entirety of the top surface 250.

As shown exemplarily in FIG. 21D and FIG. 21F, slots 254 may also be included in other regions of a main body 20. In the example embodiment, the region of the main body 20 which would become the supporting structure 252 (when the main body 20 is thermoformed) includes slots 254. These slots 254 may be straight, curved, angled (with respect to the radial dimension) or some mix thereof as with various top surface 250 slot 254 patterns described herein. As shown, the slots 254 are spaced at regular angular intervals and are spaced between petal members 42 of the main bodies 20.

In still other embodiments, the width of one or more of the slots 254 may vary over the length of that slot 254. A number of embodiments including variable width slots 254 are depicted in FIGS. 21G-21I. The slots 254 may change in width in a continuous manner and may terminate with a pointed first or second end 258, 260. Variable width slots 254 may extend along a radial direction, though need not necessarily do so in all embodiments. In the example embodiments, each of the slots 254 are widest proximal to the center point 256 of the top surface 250 and continuously decrease in width as the slot 254 extend distally toward the periphery 340 of the top surface 250. Thus, each of the top surfaces 250 depicted in FIGS. 21G-21I have a sunburst type pattern of slots 254. In other embodiments, the slots 254 need not necessarily continuously increase or decrease in width from one end to the other.

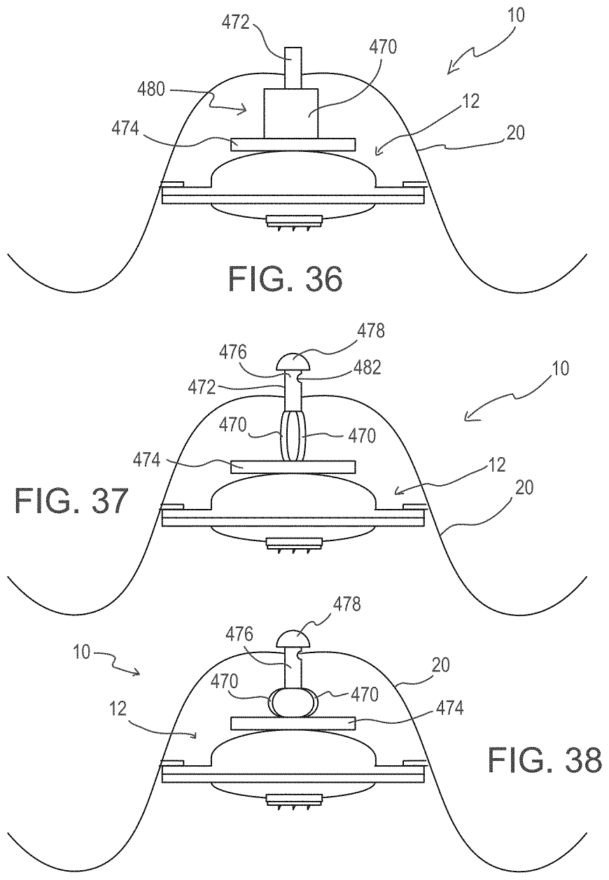

Still referring to FIG. 22, the central region 28 may be monolithically formed with the petal members 42 comprising the regions between respective pairs of slots 36 (see also FIGS. 12-18 and the examples and embodiments described above with respect thereto). The supporting structure 252 may extend upward from the petal members 42 at a 90° angle or an angle greater than 90 degrees, e.g., 100-105 degrees, although the angle measure need not be limited to a range. The distance (vertically) from the base 262 of the supporting structure 252 to the periphery 340 of the top surface 250 may be long enough to provide a receptacle in the central region 28 for a reservoir 12 (see, e.g., FIG. 60) and, in some embodiments, any packets 208 and/or containers 350 (see, e.g., FIGS. 10A-11), springs, or foam adhesive material. The receptacle may also be sized to house portions of an actuation assembly or dispensing assembly 480 (see, e.g., FIG. 36). As described in greater detail elsewhere herein, packets 208 may include gas bladders, butane packets, or delivery force supplying packets and any associated containers 350 such those described above with respect to FIGS. 10A-11. In some embodiments the aforementioned distance may be approximately 0.3 inches (e.g. 0.315 in). The slots 36 may extend from the peripheral edge 38 of the delivery device 10 to the base 262 of the supporting structure 252, but may terminate at the base 262 and not extend into the supporting structure 252 itself. In such embodiments, rather than the entire central region 28 substantially inverting when pressure is applied from the top (e.g., by a finger), only the top surface 250 may invert, taking on a concave shape in the delivery state. The supporting structure 252 may in some embodiments include fenestrations 264 evenly spaced about the base 262. The fenestrations 264 may facilitate manufacturing of the delivery device 10 in embodiments in which the main body 20 is thermoformed.

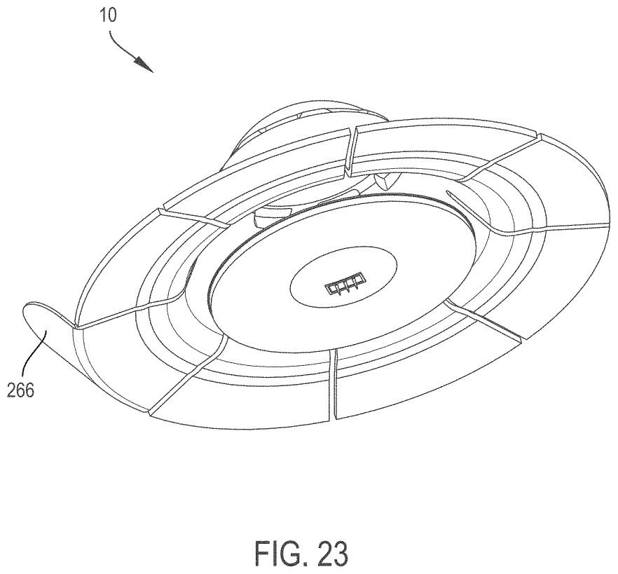

In some embodiments and as shown in one example in FIG. 23, at least one of the petal members 42 may be made of an extended length such that an outward end of the petal member 42 may be operated by a patient or health care provider as a pull tab 266. The pull tab 266 may be grasped by a user to remove the delivery device 10 from the skin after use. The pull tab 266 may be of any suitable shape. In an example, the pull tab 266 is approximately semicircular in shape, with a first, rounded end and a second end opposite the first end, attached to a petal member 42. The second end may be attached or formed integral with the petal member 42 by injection molding or any other known technique that permits the pull tab 266 to be lifted sufficiently from the skin surface to be held by a user.

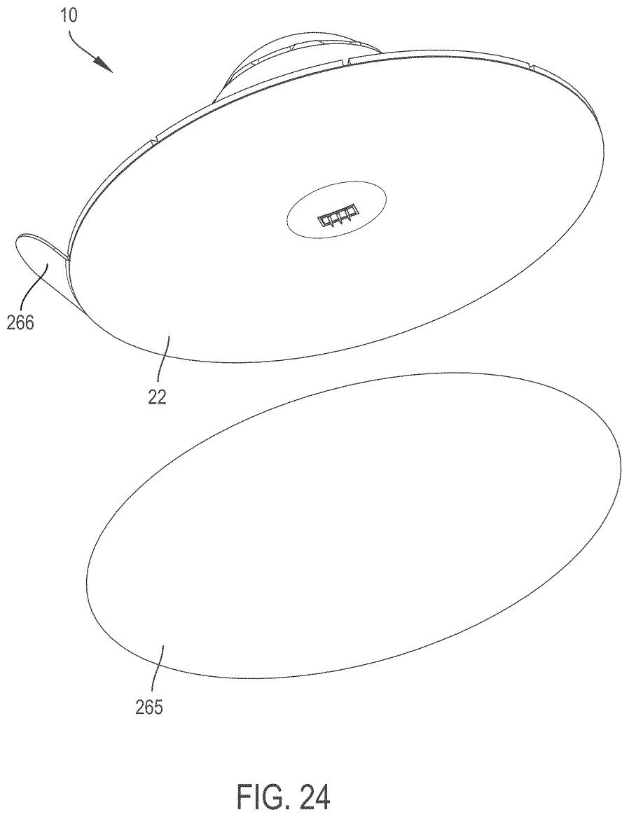

Referring now to FIG. 24, the pull tab 266 may also serve to facilitate a user peeling off a release liner 265 from the bottom of the delivery device 10 before the delivery device 10 is applied to the skin surface via an adhesive 22. A release liner 265 may be removed in a manner similar to how a release liner is peeled from a bandage before application to skin. An example delivery device 10 having a pull tab 266 and including release liner 265 and adhesive 22 is depicted in FIG. 24. The release liner 265 is exploded away from the adhesive 22 for illustrative purposes.

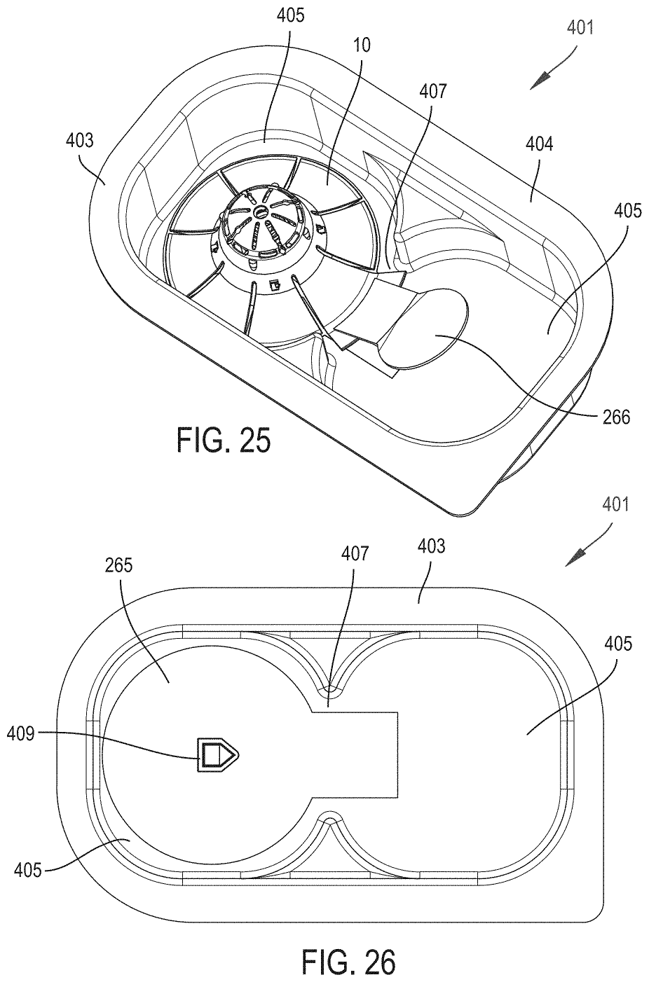

Referring now to FIGS. 25-26, an example embodiment of a package 401 for a delivery device 10 is depicted. The delivery device 10 is depicted in place within the package 401 in FIG. 25. The package 401 may include a first component 404 and a second component 406 (not shown in FIGS. 25-26, see, e.g., FIG. 104). The first component 404 may be a rigid component such as a plastic. The second component 406 may be a flexible component (e.g. EtOx permeable sheet) which may be peeled from the first component 404 to access the delivery device 10. The interior of the package 401 may be a protected environment (e.g. sterilized via EtOx or any other suitable manner) until the second component 406 is removed from the first component. The second component 406 may releasably couple to a rim 403 included on the first component.

The first component 404 may include one or more wells 405. The delivery device 10 may be disposed in one of the wells 405. The pull tab 266 of the delivery device 10 may project along a passage 407 connecting the two wells 405. To remove the delivery device 10 a user may reach into the well 405 unoccupied by the delivery device 10 to grasp a portion of the pull tab 266 extending into that well 405. The delivery device 10 may then easily be lifted out of the package 401 by pulling on the pull tab 266.

As shown in FIG. 26, the liner 265 for the adhesive 22 (see, e.g., FIG. 24) may be coupled to the surface of one of the wells 405. This may aid in limiting any movement of a delivery device 10 within a package 401 during transport and handling. As the delivery device 10 is removed, the liner 265 may remain behind in the package 401. Thus the delivery device 10 may be rendered ready for use when removed from a package 401. A cap or cover 409 (e.g. receptacle that surrounds the delivery sharp(s) 72) for the delivery sharp(s) 72 of the delivery device 10 may be included in some examples. The cover 409 may also be coupled to the package 401 and stay behind when the delivery device 10 is removed from the package 401. Again, this may aid in rendering a delivery device 10 ready for use when taken out of the package 401.

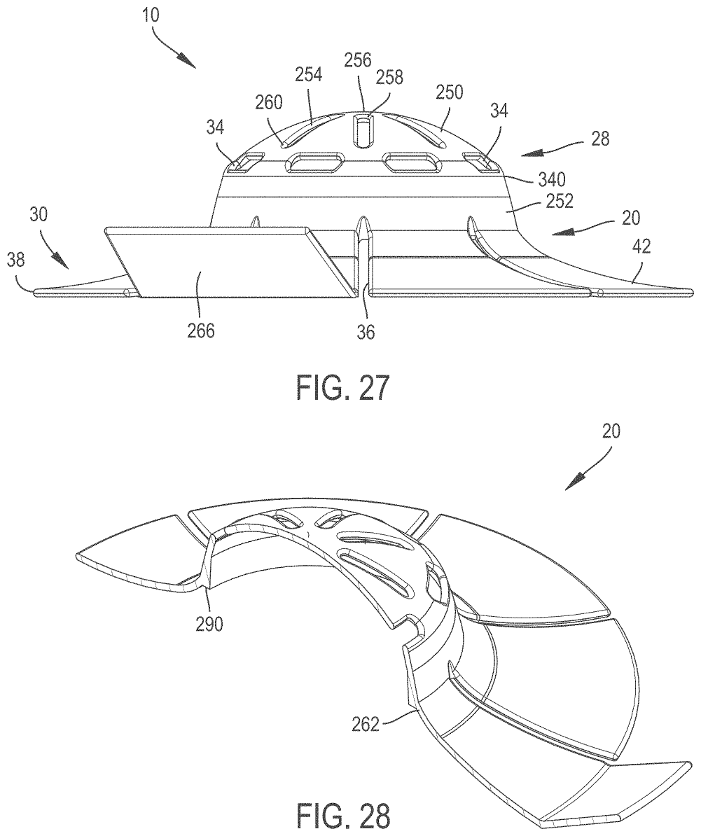

With reference to FIGS. 27-28 and FIGS. 30A-30B, in some embodiments, delivery devices 10 may include a central region 28 which is roughly thimble, or dome, shaped but has a relatively shorter height compared to certain other embodiments described herein. The distance (vertically) from the base 262 to the periphery 340 of the top surface 250 may be relatively shorter. In some embodiments, the aforementioned distance may be approximately 0.15 inches.

Additionally or in the alternative, the peripheral region 30 may not be a substantially flat annular shape. The peripheral region 30 may be defined by curved petal members 42 that continue in a downward direction such that their peripheral edge 38 is spaced from the plane of the base 262 of the supporting structure 252 (e.g. about the same or less than the distance from the base 262 to the periphery 340 of the top surface 250). The peripheral edge 38 may be disposed along a plane which is more distal to the periphery 340 of the top surface 250 than the base 262. As depicted in FIG. 27, the delivery device 10 is shown in the storage state. The delivery device 10 may include slots 36 which may be disposed between the petal members 42 like other delivery device 10 embodiments described herein. An adhesive 22 (see, e.g., FIG. 24) may be affixed to at least a part of at least two of the petal members 42.

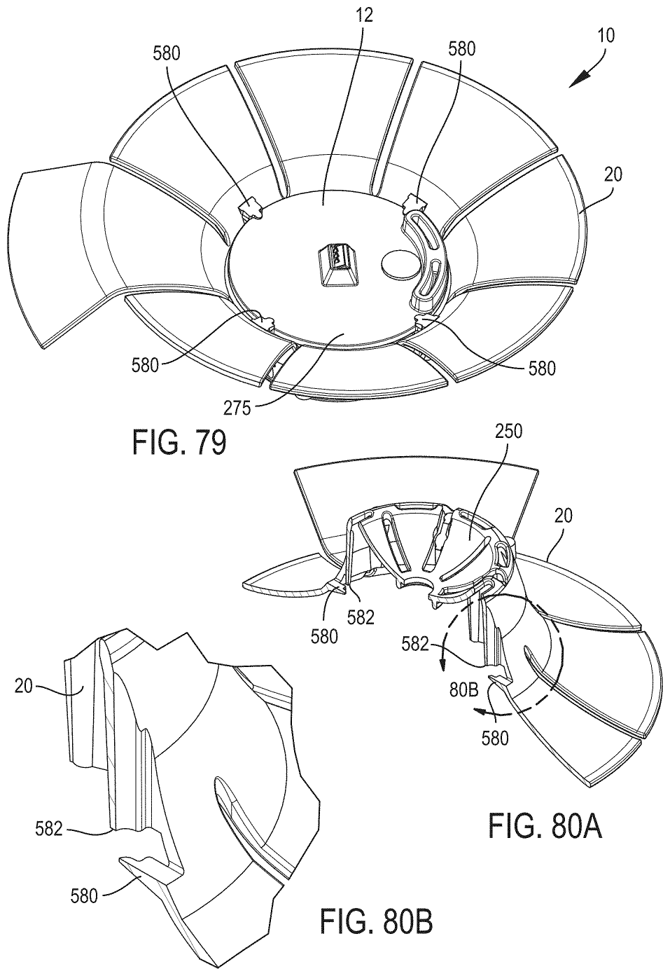

As best shown in FIG. 28, a perspective cross-sectional view of a main body 20 of a delivery device 10, the main body 20 may include an interior ridge 290. The ridge 290 may be disposed at the base 262 of the supporting structure 252. The supporting structure 252 may be thickened in a region near the base 262 so as to create the ridge 290. This may allow for the ridge 290 to be formed easily in, for example, an injection molding operation which forms the rest of a main body 20. This may also provide extra rigidity to the supporting structure 252. The ridge 290 may provide a step, ledge, or other mounting surface upon which a portion of a reservoir assembly 12 of a delivery device 10 may be mounted. Such a ridge 290 may be included in any of the delivery device 10 embodiments described herein. Reservoir assemblies 12 and ridges 290 are further described elsewhere in the specification. Any embodiments including ridges 290 may alternatively include retention tabs 580 and stop surfaces 582 such as those shown in FIGS. 79-80B.

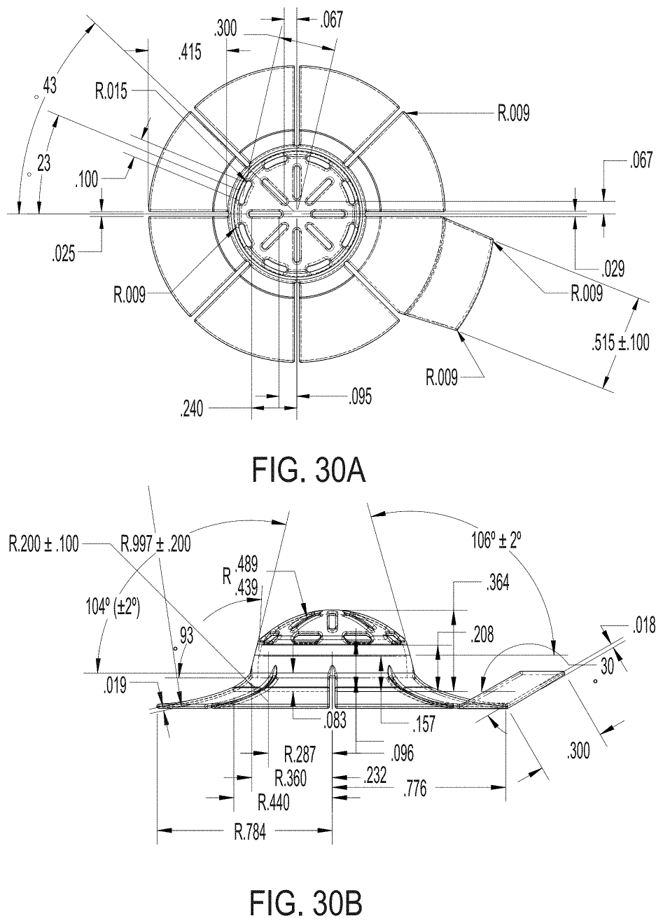

Referring now primarily to FIGS. 30A-30B, two conceptual representations of a delivery device 10 transitioning from a storage state to a delivery state are shown. When the delivery device 10 is affixed to the skin with an adhesive 22 and pressure is applied to the delivery device 10 from above, e.g., by a user's fingertip, the delivery device 10 may transition to a delivery state. When the petal members 42 are pushed against the surface of the skin, the petal members 42 may spreadingly displace outward and the skin and/or patient's body may force at least a portion of the petal members 42 to curl upward. In turn, this may cause the skin to stretch as parts of opposing petal members 42, each affixed to the skin surface by adhesive 22 (only shown in FIG. 30A), move apart from one another or spreadingly displace. As the delivery device 10 transitions to a delivery state, at least a portion of each of the curved petal members 42 may curve further or with a tighter radius of curvature. When a delivery state is reached, the curvature of the petal members 42 may be such that they may extend from the base 262 to an inflection point 360. The inflection point 360 may fall in a plane spaced from that of the base 262 and in such embodiments may also be referred to as a lowest point. In such embodiments, the lowest point 360 may be in a plane more distal to the periphery 340 of the top surface 250 than the base 262. From the inflection point 360, the petal members 42 may curve back upward so as to become increasingly more proximal to the plane in which the periphery 340 of the top surface 250 is disposed. The peripheral edge 38 of the petal members 42 may, for example, be disposed at a point at or above (more proximal the plane of the periphery 340 of the top surface 250) the plane of the base 262. The petal members 42 may, though need not necessarily, each have a constant radius of curvature from the inflection point 360 to the peripheral edge 38. The constant radii curvature back upward may enhance capability of the petal members 42 to curl upward. This may in turn enhance stretching of the skin of the user as points 360 on opposing petal members 42 (each affixed to the skin by adhesive 22) spreadingly displace. As mentioned elsewhere herein, the top surface 250 of the main body 20 may also invert as the delivery device 10 is transitioned to the delivery state 10.

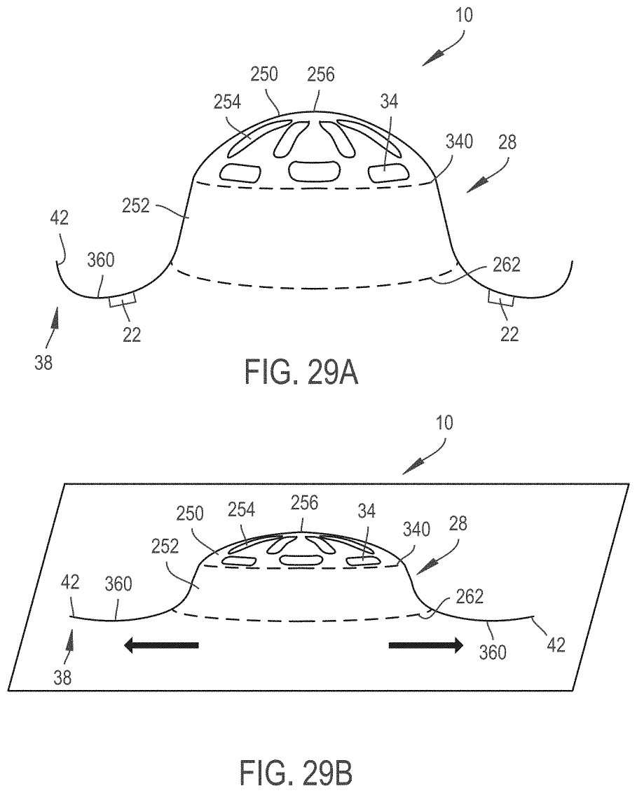

In some non-limiting examples, a delivery device 10 may have dimensions and radii of curvature as shown in FIGS. 29A-29B when in a storage state. It is to be understood that the dimensions shown are merely exemplary. Other delivery devices 10 of different size and having the same proportions are possible and contemplated. Additionally, delivery devices 10 with different dimensions and proportions are possible and contemplated.

Referring to FIGS. 27-28 and FIGS. 30A-30B, in some embodiments, the supporting structure 252 may not include fenestrations 264 (see, e.g. FIG. 19) evenly spaced around the base 262. The main body 20 may be manufactured by injection molding. Those of skill would readily appreciate that other manufacturing techniques could be used. The main body 20 may be constructed of one monolithic piece of material such that the central region 28 and peripheral region 30 are integral with respect to each other. The main body 20 may be constructed of a polymer material. In some embodiments, the main body 20 may be a nylon material such as Nycoa 2012 nylon or other, similar nylon materials. In other embodiments the main body 20 may be made of a polypropylene material. The main body 20 may be manufactured of a material that serves to minimize water absorption, or a material that serves to maximize capacity to stick to an adhesive 22. A material that achieves both of these objectives to any desired degree for each may be selected. These materials may be used for any of the main bodies 20 described herein.

Still referring to FIGS. 27-28 and FIGS. 30A-30B, the top surface 250 may have a round footprint, e.g., be of roughly circular shape, and may be convex, forming a dome shape (including the periphery 340). The top surface 250 may include slots 254. The slots 254 may be cutouts, apertures, holes, openings, or voids in various embodiments. The slots 254 may extend radially with respect to a center point 256 of the top surface 250 such that their respective first endpoints 258 surround a region including the center point 256 of the top surface 250 and their respective second endpoints 260 may each terminate a distance (e.g., the slots 254 may each terminate the same distance) from the periphery 340 of the top surface 250. In certain embodiments, the slots 254 may be disposed at regular angular increments and each be of equal length (though this need not be true to all embodiments).

Still referring to FIGS. 27-28 and FIGS. 30A-30B and as described above with reference to FIG. 22, the slots 254 could, in an alternative embodiment, be disposed such that they do not extend radially with respect to the center point 256. For example, the slots 254 may each extend at a common angle with respect to the radial direction. In such embodiments, slots 254 may be evenly spaced about the top surface 250 and may each be of the same length. In other embodiments, the slots 254 may not all extend at a common angle to the radial direction. At least one of the slots 254 (and perhaps all) may be disposed at a different angle to the radial direction.

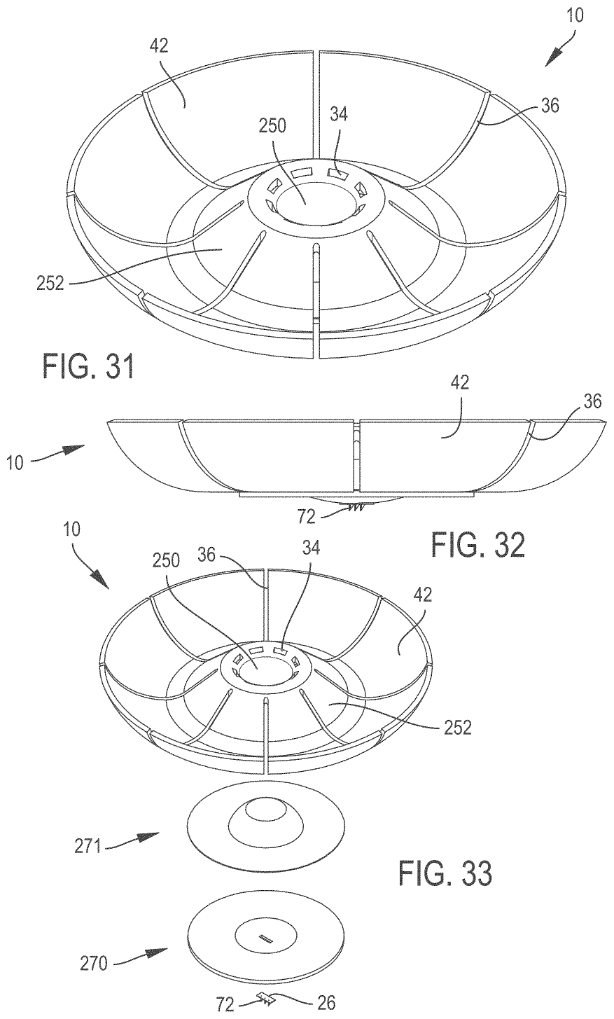

Referring now primarily to FIGS. 31-35, a number of views of a conceptual representation of a delivery device 10 in a delivery state are shown. As described above (and also with reference to the embodiments of FIGS. 12-20), the delivery device 10 may, upon downward pressure being applied to the top surface 250, transition from a storage state to a delivery state in which the main body 20 of the delivery device 10 is substantially, or at least partially, inverted. A user may remove an adhesive liner 265 (see, e.g., FIG. 24) from the delivery device 10 and apply the delivery device 10 to the skin. The user may then press downward (i.e., toward the skin) on the top surface 250. This may cause the petal members 42 to spreadingly displace outward and curl upward (over at least a portion thereof), stretching the skin. The top surface 250 may invert, driving delivery sharp(s) 72 into the skin, and remain inverted when the delivery device 10 attains the delivery state. The peripheral region 20 may also take on an inverted shape due to the curling of the petal members 42.

In various embodiments, certain regions of the main body 20 of the delivery device 10 may remain static or may not invert. Thus, a main body 20 may include inverting regions and resilient regions. Though described as resilient regions, it is to be understood that some bending or deformation may still occur as pressure is applied. These regions may, however, appear generally similar or extend/project in the general same direction in both the storage and delivery state. As shown, the peripheral region 30 and top surface 250 may invert, but a portion of the central region 28 may resist deformation to this degree. The supporting structures 252 shown in other embodiments described herein (see, e.g., FIG. 19 or FIG. 27) may also be a resilient region. Thus, certain delivery devices 10 may include a main body 20 with invertible regions which are separated from one another by a resilient region.

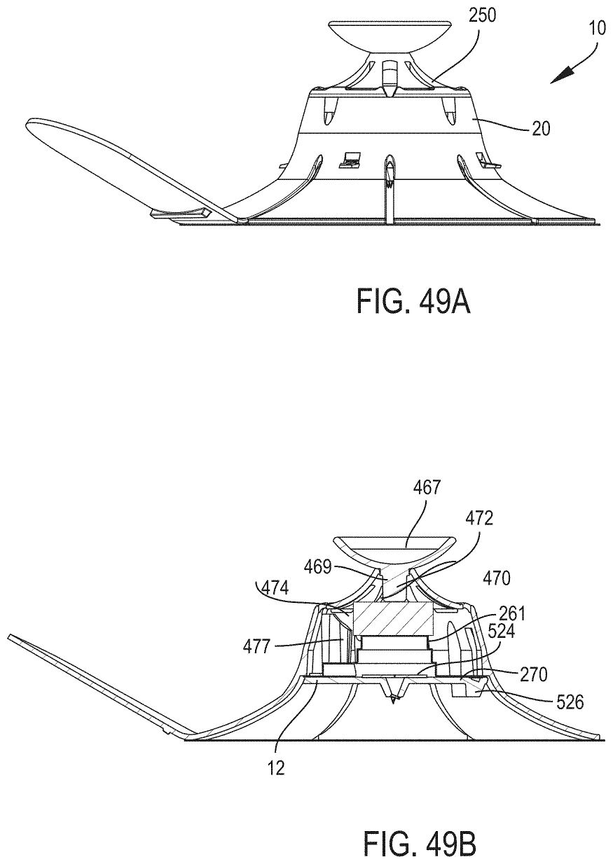

Still referring to FIGS. 31-35, the reservoir 12 may be formed as an assembly and may include a reservoir portion 271 and a holder 270 (described in greater detail below with reference to FIGS. 49-61B). A reservoir 12 may be compressed and/or at least partially collapsed so as to deliver a medical agent contained therein when the delivery device 10 is transitioned to the delivery state. The user may then remove the delivery device 10 from the skin. The slots 254 may aid the delivery device 10 to transition from the storage state to the delivery state with reduced pressure from above. The fenestrations 34 may also facilitate the transition. As described above with reference to the embodiments of FIGS. 12-20, there may be room in the central region 28 for a reservoir 12 and sharp bearing body 26 (see additional description with reference to the embodiments of FIGS. 12-20 and FIGS. 49-61B). In some embodiments, packets 208 and/or containers 350 (see, e.g., FIGS. 10A-11) and/or foam adhesive material may also be housed within the central region 28. As described in greater detail elsewhere herein, packets 208 may include gas bladders, butane packets, or delivery force supplying packets and any associated containers 350 such as those described above with respect to FIGS. 10A-11. In some examples, one or more petal members 42 may be constructed to incorporate a pull tab (not shown in FIGS. 31-35) such as the pull tab 266 described above with reference to FIG. 23.

Referring now to FIG. 36, a block diagram of an exemplary delivery device 10 is depicted. As shown, the delivery device 10 may include a main body 20 and a reservoir 12. The delivery device 10 may also include one or more bias member 470. The one or more bias member 470 may be included as part of a dispensing assembly 480 included in a delivery device 10. The dispensing assembly 480 may aid in applying pressure to the reservoir 12 and aid in expelling fluid from the reservoir 12 over the course of the injection. In some embodiments, the dispensing assembly 480 may include a depressor body 472 which may be coupled to or associated with the at least one bias member 470. The depressor body 472 may include or be coupled to (perhaps indirectly via the bias member 470) a reservoir interface member 474 which may also form part of a dispensing assembly 480 of a delivery device 10. In certain examples, a reservoir interface member 474 may be omitted and the bias member 470 may directly contact the reservoir 12.

In some embodiments, the bias member 470 may be in an unstressed state when the associated delivery device 10 is in a storage state. User interaction with the delivery device 10 to transition the delivery device 10 to a delivery state may involve applying pressure to the depressor body 472 of the dispensing assembly 480. This may displace the depressor body 472 in the direction of the reservoir 12. The depressor body 472 may include an engagement feature (e.g. catch or detent) which may engage with a retention feature of the delivery device 10 (e.g. one defined in the main body 20) to hold the depressor body 472 in the displaced position. Displacement of the depressor body 472 may in turn cause a bias to be stored in the bias member 470. With the delivery device 10 transitioned to the delivery state, the bias member 470 may restore to an unstressed state. As the bias member 470 restores, the reservoir interface member 474 of the dispensing assembly 480 may be urged against the reservoir 12 to collapse the reservoir 12 and drive fluid into a patient. Thus without, for example, sustained manual pressure against the delivery device 10, pressure may be applied to the reservoir 12 over a period of time sufficient to fully deliver contents of the reservoir 12 (e.g. 5 minutes in certain embodiments).

In other embodiments, the bias member 470 may be in a stressed state when the associated delivery device 10 is in a storage state and may be coupled to or associated with the depressor body 472 of the dispensing assembly 480. The depressor body 472 may interface with a portion of the delivery device 10 (e.g. the main body 20) so as to resist displacement under the restoring force exerted by the bias member 470. This may prevent the bias member 470 from restoring from its stressed state. A catch or detent in the depressor body 472 may, for instance, be in engagement with the main body 20 when the delivery device 10 is in a storage state. User interaction with the delivery device 10 to transition the delivery device 10 to a delivery state may disengage the depressor body 472 such that the depressor body 472 is free to displace. Once the depressor body 472 is free to displace, the bias member 470 may restore to an unstressed or at least less stressed state and drive the reservoir interface member 474 of the dispensing assembly 480 against the reservoir 12. Over a period of time, this may cause the reservoir 12 to collapse such that fluid is driven out of the reservoir 12 and into a patient.

Referring now to FIGS. 37-38, a representational example of a delivery device 10 which includes a bias member 470 that is unstressed in the storage state is depicted. The delivery device 10 may include a main body 20 and a reservoir 12 as with various other delivery devices 10 described above. As shown, the depressor body 472 of the dispensing assembly 480 may include an elongate member 476 such as a pin which may extend through the top surface 250 of the main body 20. In some embodiments, the elongate member 476 may include a head 478 or other surface at a distal end of the elongate member 476. The head 478 may include a rounded or tapered portion to aid in passing the head 478 though an aperture in the main body 20 during assembly. Opposite the tapered or rounded portion, the head 478 may define a step or ledge. The ledge of the head 478 may limit displacement of the elongate body 476 as the ledge may be unable to easily pass back through the aperture in the main body 20. An end of the elongate member 476 opposite the head 478 may couple to one or more bias member 470. The reservoir interface member 474 may be coupled to the one or more bias member 470 such that the one or more bias member 470 is disposed intermediate the elongate member 476 and the reservoir interface member 474. In the example embodiment, the one or more bias member 470 is depicted as a set of bow springs though any suitable number of bow springs may be used. In alternative embodiments, other bias members 470 may be used (e.g. resilient foam, coil spring, air bladder, rubber body, elastomeric body, etc.).

As pressure is applied to transition the delivery device 10 to the delivery state, the elongate body 476 may displace toward the reservoir 12. This may cause the bias members 470 to become stressed. As shown, the elongate body 476 includes a detent or notch 482. The notch 482 may engage with the main body 20 to hold the elongate body 476 in a depressed state. Engagement of the notch 482 with the main body 20 may also serve to indicate a delivery device 10 has been used.

With the elongate body 476 held in place, restoration of the bias members 470 to a less stressed state may drive displacement of the reservoir interface member 474 into the reservoir 12. As mentioned above, this may drive reservoir 12 contents out of the reservoir 12 and into the patient. It should be noted that, in various examples, at least some portion(s) of the main body 20 may spreadingly displace and/or invert as the delivery device 10 is transitioned to the delivery state (see, e.g., FIG. 31). This is not depicted in FIGS. 37-38 for ease of illustration.

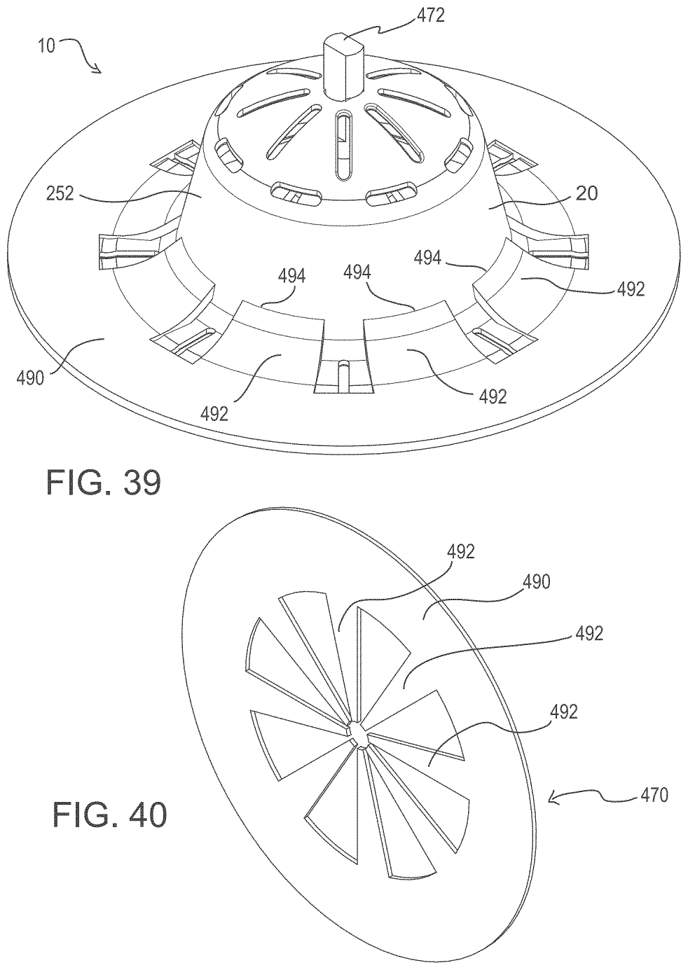

Referring now to FIGS. 39-40, in certain embodiments, a delivery device 10 may include a bias member 470 which is in a stressed state while the delivery device 10 is in a storage state. As shown in FIG. 40, a bias member 470 (depicted in an unstressed state) may include a peripheral body 490. The peripheral body 490 may for example, be annular in shape though any suitable shape may be used. A number of bias projections 492 may extend from the peripheral body 490 toward the center of the bias member 470. The bias projections 492 may extend radially inward toward the center of the bias member 470 from the peripheral body 490. In the example embodiment, the bias projections 492 may be spaced at even angular increments though need not be in all embodiments. The peripheral body 490 may be constructed of any suitable material and in some examples, may be a resilient plastic or a spring steel.

A main body 20 of a delivery device 10 may include a number of passages 494 which extend through the main body 20. The passages 494 may be positioned in a support structure 252 of the main body 20. The spacing of the passages 494 may correspond to the spacing of bias projections 492 on the bias member 470. When a delivery device 10 is assembled, the bias projections 492 may be introduced into and partially through respective passages 494 in the main body 20. The peripheral body 490 may rest on a distal face of the peripheral region 30 (see, e.g. FIG. 27) of the main body 20.

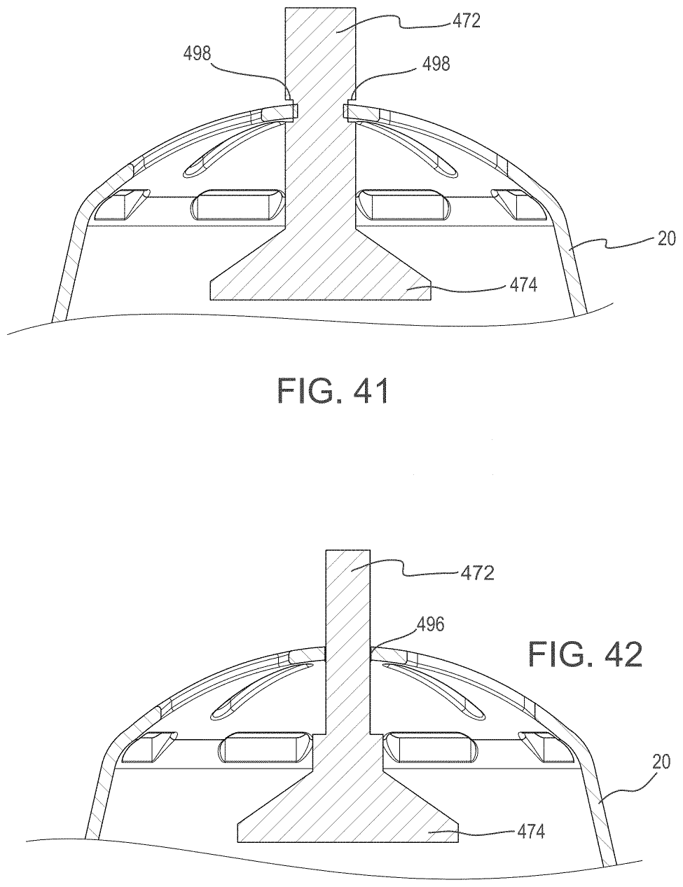

Referring now also to FIGS. 41-43, the delivery device 10 may include a depressor body 472. In the example shown, the depressor body 472 includes a reservoir interface member 474 at a proximal end thereof. The depressor body 472 may be rotationally displaceable within an aperture 496 in the main body 20. The aperture 496 may be disposed in the top surface 250 of the main body 20 as shown in FIG. 41 for instance. The depressor body 472 may be rotated from a translational displacement constraining position or range of positions (see, e.g., FIG. 41) to a translational displacement permitting position or range of positions (see, e.g., FIG. 40). In a translational displacement constraining position, a retention element of the depressor body 472 may be in engagement with a cooperating lock defined in the main body 20. In a translational displacement permitting position the retention element of the depressor body 472 may be disengaged with the lock of the main body 20.

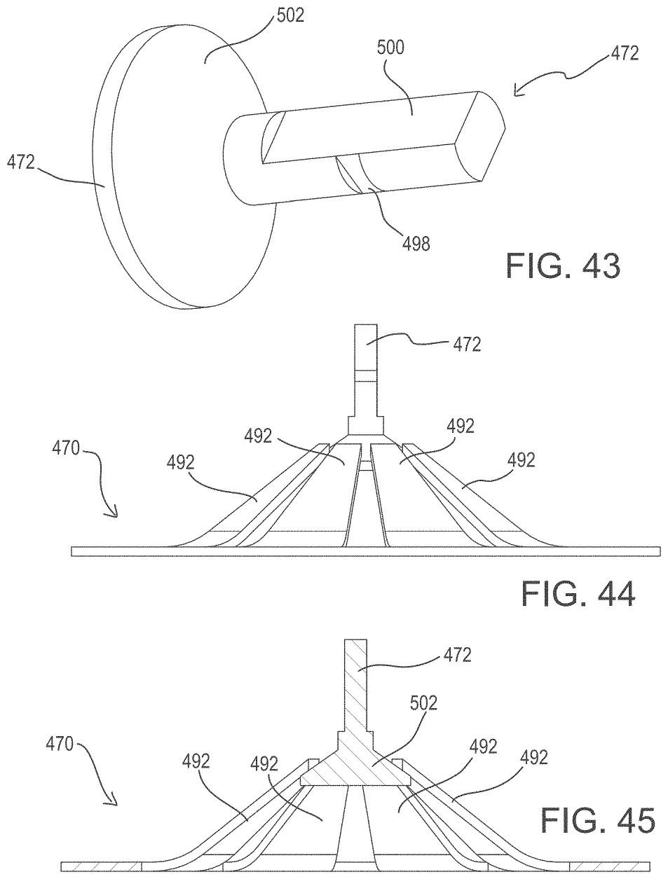

As best shown in FIG. 43, the exemplary depressor body 472 includes a stem 500 which extends through the aperture 496 in the main body 20 of a delivery device 10. The stem 500 may include a set of notches 498 or other recess(es) which each may serve as a retention element. The cross-sectional shape of the stem 500 may not be circular or a regular polygon. Thus, one of the width and length dimension of the cross-sectional shape of the stem 500 may be shorter than the other. In the example embodiment, the cross-sectional shape of the stem is an obround shape. Other cross-sectional shapes may be used in alternative examples. The notches 498 may be disposed so as to be recessed into the widest portion of the stem 500. The aperture 496 (see, e.g., FIG. 39) may have a shape which corresponds, though may be slightly larger than, the shape of the stem 500 cross-section. The notches 498 may be recessed to a depth such that when level with the wall of the main body 20 in which the aperture 496 is formed, the depressor body 472 may be rotated within the aperture 496.

The depressor body 472 is shown in a translational displacement constraining position in FIG. 41. In such a position, the rotational orientation of the depressor body 472 may be such that the notches 498 may overhang a portion of the main body 20 in which the aperture 496 is defined. As a result, the main body 20 may present a mechanical interference to translational displacement of the depressor body 472. Thus, the region of the main body 20 adjacent the aperture 496 may act as a lock for the depressor body 472. As shown in FIG. 42, when the depressor body 472 is rotated to a translational displacement permitting position, the rotational orientation of the depressor body 472 may be such that it may translationally displace within the correspondingly shaped aperture 496 of the main body.

Referring now also to FIG. 44 and FIG. 45, the depressor body 472 may include an enlarged portion 502. The reservoir interface member 474 may form a proximal region of the enlarged portion 502. When a delivery device 10 is assembled, the bias projections 492 of the bias member 470 may press upon the enlarged portion 502 capturing or coupling the enlarged portion within the bias projections 492. Additionally, the bias member 470 may be substantially constrained from displacing as a whole since the bias projections may be fed through passages 494 in the main body 20. With the bias member 470 constrained in place, lifting of the depressor body 472 may cause the bias projections 492 to deflect and become stressed. Once the depressor body 472 has been lifted such that the notches 498 are even with the portion of the main body 20 in which the aperture 496 is defined, the depressor body 472 may be rotated to a translational displacement constraining position (see, e.g., FIG. 41). Thus, the bias member 470 may be held in a stressed state.

During actuation of an associated delivery device 10 from a storage state to a delivery state, the depressor body 472 may be rotated to a translational displacement permitting position. Once this position is reached, the depressor body 472 may be free to translationally displace and the bias member 470 may urge the depressor body 472 to translationally displace. As the bias member 470 restores to a less stressed state, the reservoir interface member 474 may be driven against the reservoir 12 to force fluid out of the reservoir 12 and into a patient. The amount of the depressor body 472 which extends out of the main body 20 may alter as the bias member 470 restores to a less stressed state. Thus, the amount of depressor body 472 extending out of the main body 20 may serve as an indicator that a delivery device 10 has been used.

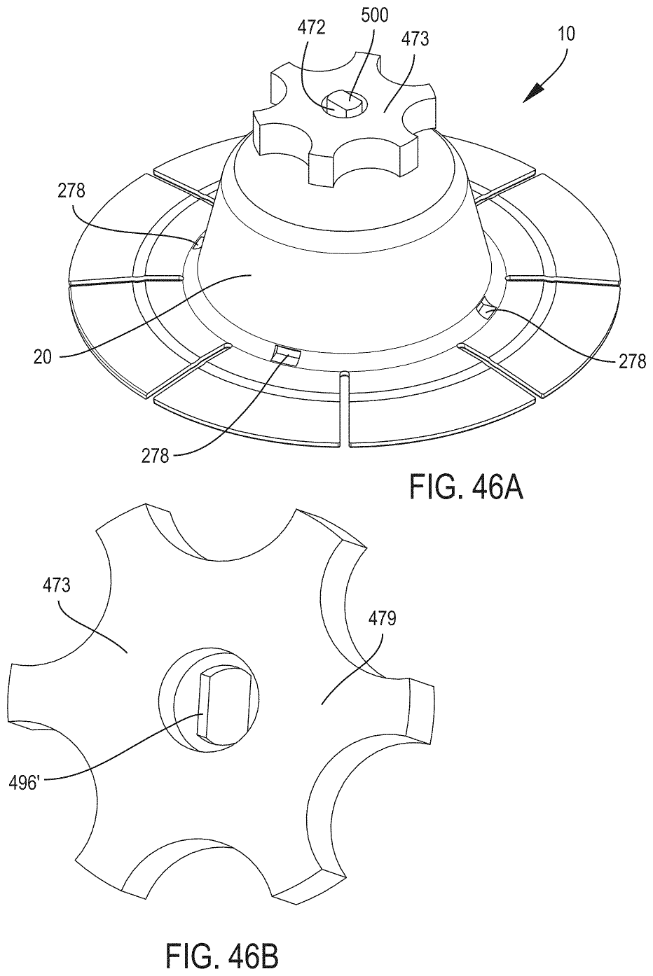

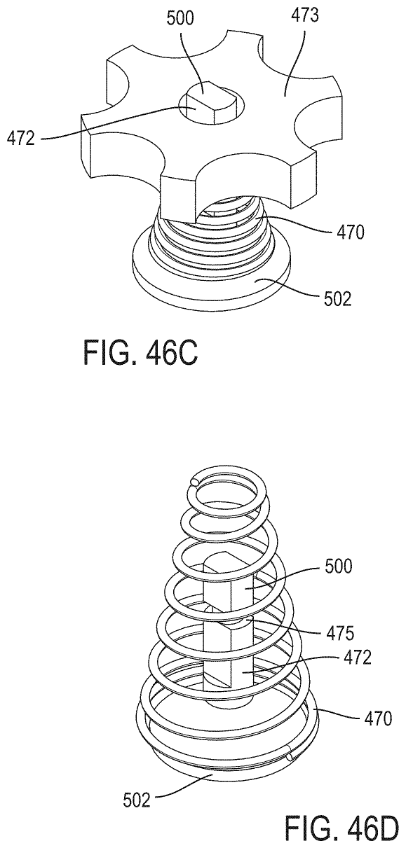

Referring now to FIGS. 46A-46D, in some examples, a bias member 470 for a delivery device 10 may be entirely internal to the delivery device 10. Additionally, the depressor body 472 may not latch or engage with a portion of the main body 20 to inhibit translation of the depressor body 472. In some examples of such embodiments, a stop member 473 may be included with the delivery device 10. The depressor body 472 may include a recess 475 (or alternatively set of notches 498, see, e.g., FIG. 43) which may engage with the stop member 473 instead of the main body 20. As best shown in FIG. 46B, the stop member 473 may include an aperture 496′ which may have a shape which corresponds, though may be slightly larger than, cross-sectional shape of the stem 500 of the depressor body 472. The recess(es) 475 may be recessed to a depth such that when level with the aperture 496′, the depressor body 472 may be rotated within the aperture 496′.

The stop member 473 may be rotated from a translational displacement constraining position to a translation displacement permitting position in which the depressor body 472 is free to displace translationally. In the translation displacement constraining position, the aperture 496′ may be positioned such that the stem 500 overhangs a portion of the body 479 of the stop member 473. As a result, the stop member 473 may present a mechanical interference to translational displacement of the depressor body 472. When the stop member 473 is rotated to a translational displacement permitting position, stem 500 may no longer overhang the body 479 of the stop member 473. In this position, the depressor body 472 may translationally displace within the correspondingly shaped aperture 496′ of the stop member 473. The stop member 473 may include ridges, knurling, bumps, grips, spokes, or other features to facilitate rotational displacement of the stop member 473 via interaction with a user's fingers.

Referring primarily to FIG. 46C and FIG. 46D, the bias member 470 may be a conical spring. The conical spring may be in a stressed (e.g. compressed state) when a delivery device 10 is in a storage state and the stop member 473 is in a translational displacement constraining position. When the stop member 473 is moved to the translational displacement permitting position, the bias member 470 may be free to drive displacement of the depressor body 472 against the reservoir 12 as described above with respect to FIGS. 44-45. As the depressor body 472 is displaced via relaxation of the bias member 470, the stem 500 of the depressor body 472 may fully pass through the aperture 496′ of the stop member 473. The stop member 473 may thus be disassociated from the rest of the delivery device 10. The depressor body 472 may also translate to a position in which the recess(es) 475 are internal to the delivery device 10. Thus, the stop member 473 may be inhibited from being recoupled to the depressor body 472. When a delivery device 10 is observed absent a stop member 473, it may provide a visual cue that the particular delivery device 10 has already been used. Thus, the stop member 473 may also serve as an indicator which conveys that a particular delivery device 10 is available for use.

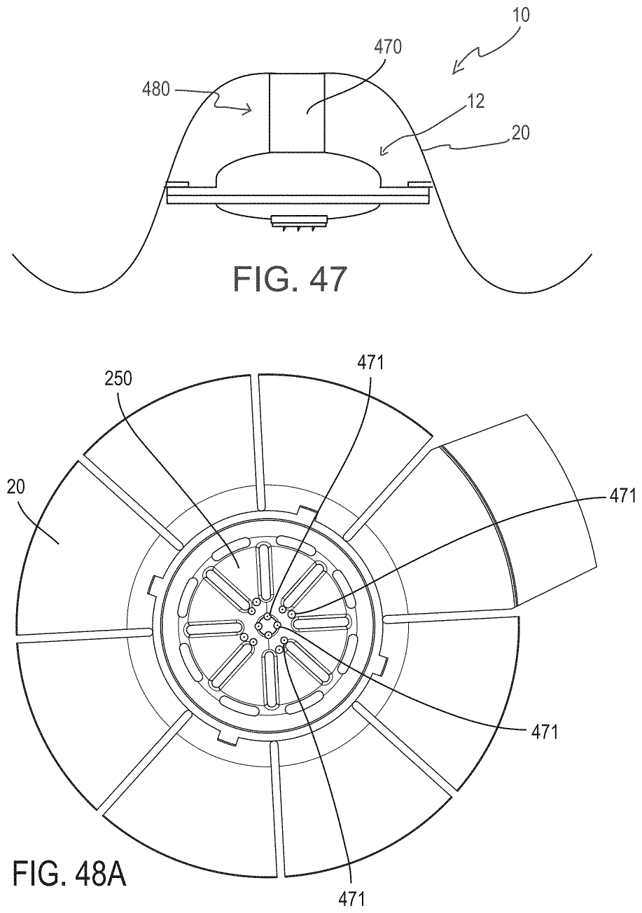

Referring now to FIG. 47, a block diagram of another exemplary delivery device 10 is depicted. As shown, the delivery device 10 may include a main body 20 and a reservoir 12. The delivery device 10 may also include one or more bias member 470. The one or more bias member 470 may form the entire dispensing assembly 480. The one or more bias member 470 may directly contact the reservoir 12 and may aid in applying pressure to the reservoir 12 in order to deliver fluid out of the reservoir 12. In certain examples, a reservoir interface member 474 (see, e.g., FIG. 36) may be included. Where included, the reservoir interface member 474 may (though need not necessarily be) be formed as a part of the at least one bias member 470 and may be integral therewith. The reservoir interface member 474 may directly contact the reservoir 12. The at least one bias member 470 may be or include a spring, compression spring, conical spring, resilient foam, air bladder, rubber body, elastomeric body, any other suitable bias member, or some combination thereof.

Still referring to FIG. 47, the bias member 470 may be in an unstressed state when the associated delivery device 10 is in a storage state. No pressure may be applied to the reservoir 12 in the storage state. In certain examples, the at least one bias member 470 (and optionally any reservoir interface member 474) may be entirely out of contact with the reservoir 12 in the storage state (e.g. by 0.05-2 mm). Alternatively, the bias member 470 may contact, but not press against the reservoir 12. When the delivery device 10 is used, the delivery device 10 may be transitioned to the delivery state as described elsewhere herein. As with various embodiments discussed herein, when transitioned to a delivery state, at least a portion of the delivery device 10 may at least partially invert. For example, at least the domed top surface 250 of the central region 28 may invert or partially invert. The distance between the reservoir 12 and the inverted top surface 250 in the delivery state may be less than the distance between the reservoir 12 and the top surface 250 in the storage state. This may in turn cause a bias to be stored in the bias member 470. The at least one bias member 470 may, in the example, be compressed when the top surface 250 is inverted. Additionally, where the at least one bias member 470 is spaced from the reservoir 12 in the storage state, the at least one bias member 470 or reservoir interface member 474 (which may be a part of the bias member 470) may be displaced into contact with the reservoir 12. The inverted top surface 250 may be sufficiently strong in the inverted state to withstand any force exerted by the at least one bias member 470. As the at least one bias member 470 restores, the at least one bias member (and/or reservoir interface member 474 if included) may press against the reservoir 12 to collapse the reservoir 12 and drive fluid into a patient. Thus without, for example, sustained manual pressure against the delivery device 10, pressure may be still applied to the reservoir 12 over a period of time sufficient to fully deliver contents of the reservoir 12 (e.g. five minutes in certain embodiments).

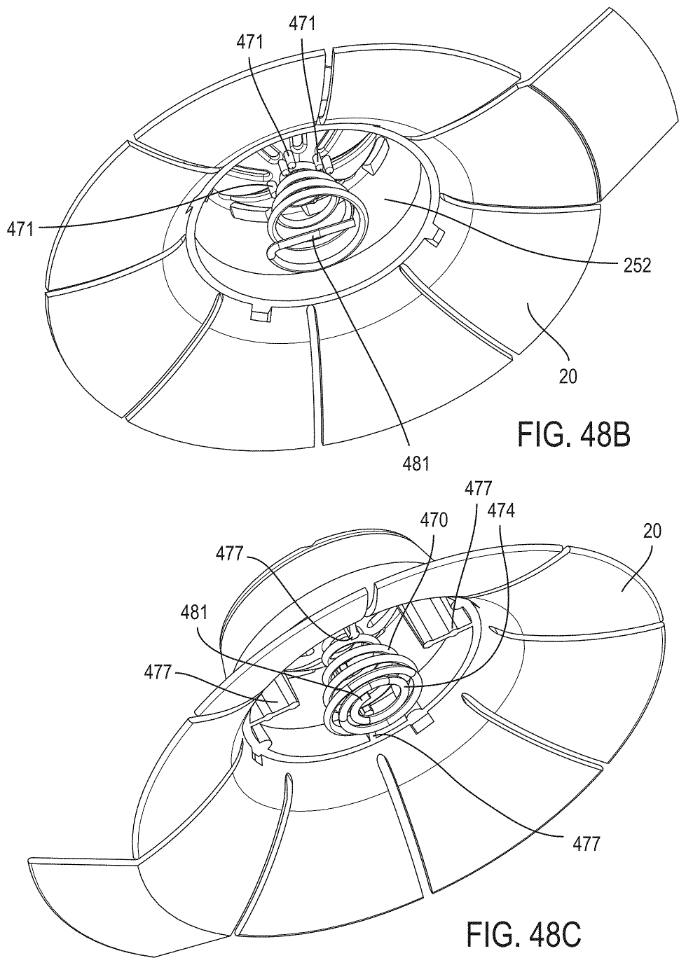

Referring now to FIGS. 48A-48B, an example embodiment of a main body 20 and a main body 20 with a bias member 470 are respectively shown. FIG. 48A depicts a bottom plan view of the main body 20. FIG. 48B depicts a perspective view of the main body 20 and bias member 470. The main body 20 is shown with the top surface inverted for illustrative purposes. As shown, the main body 20 may include a number of locating projections 471. In alternative embodiments, the locating projections 471 may be replaced with a round or annular locating wall. There may be a set of locating projections 471 which are disposed in a center region of the top surface 250. A second set of locating projections 471 may be spaced outwardly from the center region may optionally be included. In the example embodiment, the second set of locating projections 471 extend from the top surface 250. In other examples, location projections 471 may extend radially inward from the supporting structure 252 of the central region 28. An end of the bias member 470 may be centered by the locating projections 471 as the bias member 470 is placed into the delivery device 10 assembly. In certain examples, the end of the bias member 470 may be coupled into place. For example, the end of the bias member 470 adjacent the top surface 250 may be heat staked once the bias member 470 is properly positioned (see, e.g., FIG. 48C). When the delivery device 10 is fully assembled, the heat stake may retain the bias member 470 in place against the main body 20. As a result, the bias member 470 may be held out of contact with the reservoir 12 until the delivery device 10 is transitioned to the delivery state. The locating projections 471 may also aid in ensuring that the bias member 470 transitions to the stressed state in a desired manner. For example, where a compression spring is used, the second set of locating projections 471 may constrain the bias member 470 such that the bias member 470 is compressed substantially along the axis of the bias member 470.

Referring now also to FIG. 48C, in some examples, the bias member 470 may be constrained from displacement by one or more guide body 477. The one or more guide body 477 may extend from the supporting structure 252 of the central region 28 of the main body 20 toward the axis of the bias member 470. In the example shown in FIG. 48C, four guide bodies 477 are included and are spaced at even angular increments. In other embodiments, the number of guide bodies 477 may differ and/or the guide bodies 477 could be irregularly spaced. The guide bodies 477 may aid in ensuring that the bias member 470 compresses substantially along its axis and may help inhibit tilting of the bias member 470 during use of a delivery device 10.

Still referring to FIGS. 48A-C, where the bias member 470 is a compression spring, a terminal end 481 of the bias member 470 may form a reservoir interface member 474. The terminal end 481 bias member 470 may be routed in a manner which helps spread pressure more uniformly over the reservoir 12. The terminal end 481 of the bias member 470 may be routed in a direction or desired pattern. The terminal end 481 may also be disposed substantially within a plane disposed even with or adjacent an end of the bias member 470. In the example embodiment in FIG. 48B, the terminal end 481 of the coil is bent so that it extends between opposing points on the bias member 470. In the example, the terminal end 481 extends substantially diametrically across the end of the bias member 470 proximal the reservoir 12. In other embodiments, the terminal end 481 of the bias member 470 may be routed in a spiral or other pattern (see, e.g., FIG. 48C).

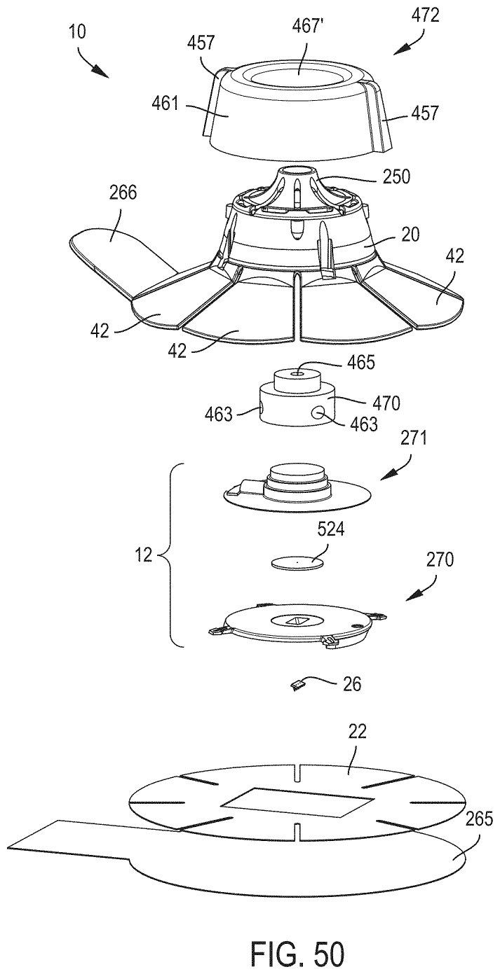

Referring now to FIGS. 49A-49B, in other examples, the bias member 470 may be block of compressible material such as rubber or elastomer. The surface of the bias member 470 adjacent the reservoir 12 may serve as the reservoir interface member 474 and may be substantially flat or planar in certain embodiments. Thus, various delivery devices 10 may include a reservoir interface member 474 which is compliant. The delivery device 10 depicted in FIGS. 49A-49B is shown in a storage state. As shown, the delivery device 10 may include a depressor body 472 which may be coupled to the top surface 250 of the main body 20. The top surface 250 of the main body 20 may have an infundibuliform or trumpet shape when in the storage state in certain examples. Such top surfaces 250 may be included in various other embodiments described herein. As shown, the depressor body 472 includes a post 469. The post 469 may extend through and be coupled to the top surface 250 (e.g. via a heat stake, adhesive, a dogged central aperture 459, etc.). The depressor body 472 may further include a dish body 467 coupled to the post 469. The dish body 467 may be disposed above the top surface 250 of the main body 20. The dish body 467 may provide an ergonomic location for a user to press against when transitioning the delivery device 10 to the delivery state. When the delivery device 10 is transitioned to the delivery state, top surface 250 may substantially invert and the bias member 470 may be compressed against the reservoir 12. This may urge fluid to be dispensed from the reservoir 12. In some embodiments, the bias member 470 may be coupled to an end of the post 469 opposite the dish body 467. For example, the bias member 470 may include a receiving recess 465 (see, e.g., FIG. 50) into which the end of the post 469 may be mated. Though a dish body 467 in the form of a concave dish is depicted, a dish like body need not be included in all embodiments. For example, the dish body 467 may be replaced by a relatively planar body or plate in certain embodiments.

Referring now to FIG. 50, an exploded view of a delivery device 10 similar to that illustrated in FIG. 49B is depicted. As shown, some delivery devices 10 may include a bias member 470 which changes in width along its height dimension and is constructed of an elastomeric material such as a silicone material. For example, the bias member 470 may be tiered. In the example shown, the bias member 470 includes two tiers. Additionally, the bias member 470 may include one or more hollow region. In the example embodiment, the bias member 470 includes a plurality of passages 463 which extend through the bias member 470 to form hollow regions. The passages 463 may extend through at least one of the tiers and in the example embodiment, both are disposed in the first or base tier of the bias member 470. The passages 463 may be evenly spaced about the bias member 470 and are disposed such that the bias member 470 has a plane of symmetry in the example shown. As noted above, when the top surface 250 of the main body 20 is transitioned from its storage state position to the delivery state position, the bias member 470 may become compressed. Fluid may be driven out of the reservoir 12 as the bias member 470 restores to a less compressed state. The passages 463 may make the initial application of force by the bias member 470 against the reservoir 12 more gentle and less abrupt. This may make reservoirs 12 more robust during use while still ensuring reservoirs 12 are substantially emptied during delivery.

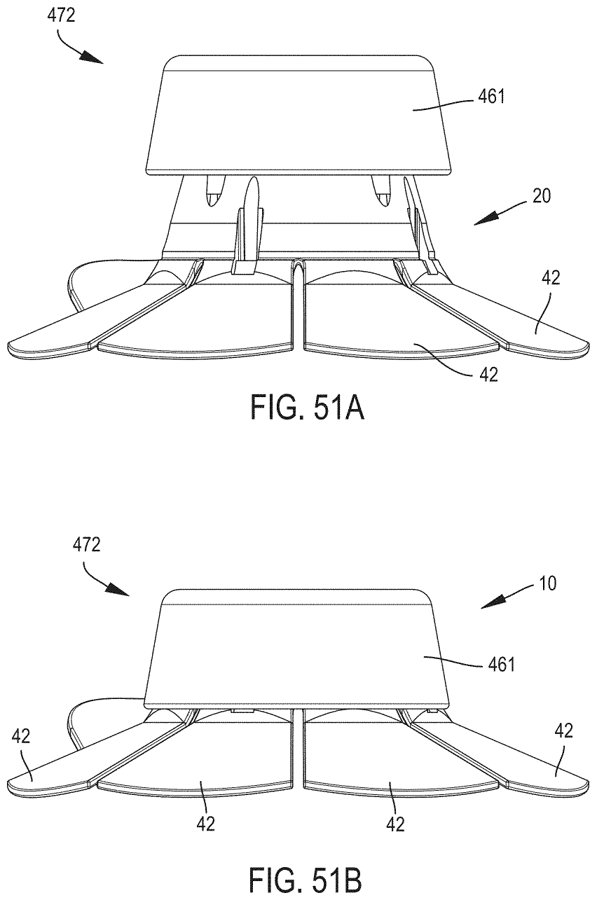

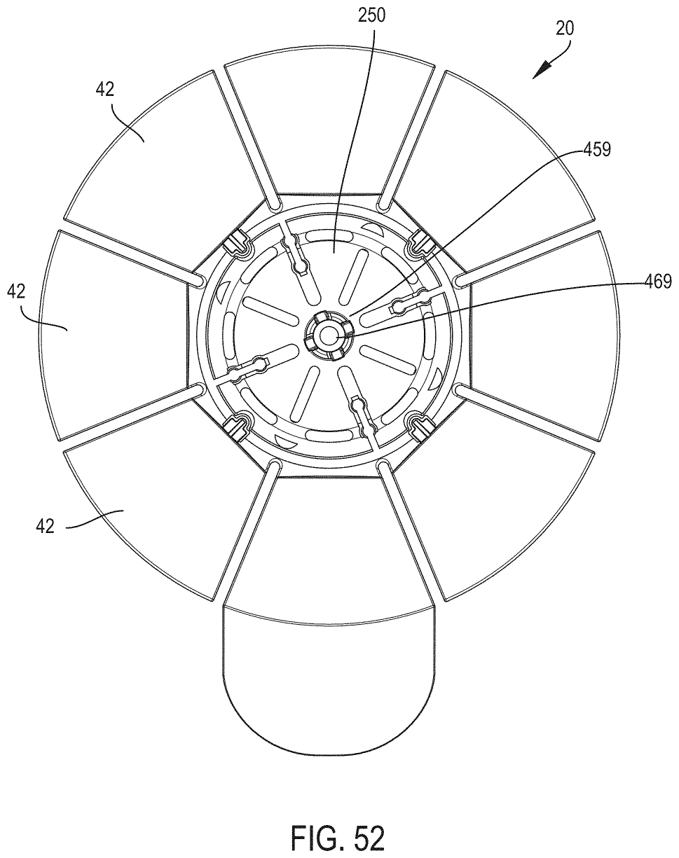

Referring now also to FIGS. 51A-52, various views of a main body 20 and a depressor body 472 are shown. The depressor body 472 of the delivery device 10 may include a dish region 467′ from which a skirt 461 extends. The skirt 461 may include a set of ears 457 extending outwardly therefrom such as those shown in FIG. 50. Any ears 457 may be spaced at regular angular intervals. The dish region 467 may be shaped similar to dish bodies 467 described elsewhere herein. The skirt 461 may be sized to nest over the supporting structure 252 of the main body 20 when the delivery device 10 is transitioned from the storage state (FIG. 51A) to the delivery state (FIG. 51B). Thus, the supporting structure 252 may act as a guide which helps inhibit tilting of the depressor body 472 and assists in ensuring that the depressor body 472 displaces substantially along an axis as pressure is applied. When transitioned fully to the delivery state, the end of the skirt 461 opposite the dish region 467′ may be near the petal members 42, but sufficiently spaced from the petals members 42 so as not to restrict movement of petal members 42. As shown best in FIG. 52, the depressor body may include a post 469. The post 469 may extend through a dogged aperture 459 in the central portion of the top surface 250 of the main body 20. As the depressor body 472 is pulled in a direction away from the main body 20, the dogs of the dogged aperture 459 may pivot and bite into the post 469 inhibiting the depressor body 472 from being disassociated from the rest of the delivery device 10. When the delivery device 10 is assembled, the post 469 may project into the receiving recess 465 in the bias member 470.

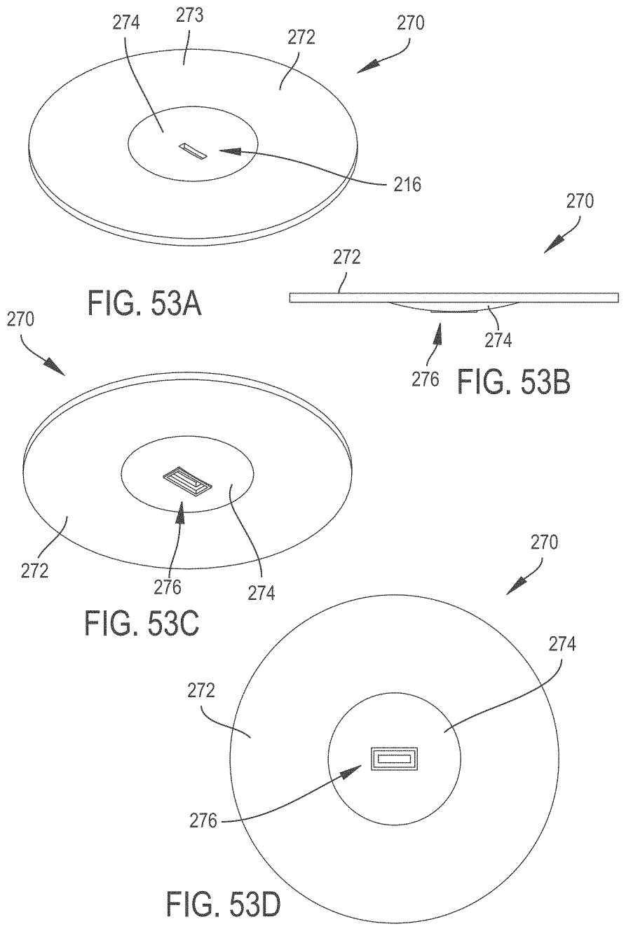

In some embodiments, as shown in FIGS. 53A-D (respectively a perspective view from the top, view from the side, perspective view from the bottom, and view from below, relative to an application surface for a delivery device 10 such as the skin surface), an example holder 270 for a sharp bearing body 26 (see, e.g., FIG. 33) including delivery sharp(s) 72 (see, e.g., FIG. 33), may be formed as an annulus or annular body 272 integral with a rounded depression 274. The rounded depression 274 may be centrally disposed. In one example, the rounded depression 274 may have the shape of a spherical segment. The annulus 272 may have an inner edge, and the rounded depression 274 may have a perimeter. The inner edge of the annulus 272 may be coincident to the perimeter of the rounded depression 274. When a delivery device 10 incorporating a holder 270 is affixed to the skin surface of a user, the rounded depression 274 extends below the plane of the annulus 272 (see FIG. 53B). Thus the rounded depression 274 may also form a bump extending proud of the proximal face of the holder 270.

The rounded depression 274 may include a pocket 276 formed therein. The pocket 276 may be formed in a proximal face of the holder 270 (e.g. in the bump). The pocket 276 may be situated at a center, and lowest (with respect to the skin surface when the delivery device 10 is affixed thereto) point of the rounded depression 274. The pocket 276 may be sized to fit and accept a sharp bearing body 26 with delivery sharp(s) 72 thereon such as, e.g., the sharp bearing body 26 including delivery sharps 72 of FIG. 33. The sharp bearing body 26 including delivery sharp(s) 72 may be mated into the pocket 276 by, e.g., injection molding or adhesive. The holder 270 may be over molded around the sharp bearing body 26 so as to couple the components together. In various embodiments, the delivery device 10 may be arranged such that pressure from above (e.g., from a finger) on the delivery device 10 may be distributed evenly over the area of the holder 270. In some embodiments, the depression 274 may act as a force concentrating protuberance from the holder 270 which serves to ensure force applied to a delivery device 10 is concentrated upon the delivery sharp(s) 72 aiding in insertion of the delivery sharp(s) 72 into the skin.

In an example embodiment, the width (e.g. diameter) of the holder 270 may be approximately 0.7 inches (e.g. 0.744 inches). The footprint area of an exemplary holder 270 may be approximately 0.45 square inches (e.g. 0.44 square inches). The holder 270 may be manufactured by any technique known to those of skill including, e.g., injection molding or thermoforming.

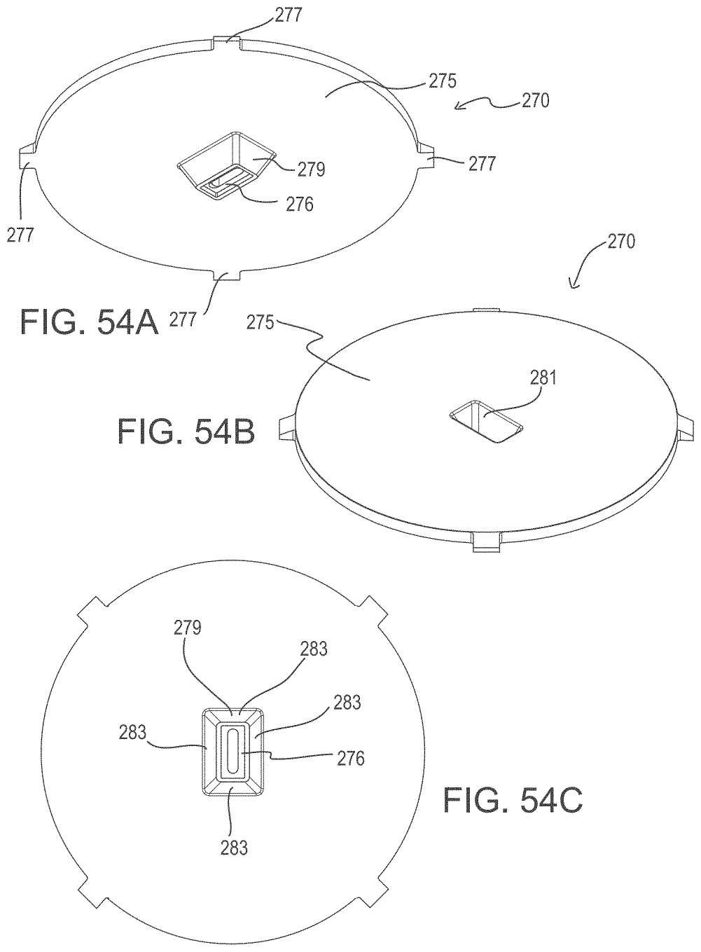

Another exemplary holder 270 is depicted in FIGS. 54A-54C. As shown, a holder 270 may include a disk body 275. The disk body 275 may be substantially flat and may include a number of peripherally disposed tab projections 277. The tab projections 277 may be symmetrically disposed about the disk body 275 and may be spaced at regular angular intervals as shown in FIGS. 54A-54C. In alternative embodiments, the tab projections 277 may be asymmetrically disposed about the base or disposed at irregular angular intervals. The tab projections 277 may engage with receiving slits 278 (see, e.g., FIG. 46A) disposed in a main body 20 of a delivery device 10. Thus, the tab projections 277 may be used to couple the holder 270 into place in a delivery device 10. Asymmetric or irregularly spaced tab projections 277 may allow for the holder 270 to be coupled to a main body 20 in a prescribed orientation which may be desirable in some examples.

Still referring primarily to FIGS. 54A-54C, a holder 270 may include at least one stage projection 279. The stage projection 279 may be included in addition or instead of the rounded depression 272 and spherical segment of the embodiment described above in relation to FIGS. 53A-53D. The stage projection 279 may provide a well 281 on the distal side of the disk body 277. The stage projection 279 may extend proud of the proximal side of the disk body 277 by a height which may, in certain examples, be at least equal to the height of a microneedle (e.g. 600 microns) of the delivery device 10. The stage projection 279 may generally extend from the disk at a perpendicular angle. The side walls 283 of the stage projection 279 may be chamfered so as to extend in a non-perpendicular direction with respect to the proximal face of the disk body 279. The stage projection 279 may include a pocket 276. The pocket 276 may be sized to fit and accept a sharp bearing body 26 with delivery sharp(s) 72 thereon as described elsewhere herein.

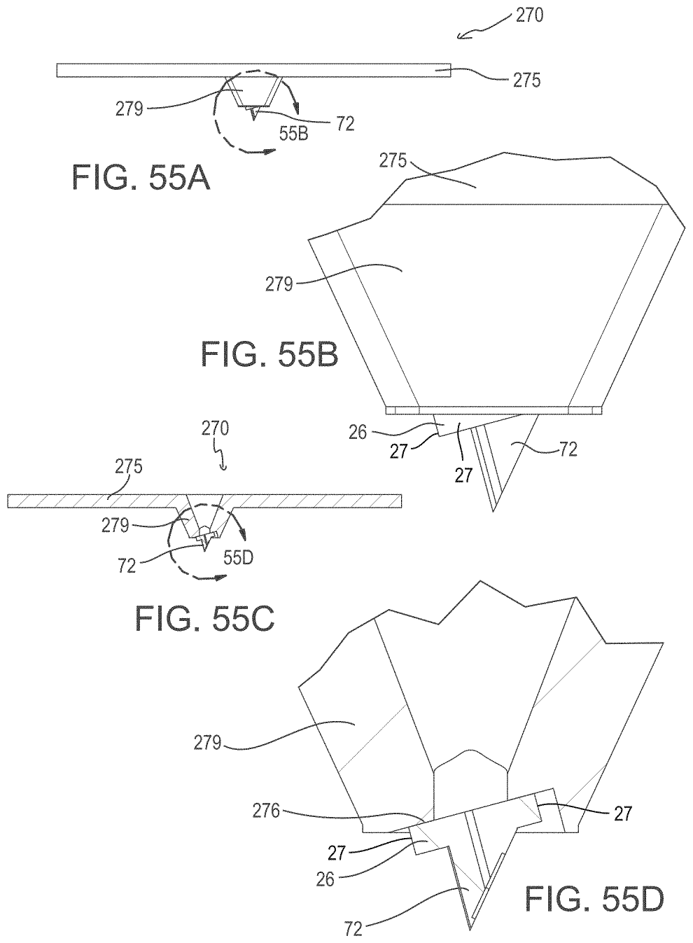

Referring now to FIGS. 55A-55D, in some embodiments, the pocket 276 of the stage projection 279 may be in a non-parallel orientation with respect to the plane of the disk body 275. As best shown in FIG. 55D, when a sharp bearing body 26 is mounted to the pocket 276, the orientation of the pocket 276 may ensure that the delivery sharp(s) 72 (e.g. microneedles) extend at a prescribed angle with respect to the disk body 275. In the example embodiment, the pocket 276 may be oriented such that the delivery sharp(s) 72 extend at a 10-20° angle (e.g. 15°) with respect to a plane perpendicular to the disk body 275. In other embodiments, the pocket 276 may be oriented such that the delivery sharp(s) 72 project at a 45° or 60° angle or some angle therebetween. Any suitable angle may be used. In alternative embodiments, the entire stage projection 279 may project at the desired angle from the disk body 275. Thus, the delivery sharp(s) 72 may extend at that angle when coupled to the pocket 276.

The sharp bearing body 26 may be coupled to any of the holders 270 described herein during a molding operation or via an adhesive. Where the sharp bearing body 26 is joined to any of the holders 270 described herein during molding, some material may be molded up the sidewalls 27 of the sharp bearing body 26 and over onto the face of the sharp bearing body 26 from which the delivery sharp(s) 72 project to capture the sharp bearing body 26. In alternative embodiments, the sidewalls 27 of the sharp bearing body 26 may be chamfered or at an angle which is not perpendicular to the face of the sharp bearing body 26 from which the delivery sharp(s) 72 extend. The footprint or cross-section of the sharp bearing body 26 may increase in area as distance from the sharp bearing face of the sharp bearing body 26 increases. Where the delivery sharp(s) 72 are silicon, a number of sets of delivery sharp(s) 72 may typically be formed on a large wafer and sharp bearing bodies 26 including the desired number of delivery sharp(s) 72 may be diced out of the wafer. To form the chamfered sidewalls 27, the dicing saw may have angled faces such that dicing process creates the desired chamfer or angle on the sidewalls 27. In certain embodiments, sidewalls 27 which are between 30-60° (e.g. 45°) may be used. Where chamfered sidewalls 27 are present, material may be molded up only a portion of the sidewall 27 to couple the sharp bearing body 26 to a holder 270. This may allow for a sharp bearing body 26 to be captured in a holder 270 (or any other molded component, e.g., a part of a delivery implement or an adapter for a syringe or other delivery implement which couples to that delivery implement via a luer lock or the like) without material being molded over onto the sharp bearing face of the sharp bearing body 26 (though this could optionally be done). Thus no molded material may act as a stand-off on the sharp bearing face blocking the full height of any delivery sharp(s) 72 from penetrating into the skin.

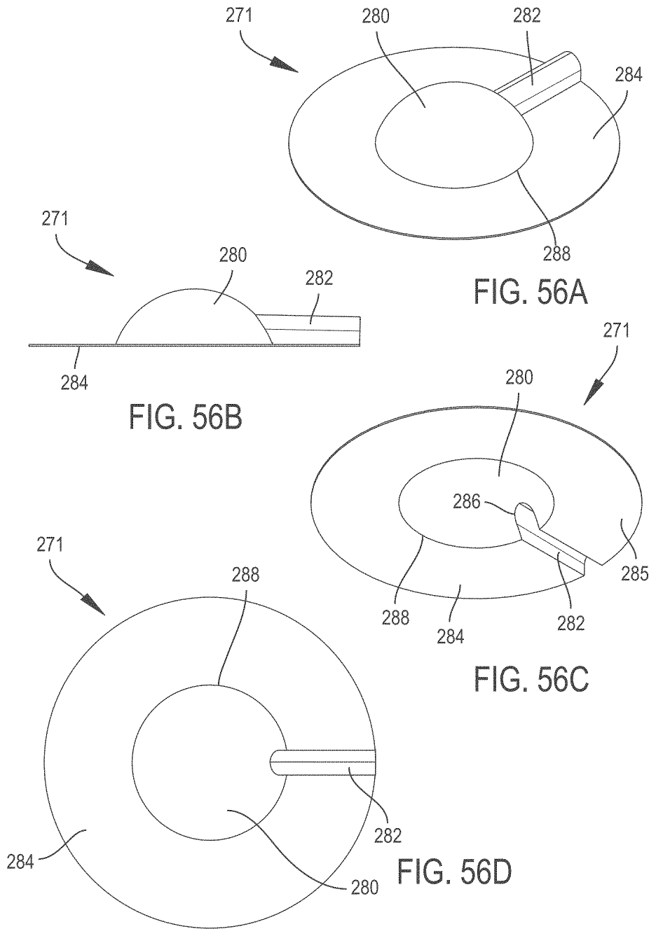

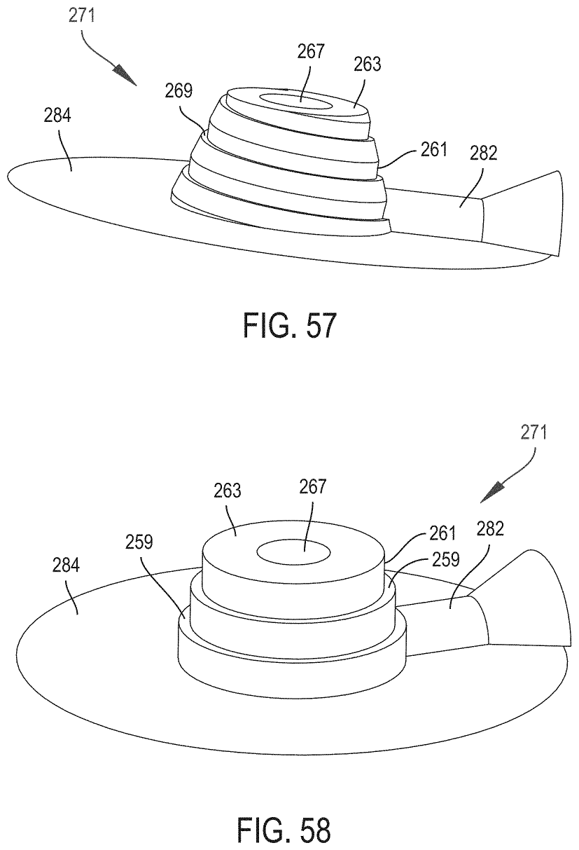

In some embodiments, as shown in FIGS. 56A-56D (respectively a perspective view looking down from above, view from the side, perspective view looking up from below, and view from the top relative to an application surface for a delivery device 10 such as e.g., the skin surface), a reservoir portion 271 is depicted. A reservoir portion 271 may be shaped to incorporate, as an integrated structure, a dome shaped portion 280, a tunnel or side channel 282, and a flange or annular portion 284. That is, these features may be included in a single monolithic piece of material. In some embodiments the dome shaped portion 280 may be shaped approximately as a hemisphere or other spherical segment though any other suitable shape is possible. In examples where the reservoir portion 271 includes a rounded shape which forms a cavity (e.g. the dome shaped portion 280), there may be a plateau or flat surface included at the portion of the rounded shape most distal the flange 284. The flat surface may be generally parallel to the flange 284. In some examples, a central depression 267 (see, e.g. FIG. 57) may also be included in the flat surface. The tunnel 282 may be shaped in some examples as a half-pipe or half cylinder that may be formed from the annular portion 284. Any suitable cross-sectional shape may be used in alternative embodiments. The side channel or tunnel 282 may communicate with the dome shaped portion 280 via an arch 286 such that the combination of dome shaped portion 280 and tunnel 282 form a structure shaped approximately as an igloo. In some embodiments, the end of the tunnel 282 opposite the dome shaped portion 280 may flare or taper outwardly to increase ease of filling. The annular portion 284 may have an inner edge that is coincident with a base perimeter 288 of the dome shaped portion 280. The reservoir portion 271 may be manufactured by, e.g., thermoforming a flat sheet of material (e.g., plastic or layers of various plastic or other material). Where a multilayer sheet is used, the sheet may include a drug or agent compatible layer, barrier layer, tie layer, etc. In some embodiments vacuum forming may be used to manufacture the reservoir portion 271. Other known techniques such as injection molding could be used. The reservoir portion 271 may be formed of a polycarbonate material or other suitable materials and may be coated with a Cyclic Olefin Polymer (COP) or any other suitable coating material. The dome shaped portion 280 may be collapsible when pressure is applied thereto.

Referring now to FIGS. 57, a perspective view of an exemplary reservoir portion 271 is shown. In certain examples, reservoir portions 271 may include at least one cavity with one or more built in collapse facilitator. The collapse facilitator may encourage the cavity to collapse in a prescribed manner and may lower the force needed to collapse the cavity. The collapse facilitator may also aid in ensuring that the cavity collapses such that any dead volume is minimized. Likewise, inclusion of a collapse facilitator may help to mitigate potential for fluid contained in a reservoir 12 to become trapped or pocketed in a region of the reservoir 12 that becomes blocked out of communication with an outlet during collapse of the cavity. Other reservoirs 12 described herein may include at least one collapse facilitator.

A collapse facilitator may be a pleated, bellows shaped, accordioned, creased, ruffled, stepped, or concertina shaped wall 261 which extends upward from the flange 284. The wall 261 may extend proud of the flange 284 and may taper (e.g. continuously or in stepwise manner) as distance from the flange 284 increases. A top wall 263 may span across the portion of the wall 261 most distal the flange 284. Thus, the wall 261 and top wall 263 may together form a cavity in the reservoir portion 271. The top wall 263 may be generally planar and extend parallel to the flange 284 in certain examples. The top wall 263 may in certain examples include a central depression 267. The central depression 267 may serve to aid in locating a reservoir interface member 474 (see, e.g., FIG. 36) or a portion of a bias member 470, 481 (see, e.g., FIG. 37 and FIG. 48B respectively). Flat top walls 263 and/or central depressions 267 may be included in other reservoirs 12 described herein. The cavity formed by the wall 261 and top wall 263 may have a generally round, circular, elliptical, oval, obround, or polygonal cross-section.

Though any pleating, bellows, accordion, crease, or ruffling pattern may be used, in certain embodiments, the wall 261 may include at least one pleat 269 in a spiral pattern. The at least one spiral pleat 269 may extend from a point adjacent the flange 284 and end at a point adjacent the top wall 263. Where the wall 261 tapers as distance from the flange 284 increases, any spiral pleats 269 may have a conical type spiral to accommodate the taper. Any spiraling pleat(s) 269 may have a pitch which causes each pleat(s) 269 to wrap around the wall 261 a plurality of times. In the example embodiment shown in FIG. 57, the spiral pleat 269 wraps around the wall 261 about three times. Such a pleat 269 may aid in assisting collapse of the cavity while fluid is urged out of a reservoir 12 during operation of a delivery device 10. Thus minimal force may be needed to deform and deplete such a reservoir 12 during use. Additionally, such a pleat 269 may assist in ensuring that little dead volume remains in the reservoir 12 after a delivery has completed. Use of a flat top wall 263 may also assist in collapse of the cavity.

Referring now to FIG. 58, the wall 261 may be stepped and include at least one step region 259. The cross-sectional area of the cavity may change at each step region 259. In the example, the cross-sectional area of the cavity is largest adjacent the flange 284 and decreases in a stepwise manner as distance from the flange 284 increases. The wall 261 includes two step regions 259 in the example shown in FIG. 58, though any suitable number may be included in alternative embodiments. As in the examples discussed above, the stepped wall 261 may aid in lowering force needed to collapse the cavity and help to direct the collapse in a prescribed manner. One example delivery device 10 embodiment including a reservoir 12 with such a stepped wall 261 is depicted in FIG. 49B. An example reservoir 12 including a stepped wall 261 is depicted in FIG. 78.

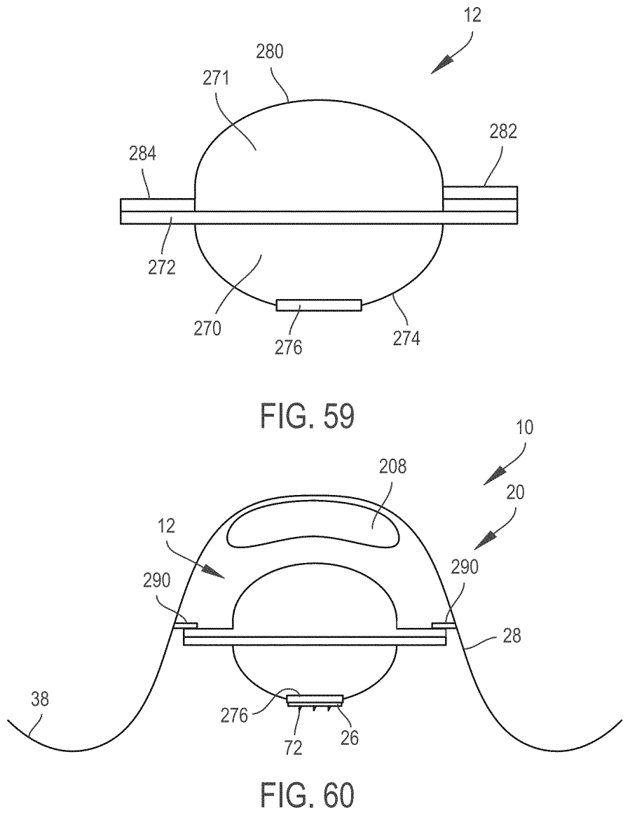

In an embodiment, the reservoir 12 may be formed by attaching the reservoir portion 271 to the holder 270, as shown in FIG. 59. The reservoir portion 271 may be positioned above the holder 270 in the example shown, and the lower surface area 285 of the annular portion 284 may be affixed to the upper surface area 273 of the annulus 272 or disk body 275. In an embodiment, the reservoir portion 271 may be attached to the holder 270 by ultrasonic welding, although any form of welding or any other coupling technique known to those of skill could be used. For example, the reservoir portion 271 and the holder 270 may be sealed together with a double sided adhesive. Other suitable techniques for sealing the reservoir portion 271 and the holder 270 together include, by way of non-limiting examples, using an ultraviolet curable adhesive, heat staking, and laser welding.

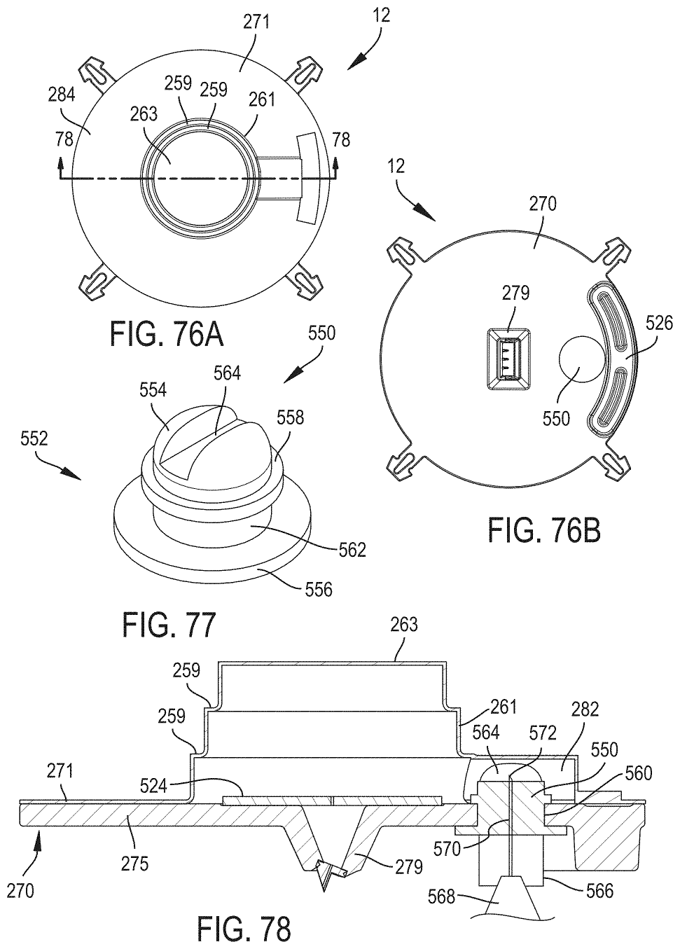

A medical agent, such as, e.g., a vaccine, may be inserted into the reservoir 12 via the side channel 282, after which the side channel 282 may be sealed closed by any known technique such as, e.g., sonic welding, heat staking, or any other suitable technique described herein. In alternative embodiments, the reservoir 12 may include a septum 550 (see, e.g., FIGS. 76A-78) through which agent may be transferred into the reservoir 12. In such examples, the side channel 282 may be omitted and the periphery of the flange 284 of the reservoir portion 217 may be completely sealed to the upper surface 273.

The sharp bearing body 26 (see, e.g., FIG. 33) including the delivery sharp(s) 72 (see, e.g., FIG. 33) may be inserted into the pocket 276 and fixed therein by any suitable technique such as, e.g., welding, prior to insertion of the medical agent (e.g., vaccine) into the reservoir 12. Alternatively, and as mentioned above, the holder 270 may be formed around the sharp bearing body 26. As mentioned elsewhere herein, the delivery sharp(s) 72 may be one or more microneedle in various examples.

In one example embodiment, the reservoir 12 may hold approximately two microliters of vaccine or other medical agent. After a medical agent (e.g., a vaccine) has been inserted, the reservoir 12 may be placed in cold chain storage separately and subsequently installed in the delivery device 10 shortly before use. This may serve to help maximize the yield of vaccine doses per unit volume in cold chain storage. The reservoir 12 may be inserted into the delivery device 10 with a packet 208 and/or container 350 or foam adhesive (such as the packet or foam adhesive material described above with reference to FIGS. 10A-11). The packet 208 and/or container 350 or foam adhesive may be disposed between the reservoir 12 and the underside of the top surface 250 of the delivery device 10 when the delivery device 10 is fully assembled. Alternatively and as discussed above, a dispensing assembly 480 (see, e.g., FIG. 36) or at least one bias member 470 (see, e.g., FIG. 47) may be disposed between the underside of the top surface 250 and the reservoir 12 once the reservoir 12 is installed.

With reference to FIG. 60, a reservoir 12 (e.g., a reservoir 12 such as that described above with reference to FIG. 59) may be affixed to the inside of a delivery device 10. Though a representational example delivery device 10 is depicted, a reservoir 12 may be similarly installed in a main body 20 of any delivery device 10 embodiments described herein. The reservoir 12 may contain a medical agent (e.g., a vaccine) prior to being assembled into the delivery device 10. The reservoir 12 may be removed from cold storage prior to being attached to the inside of the main body 20 of a delivery device 10.

Still referring to FIG. 60, in one embodiment, a ridge 290 may be formed in an inside surface of the central region 28 of the delivery device 10 such that the ridge 290 may serve as a seating structure upon or against which a section or region of the reservoir 12 may be positioned or coupled. In one example, the annulus 272 or the holder 270 may be adhered to the ridge 290 with an adhesive. It would be understood by one of skill that any suitable coupling technique could be used. In other embodiments, the distal face of the reservoir portion 271 may attach to the proximal face of the ridge 290. A distal face of a reservoir portion 271 may for example be coupled to the ridge 290 of the main body 20 of the delivery device 10 shown in FIG. 28. Tab projections 277 (see, e.g., FIG. 54A) which couple into receiving slits 278 (see, e.g., FIG. 46A) defined in the main body 20 may alternatively or additionally be used. In place of ridges 290, main bodies 20 may include retention tabs 580 and stop surfaces 582 (see, e.g., FIGS. 79-80B) which may couple the reservoir 12 in place within the delivery device 10.

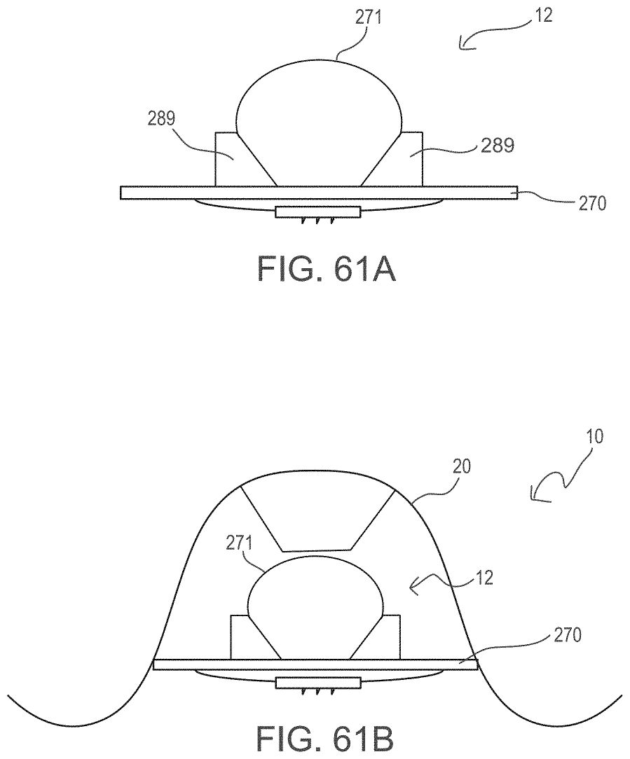

In certain embodiments and referring now to FIGS. 61A-61B, the shape of the reservoir portion 271 may be adjusted to alter the maximum cross-sectional area of the reservoir portion 271. This may aid in achieving a desired delivery pressure. For example, in some embodiments, the reservoir portion 271 may be formed so as to have a balloon like (shown in FIG. 61A-61B), cylindrical, polygonal prism shape, etc. The height of the reservoir portion 271 may be adjusted to achieve the desired interior volume given a preselected maximum cross-sectional area. As shown, the holder 270 may include at least one buttress 289. The at least one buttress 289 may at least partially surround the reservoir portion 271. The at least one buttress 289 may aid in holding the reservoir portion 271 in a desired position within a delivery device 10. The at least one buttress 289 may also aid in directing collapse the reservoir portion 271 as delivery occurs.

As shown in FIG. 61B, the main body 20 may include a nesting projection 287. As the delivery device 10 is transitioned into a delivery state (see, e.g., FIG. 31) the nesting projection 287 may press against the reservoir portion 271. As delivery progresses, the nesting projection 287 may press the reservoir portion 271 against the at least one buttress 289. In the example embodiment, the nesting projection 287 may be disposed between the exemplary buttresses 289 and may aid in ensuring minimal dead space remains in the reservoir 12 after delivery is complete.

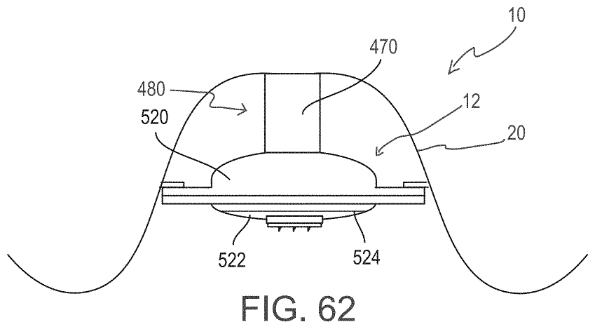

Referring now to FIG. 62, in certain embodiments, it may be desirable that delivery pressure ramp up relatively slowly when the delivery device 10 is transitioned to a delivery state. For example, it may be desirable that fluid injection begin at a relatively low pressure or at or about the lowest pressure at which injection is possible for a particular patient. The delivery pressure may be ramped up until this delivery initiation pressure is reached for a particular patient. Ramping up pressure slowly may allow for the delivery initiation pressure to be reached for a wide variety of patients using the same delivery device 10 design. Additionally, once the injection begins, it may be desirable that the delivery pressure is held at or near the delivery initiation pressure. Additionally, and as shown in FIG. 62, it may be desirable that such embodiments use at least one bias member 470 to facilitate delivery. For example, a compression spring made of a Hookean material may be used.

As shown in FIG. 62, in such embodiments, the reservoir 12 may be partitioned into a first portion 520 and a second portion 522. The first portion 520 and the second portion 522 may be in fluid communication with one another via a flow restrictor 524 (see, also FIG. 49B). The flow restrictor 524 may be disposed between a portion of the reservoir 12 proximal to the microneedles and a portion of the reservoir 12 more distal to the microneedles. The flow restrictor 524 may be an orifice plate with one or more orifice extending therethrough in certain embodiments. In some embodiments a flow restrictor 524 with a 15-25 micron orifice may be included. In other embodiments, an orifice may be up to 100 microns in diameter (e.g. 70-80 microns or 75 microns). In some embodiments, the orifice may have diameter greater than 100 microns. The orifice size may be selected based on considerations such as the viscosity and/or surface tension of the agent(s) filled into the reservoir, the desired speed of injection and how quickly it is desired to ramp up injection pressure. An orifice plate may be an injection molded component though could be formed in any other suitable manner.

Still referring to FIG. 62, the first portion 520 of the reservoir 12 may include a majority of the reservoir 12. The second portion 522 of the reservoir 12 may be disposed proximal to the delivery sharp(s) 72 relative to the first portion 520. Thus, the flow restrictor 524 may separate a large first portion 520 from a smaller second portion 522 which is most proximal the delivery sharp(s) 72. The first portion 520 may have a volume substantially equal to the fill volume of the reservoir 12 in certain examples. The flow restrictor 524 may be disposed upstream of at least the pocket 276 (see, e.g., FIGS. 53A-54C) into which a sharp bearing body 26 may be coupled. In example embodiments, the flow restrictor 524 may separate a rounded depression 274 (see, e.g., FIGS. 53A-53D) from the remainder of the reservoir 12. In such embodiments, the flow restrictor 524 may be coupled to the distal face of the annular body 272 (see, e.g. FIGS. 53A-53D) over the rounded depression 274. In other examples, the flow restrictor 524 may separate the well 281 (see, e.g., FIGS. 54A-54C) from the remainder of the reservoir 12. In such embodiments, the flow restrictor 524 may be coupled to the distal face of the disk body 275 (see, e.g. FIGS. 54A-54C) over the well 281.

In certain examples, the first and second portion 520, 522 of a partitioned reservoir 12 may be filled with different fluids. For example, the first portion 520 may be filled with an agent desired to be delivered (drug, vaccine, medical agent, etc.). The portion proximal the delivery sharp(s) 72 may be filled with a gas (e.g. sterile or cleanroom air from the manufacturing environment, inert gas, etc.). The orifice may be sized such that the properties of the agent (e.g. surface tension, viscosity) prevent the agent from passing to the second portion 522 without addition of pressure on the reservoir 12. Thus, despite the first and second portions 520, 522 being in fluid communication, the second portion 522 may remain unwetted by any agent filled into the reservoir 12 during manufacture until use. When the delivery device 10 is used, there may be a latency period during which fluid is forced into the second portion 522 from the first portion 520. Pressure in the second portion 522 may then ramp up until a pressure at which the patient's anatomy begins to accept the delivery. The pressure may remain relatively steady (or at least not spike considerably) once delivery begins.

When a delivery device 10 including a partitioned reservoir 12 is transitioned to a delivery state, at least one bias member 470 (e.g. a conical spring, foam body, rubber body, elastomeric body) may cause pressure to be exerted against the first portion 520 of the reservoir 12. Depending on the embodiment, the at least one bias member 470 may directly contact the reservoir 12 or pressure may be exerted through a reservoir interface member 474 (see, e.g., FIG. 36) or other components of a delivery assembly 280 (see, e.g., FIG. 36). The flow restrictor 524 may cause the pressure of fluid in the second portion 522 of the reservoir 512 to slowly ramp up to a pressure at which injection into a patient begins. Thereafter, the flow restrictor 524 may limit build-up of pressure in the second portion 522 as the injection progresses. Thus the injection will tend to occur at or near the lowest pressure at which the patient will accept the delivery. This may facilitate use of a more aggressive spring and may limit discomfort associated with the delivery. Moreover, it may allow for a single delivery device 10 design to be used on a wide range of patient populations (e.g. any patient) or with a wide variety of different agents. Additionally, this may have an effect on bleb formation resulting from the delivery. As the delivery may tend to occur relatively slowly and at a relatively low pressure, a more diffuse shallow (e.g. intradermal) injection may tend result. Adjustment of the size of any orifice in the flow restrictor 524 may alter the duration of the delivery and characteristics of the bleb.

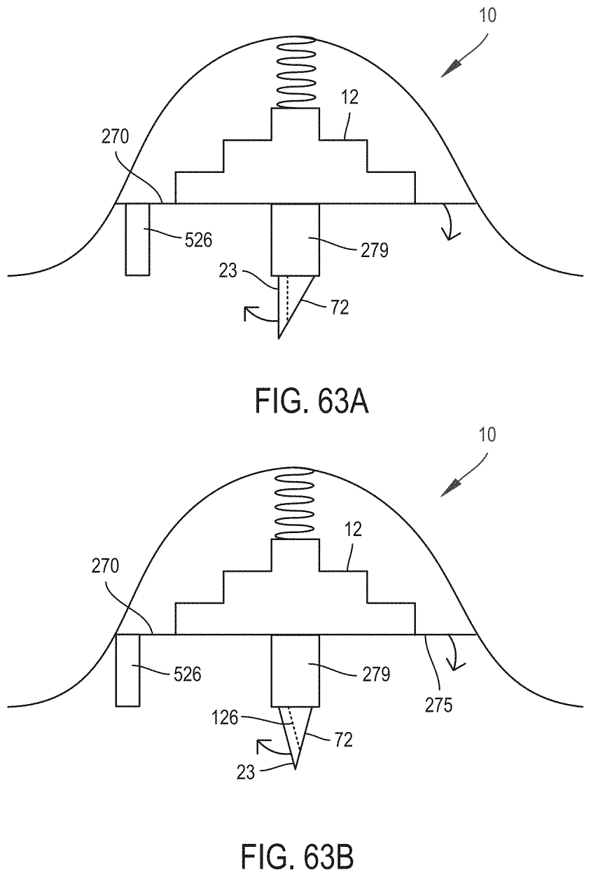

Referring now to FIGS. 63A-63B, various of the delivery devices 10 described herein may include a reservoir 12 with at least one rocker member 526. When such a delivery device 10 is applied to a user and transitioned to a delivery state, skin may be rendered taught due to spreading displacement of portions of the delivery device 10 and the at least one delivery sharp 72 of the delivery device 10 may displace into the stretched skin. Movement of the delivery sharp(s) 72 may generally be in a first direction which is substantially perpendicular to the surface of the skin and the delivery sharp(s) 72 may generally puncture downwardly into the skin. The at least one rocker member 526 may cause the reservoir 12 to tilt or rock as a consequence of the delivery device 10 being transitioned to a delivery state. The at least one rocker member 526 may cause the delivery sharp(s) 72 to displace slightly in a second direction substantially opposite the first direction when pressure is relieved from the delivery device 10. Tilting as well as displacement in the second direction may occur.

In some embodiments, portions of the delivery device 10 may also deform or adjust in response to the rocking of the reservoir 12 in order to accommodate the rocking of the reservoir 12. The tilting of the reservoir 12 may cause the delivery sharp(s) 72 to displace in a non-straight path. For example the delivery sharp(s) 72 may rotate or swing along an arcuate path during at least a portion of the transition of a delivery device 10 to the delivery state. In example embodiments, the tilting may occur automatically as a consequence of the transition of a delivery device 10 to a delivery state. No linkages or interactions with guide elements may be needed in order to achieve the tilting. Example reservoirs 12 may tilt together as a single unit due to the presence of the one or more rocker member 526. Such tilting of a reservoir 12 may lower the pressure at which injection may begin to occur and/or increase delivery flow rate in certain delivery device 10 embodiments. Additionally, the inclusion of one or more rocker member 526 may impact characteristics of bleb formation during delivery. Tilting may also help to facilitate delivery where delivery sharps 72 are initially advanced into skin at an angle substantially perpendicular to the skin.

Still referring to FIGS. 63A-63B, a rocker member 526 may be a protrusion which extends from a proximal face of a holder 270. In various examples, a rocker member 526 may be disposed at or inward of the peripheral edge of the holder 270. A rocker member 526 may have a height which is approximately the height of a stage projection 279 (see, e.g., FIGS. 54A-55D). Shorter and taller rocker members 526 are also possible. Alternatively, where a holder 270 includes a rounded depression 274 (see, e.g., FIG. 53A), the rocker member 526 may have a height which sets it approximately even with the height of a bump corresponding with the rounded depression 274. Again, shorter and taller rocker members 526 may be provided in such embodiments.

When delivery devices 10 including at least one rocker member 526 are transitioned to a delivery state, the rocker member(s) 526 may come into contact with the user and impede further displacement of the portion of the reservoir 12 including the rocker member(s) 526. The opposing side may be free of any rocker members 526 and the reservoir 12 may tilt or rock to accommodate continued displacement of the opposing side toward the user. In certain examples, the delivery sharp(s) 72 (e.g. microneedles) may tilt 3-5° (e.g. 4°) with respect to their initial orientation. In other examples, the delivery sharp(s) 72 may tilt lesser or greater amounts. Height of a rocker member 526 may alter the point at which the delivery sharp(s) 72 begin to rotate or swing during the transition of the delivery device 10 to the delivery state. Rocker members 526 even with the height of a stage 279 may, for example, tend to initiate tilting after the delivery sharp(s) 72 have punctured the skin.

In certain examples, the delivery sharp(s) 72 may be microneedles such as any of those described herein. Where the delivery sharp(s) 72 is/are microneedle(s), the rocker member(s) 526 may be disposed on a side of the reservoir 12 closest the back facing edge 23 (see, e.g., FIG. 2) of the microneedle(s). The rocker member(s) 526 may be positioned such that back facing edge 23 of the microneedle(s) is the portion of the microneedle(s) most proximal the rocker member(s) 526. As rocking of the reservoir 12 transpires, the displacement path followed by the microneedle(s) may be such that the back facing edge(s) 23 may be driven through the skin. The beveled surfaces leading to the back facing edge 23 may facilitate cutting of the skin as the microneedle(s) are displaced. Thus, the back facing edge 23 may be a cutting edge. Additionally, this may cause a face of each microneedle in which an outlet of the lumen 126 of that microneedle is disposed to be displaced away from skin contacted during the initial puncture. For example, the lumen(s) 126 of any microneedles may be displaced away from skin contacted by the sloped face(s) 21 during the initial puncture where a microneedle such as that shown in FIG. 2 is utilized. Such displacement of the microneedle(s) may aid in ensuring fluid may easily flow out of the lumen(s) 126 and into the skin as delivery occurs. The above described displacement may also create a small receiving volume in the skin into which fluid may be delivered from the lumen(s) 126. When pressure applied to the delivery device 10 to transition the delivery device 10 to the delivery state is relieved, the delivery sharp(s) 72 may displace slightly in a direction away from the patient. This may create a small receiving volume in the skin and displace the lumen 126 away from skin contacted during initial puncture. The rocker member 526 may help to encourage this.

In some examples (see, e.g., FIGS. 55A-D, FIG. 63B), delivery sharp(s) 72 may be mounted to a stage 279 having a mounting area (e.g. a pocket 256) which is non-parallel with respect to a disk body 275 of the holder 270. In such examples, the delivery sharp(s) 72 may extend from the stage 279 at a prescribed angle (e.g. 15°) with respect to a plane normal to the disk body 275. The disk body 275 and skin may be generally parallel when various example delivery devices 10 are first applied to a user. Delivery sharp(s) 72 may thus be angled with respect to a plane normal to the skin. As the reservoir 12 tilts, the delivery sharp(s) 72 may be displaced to a position in which they are closer (e.g. 3-5°) to a normal orientation with respect to the skin. Depending on the mounting angle of the delivery sharp(s) 72, the delivery sharp(s) 72 may be brought to or nearly to a normal orientation with respect to the skin as the reservoir 12 tilts. In other embodiments, the delivery sharp(s) 72 may be 10° or more (e.g. 11-12°) away from a normal orientation.

Still referring to FIGS. 63A-63B, in some examples, a reservoir 12 may include at least one marking member. The at least one marking member may contact or press against the skin as the delivery device 10 is dispensing agent to a user. In some embodiments, any rocker member(s) 526 may double as marking members though dedicated marking members may also be present in some embodiments. Marking members may also be used in embodiments which do not include a rocker member 526. When a delivery device 10 is used, one or more marking member may leave a perceptible marking on the skin. This marking may be a temporary impression or depression in the skin resulting from the marking member pressing on the skin as a delivery occurs. Alternatively or additionally, the marking member (e.g. rocker member 526) may bear a marking agent (e.g. ink) which at least partially transfers to the skin when the delivery device 10 is used. As the contact surface of the marking member may be of a known size, the perceptible marking may serve as a fiducial reference. The delivery site may, in some examples, be imaged after a delivery device 10 is removed and an image of the delivery site may be analyzed (e.g. any suitable image processing such as edge detection may be used) by a controller to identify the perceptible marking. In certain examples, it may be required that an image be taken within some preset time after a delivery device 10 is removed. The perceptible marking may aid in confirming a delivery device 10 has been used when identified in an image. Additionally, the perceptible marking could aid in determining, for example, one or more attribute related to the delivery (e.g. bleb presence, size, area, relative location of the bleb to the mark). For example, a controller may analyze an area of an image a defined distance from the marking for the one or more attribute related to the delivery or the controller may ensure that an attribute of interest conforms to an expected relationship with the marking (e.g. is within some range of distances from the marking). The mark left by a marking member or the arrangement of marking members on the delivery device 10 could be selected to leave a perceptible mark having a particular pattern. Any suitable pattern could be used. The pattern could be selected to assist in image analysis or perhaps could be selected to provide a patient friendly delivery confirmation marking (e.g. smiley face or the like).

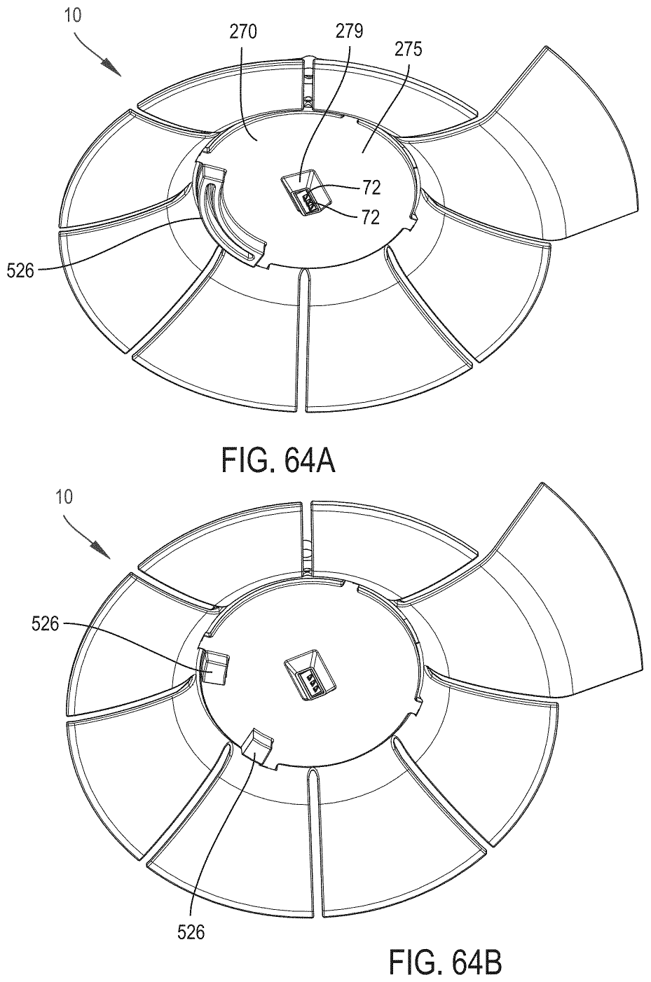

Referring now to FIGS. 64A-64B, bottom perspective views of exemplary delivery devices 10 (with adhesive members 22 hidden) including rocker members 526 are depicted. As shown, a rocker member 526 may be a protuberance from the disk body 275 which may extend along the peripheral edge of the disk body 275. The rocker member 526 in the example shown in FIG. 64A is arcuate, though rocker members 526 extending in a straight, zigzag, undulating, meandering or other path may also be used. In other examples such as that shown in FIG. 64B, multiple (two are shown, though more could be included) rocker members 526 may be included. Rocker members 526 may for example be included as a set of bumps or nubs which extend proud of the proximal face of the disk body 275. Rocker member(s) 526 may be sized and/or positioned such that rocking or tilting of the reservoir 12 occurs generally along a desired plane (e.g. a plane including or parallel to a desired displacement path taken by one or more delivery sharp 72 of the delivery device 10 as tilting occurs). The position and/or size of the rocker member(s) 526 may impact characteristics of a bleb formed during delivery for some delivery devices 10. For example, the rocker member 526 of FIG. 64A may tend to encourage a bleb to spread more in directions left and right of the center of the rocker member 526.

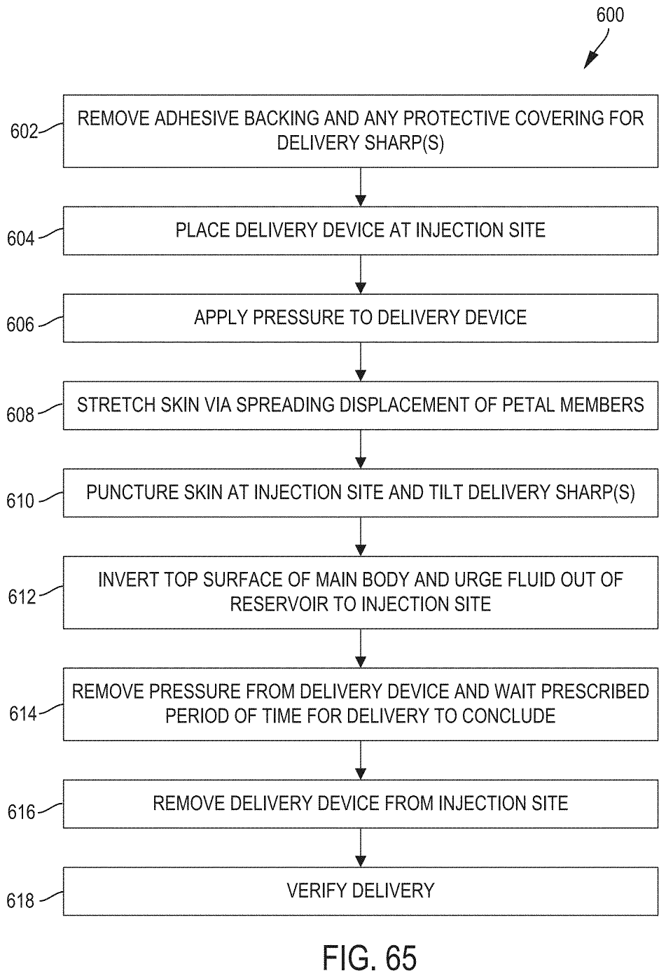

Referring now to FIG. 65, a flowchart 600 depicting a number of exemplary actions which may be executed to deliver agent from a delivery device 10 to an injection site is shown. As shown, in block 602, an adhesive backing 265 (see, e.g., FIG. 24 and FIG. 26) may be removed from a delivery device 10. Additionally, any covering (e.g. cover 409 of FIG. 26) protecting the delivery sharp(s) 72 may be removed from the delivery device 10 in block 602. In block 604, the delivery device 10 may be placed at a desired injection site.

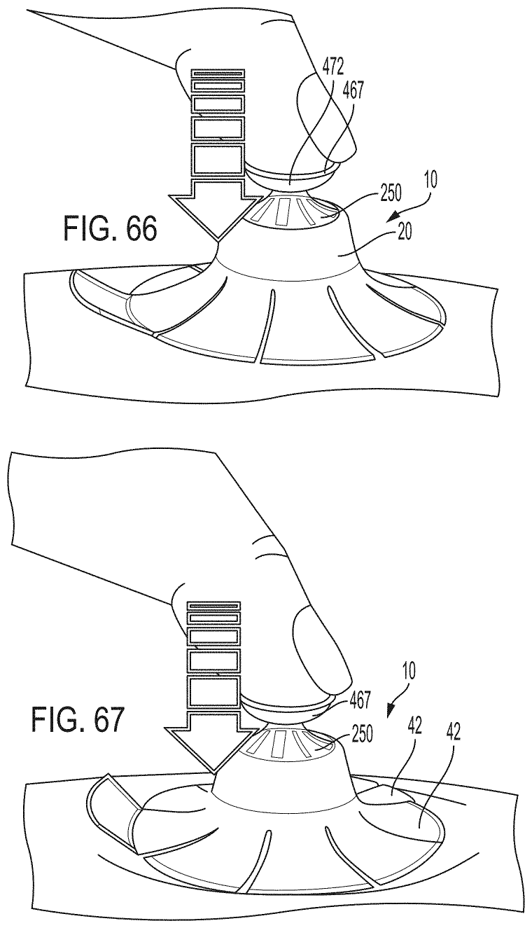

Referring now also to FIG. 66, pressure may be applied to the delivery device 10 in block 606. In some examples, pressure may be applied to the top surface 250 of a main body 20 of a delivery device 10. Alternatively, and as shown in the illustration in FIG. 66, pressure may be applied to a depressor body 472 (e.g. a dish body 467) of the delivery device 10. Pressure may be applied manually, for example, with a single finger of a user.

Referring now primarily to FIG. 65 in combination with FIG. 67, in block 608, the skin coupled to the delivery device 10 via adhesive 22 of the delivery device 10 may be stretched. At least two portions of the delivery device 10 adhered to the skin may distort from their initial state so as to spreadingly displace and stretch the skin. For example, this may occur as petal members 42 of the main body 20 distort under pressure being applied to the delivery device 10. As described elsewhere herein, when the petal members 42 distort, the reservoir 12 and delivery sharp(s) 72 coupled thereto may displace toward the skin. The delivery sharp(s) 72 may puncture the skin and may optionally tilt (e.g. due to the presence of a rocker member 526, see FIGS. 63A-64B and related description) in block 610. As shown in FIG. 67, the top surface 250 of the delivery device 10 may resist deformation at least as the petal members 42 initially begin to spreadingly displace.

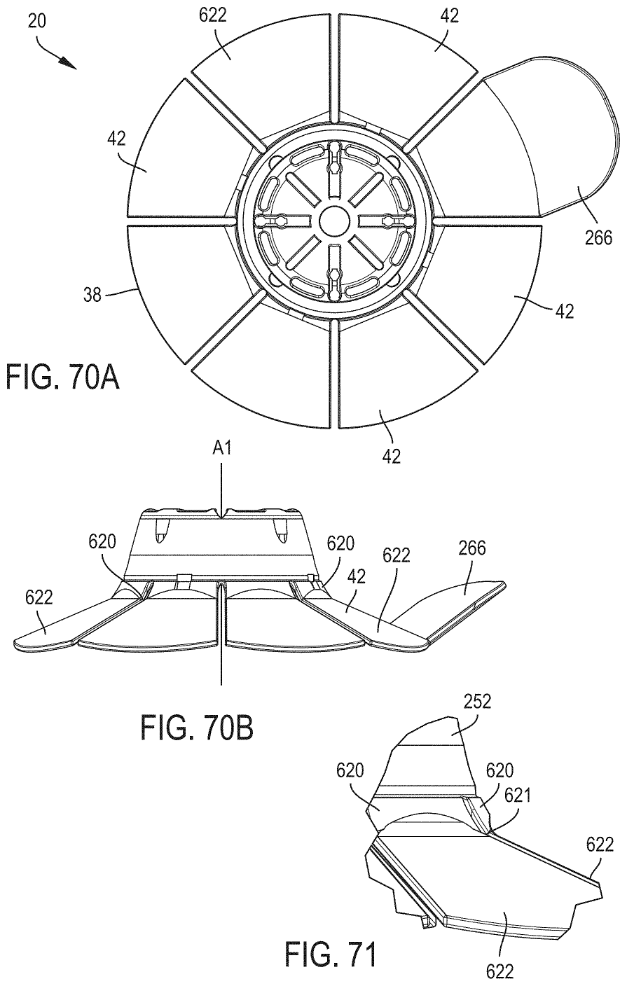

The petal members 42 may be constructed to have a stiffness selected to help ensure that this occurs. The petal members 42 may for example be substantially or primarily planar and project from the rest of the delivery device 10 at a constant angle (see, e.g., FIGS. 70A-70B). This may allow the petal members 42 to deflect relatively easily as pressure is applied to the delivery device 10. In alternative embodiments, the petal members 42 may have curvature at least in certain regions. Any curvature in the petal members 42 may be selected to ensure that at least a desired amount of distortion of the petal members 42 occurs prior to deformation of the top surface 250. In preferred embodiments, the main body 20 may be constructed such that the petal members 42 distort an amount sufficient to stretch the skin and allow the delivery sharp(s) 72 to penetrate the skin prior to the top surface 250 substantially deforming.

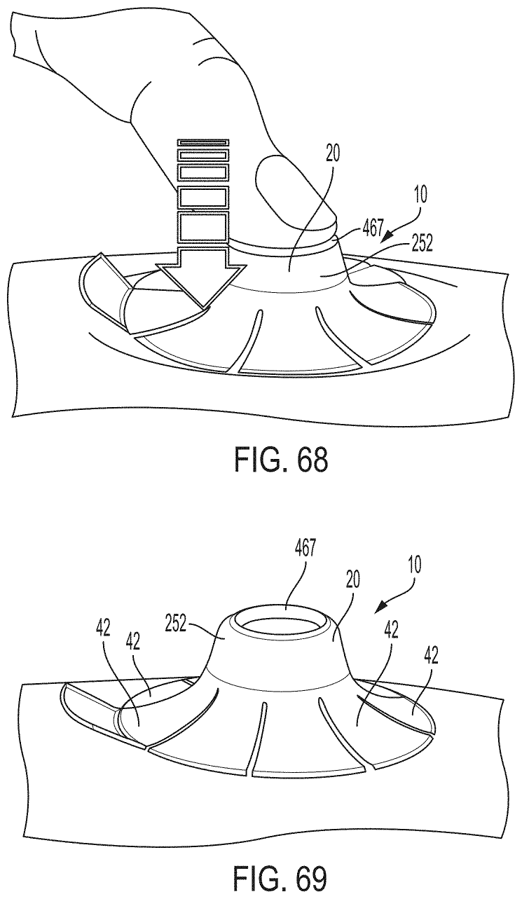

Referring now primarily to FIG. 65 in combination with FIG. 68, the top surface 250 of the main body 20 may flip from a protruding state to a depressed state or invert in block 612. Fluid may also be urged out of the reservoir 12 in block 612. As described elsewhere herein, a bias member 470 may become compressed upon flipping of the top surface 250. Fluid may be driven out of the reservoir 12 into the injection site via the delivery sharp(s) 72 as the bias member 470 restores to less distorted state. As shown in FIG. 68, in embodiments including a depressor body 472 with a dish body 467, the dish body 467 may seat against an end of the supporting structure 252 when the top surface 250 inverts. The dish body 467 may obstruct view of the top surface 250 when the top surface 250 is inverted giving a visual cue the delivery device 10 has been used (see also FIG. 69).

Referring now primarily to FIG. 65 in combination with FIG. 69, in block 614, pressure may be removed from the delivery device 10 and a predetermined period of time may be allowed to elapse. The period of time may be selected to be at least as long as and preferably greater than an expected delivery time. In certain examples, the wait time may be (1-5 minutes).

In certain examples and as shown in FIG. 69, when pressure is removed, one or more portion of the delivery device 10 may at least partially restore from its distorted state. Thus, the main body 20 may have at least one invertible region, at least one resilient region, and at least one region which elastically deforms as a delivery device 10 is transitioned to a delivery state. The at least one region which elastically deforms may be distorted from an initial state, to an intermediate state, and then elastically restore at least partially from the intermediate state during the course of the transition to the delivery state. The intermediate state may be a state during the transition in which the region is maximally distorted. The region may restore from this state back towards the initial state. The petal members 42 may, for example, at least partially restore from their distorted state. As the petal members 42 of the delivery device 10 are adhered to the skin via the adhesive 22 of the delivery device 10, the skin may be pulled away from the underlying anatomy as the petal members 42 restore. As the petal members 42 restore, the reservoir 12 may also displace slightly in a direction away from the skin (it is possible a rocker member 526 may assist this see e.g. discussion of FIGS. 63A-64B). Where adhesive 22 is present on portions of the reservoir 12 (e.g. the holder 270) skin may also be pulled away from underlying anatomy as the reservoir 12 displaces. This may relieve some pressure on the injection site which otherwise tends to compress anatomy at the injection site. This decreased compression at the injection site may allow fluid to be more readily be transferred from the delivery sharp(s) 72 into the delivery destination. Additionally, depending on the orientation of the delivery sharp(s) 72, the delivery sharp(s) 72 may tug the skin into which they have punctured upward away from underlying anatomy as the petal members 42 restore. Again, this may help to facilitate delivery as the compactedness of the anatomy at the delivery destination may be reduced. The shape of the petal members 42 and material used to construct the main body 20 may be selected to help encourage this at least partial restoration or recoil of the petal members 42 when pressure is removed.

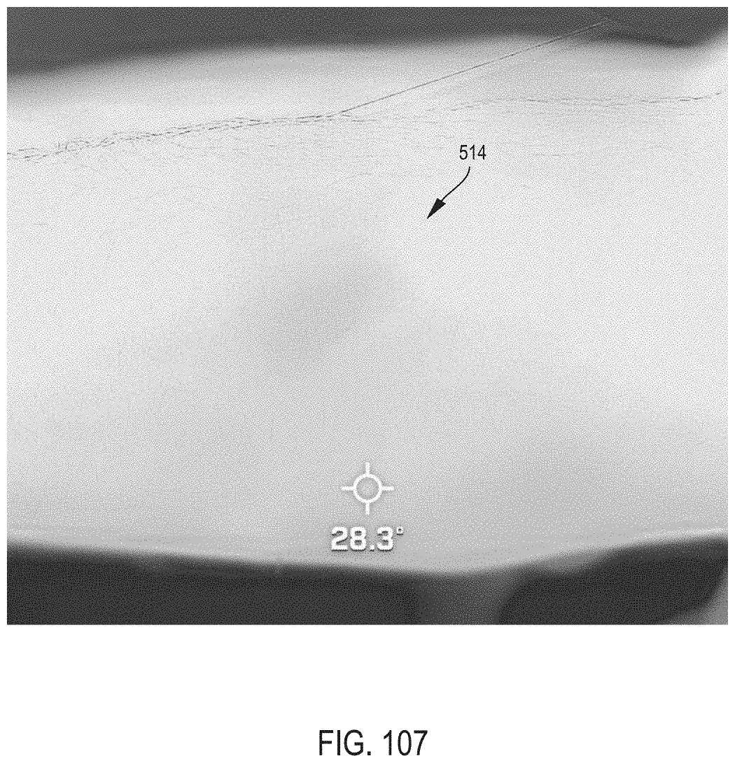

In block 616, the delivery device 10 may be removed from the injection site. In certain examples, delivery may be verified in block 618. In certain embodiments, this verification may be manual. Staff at a vaccination site or clinic may verify by eye that a bleb is present after injection and no leaks are seen. In other embodiments, an image of the injection site may be taken, analyzed, and perhaps documented in electronic records as described elsewhere herein. Image analysis may, for example, determine whether an expected marking is present at the injection site or whether characteristics of interest are present in the image (or have a desired relationship with a fiducial marking). In other examples, an image may be analyzed for the presence of a cool region in the skin (see, e.g., FIG. 107 and related description).

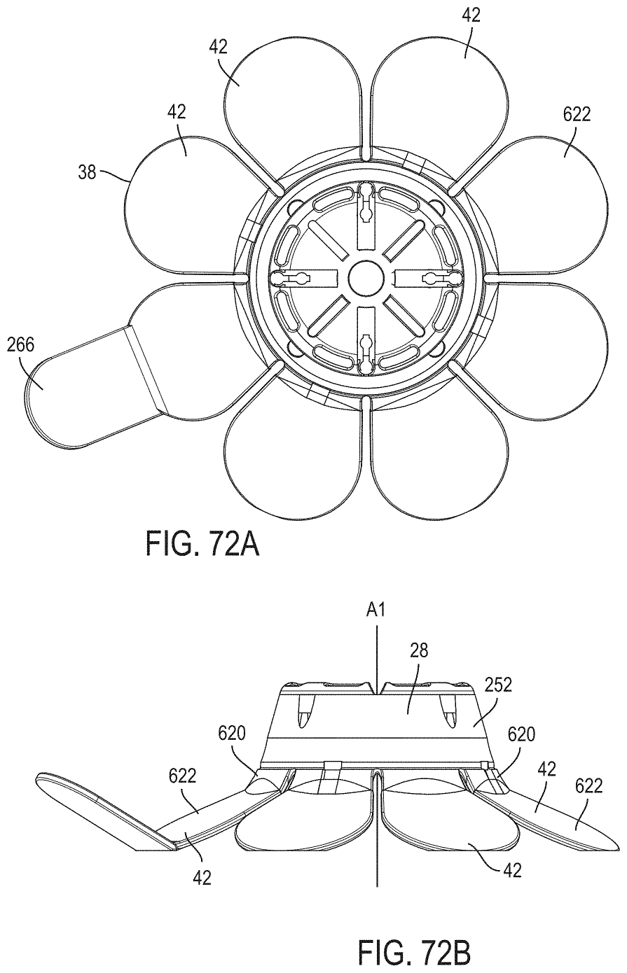

Referring now to FIGS. 70A-70B and FIGS. 72A-72B, two exemplary main bodies 20 which may be used in various delivery devices 10 described herein are respectively shown. In various embodiments where delivery devices 10 are or may be injection molded, portions of the main body 20 may be injection molded so as to be in the storage state or in the delivery state. Portions of the main body 20 may transition more easily or tend to restore into the configuration in which they were molded. Such portions may also have a tendency to stay in the configuration in which they were molded. In the examples in FIGS. 70A-70B, the top surface 250 is molded in a delivery state position. The petal members 42 are molded in a storage state position. During assembly of a delivery device 10, the various portions of the main body 20 may be brought into a storage state configuration and remain in that configuration until use. Molding the top surface 250 in the delivery state position may lower the effort needed to transition a delivery device 10 from a storage state to a delivery state. Likewise, molding the petal members 42 in their storage state positions encourages the petal members to 42 to restore toward their storage state positions (see, e.g., FIG. 69 and related description) when a user relieves pressure on the delivery device 10 during use.

As mentioned above, in certain embodiments, petal members 42 may be relatively devoid of curvature. For example, petal members 42 be substantially flat and/or extend from the rest of a main body 20 at an angle or angles thereto. This may assist in making the force required to cause deflection in the petal members 42 relatively low as pressure is applied to the delivery device 10. In turn, this may help to assist in generating spreading displacement of the petal members 42 and help ensure puncture of the skin with the delivery sharp(s) 72 prior to deformation of the top surface 250 of the main body 20.

As shown in FIGS. 70A-70B, the petal members 42 of example main bodies 20 may each include first regions 620 adjacent the supporting structure 252 and second regions 622 which form the more peripheral portions of the petal members 42. As shown, the first regions 620 may be arced roughly similar to that of the adjacent portion of the supporting structure 252. The second regions 622 may be oriented at a constant angle to the center axis A1 of the main body 20. The second regions 622 may form the majority of the petal members 42. In some examples, the petal members 42 may have a curved region or surface, while being predominantly flat. In various embodiments, a small curved transition 621 between the first and second regions 620, 622 of the petal members 42 may be included (see, e.g., FIG. 71).

A living hinge may be formed at the transition between the first and second regions 620, 622. As pressure is applied to the top surface 250 of a main body 20, the living hinge may allow the second regions 622 of the petal members 42 to displace relative to the first regions 620. The first regions 620 may distort to a lesser degree than the second regions 622 throughout the transition of the delivery device 10 to the delivery state. In some examples, the first regions 620 may resist substantial deformation and remain generally undistorted throughout the transition. Thus, the first regions 620 may behave as stops which may help to limit spreading displacement of the petal members 42 after a desired amount of spreading displacement has been achieved. Curved transitions 621 may be included to assist in encouraging the petal members 42 to at least partially restore once pressure on the delivery device 10 has been relieved. In examples including petal members 42 such as those in FIGS. 70A-70B, it may be preferred that, in the storage state, the base of any stage projection 279 be substantially even with the end of the second region 622 of the petal members 42 most proximal the central region 28.

The main body 20 may have a round, substantially circular footprint in examples such as that shown in FIG. 70A-70B. The portion of each petal member 42 at the peripheral edge 38 of the main body 20 may arc along a radius extending from the center axis A1 of the main body 20. In various examples and referring now to FIGS. 72A-72B, the width of the portions of the petal bodies 42 forming the peripheral edge 38 may decrease as distance from the central region 28 of the main body 20 increases. For instance, the portion of the petal members 42 forming the peripheral edge 38 of the main body 20 may be arced other than along a radius extending from a center point within the center axis A1 of the main body 20. For example, a radius defining the curve of the periphery of each petal member 42 may be extended from a center point within the respective petal member 42 or within the second portion 622 of the respective petal member 42. The outermost region of each of the petal members 42 may disposed more distal the central region 28 than the ends of the slits 36 most distal the central region 28. The outermost regions may decrease in width as distance from the central region 28 increases. In certain examples, the portion of each petal body 42 forming the peripheral edge 38 may taper to a point or be rounded as shown in FIGS. 72A-72B.

This may assist in removal of a delivery device 10 as it may decrease the surface area of adhesive which a user is attempting to dissociate from the skin during initial portions of the removal action. Though each petal member 42 may be described as having the same shape, it should be understood that such description may be inclusive of at least one petal member 42 differing in shape slightly to accommodate a pull tab 266 (see, e.g., FIG. 72B) which may be included to assist in removal of the delivery device 10.

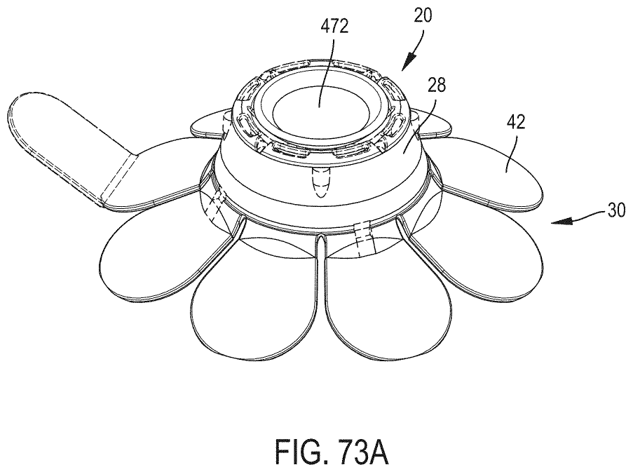

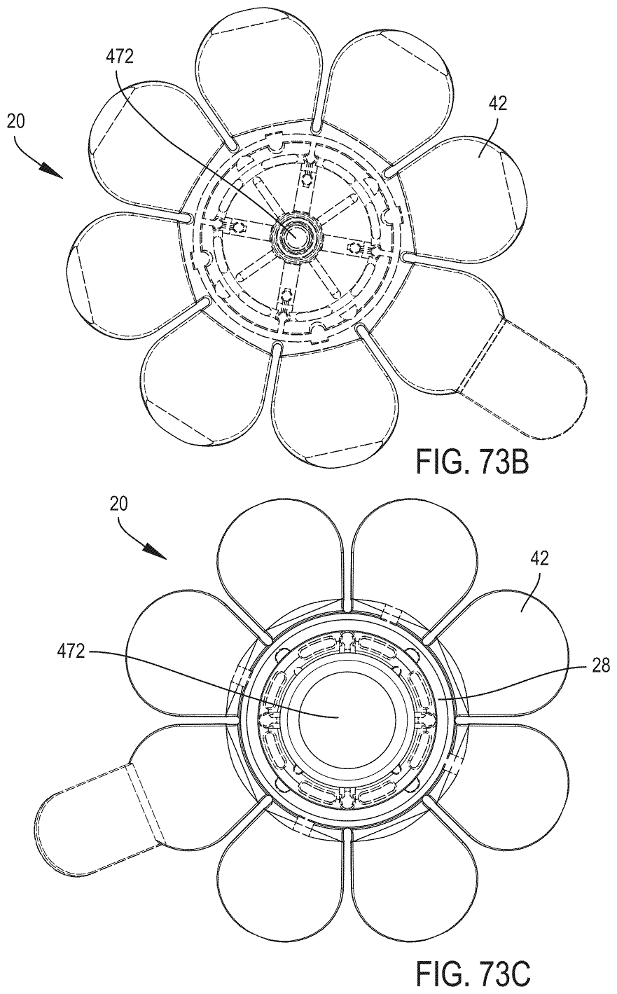

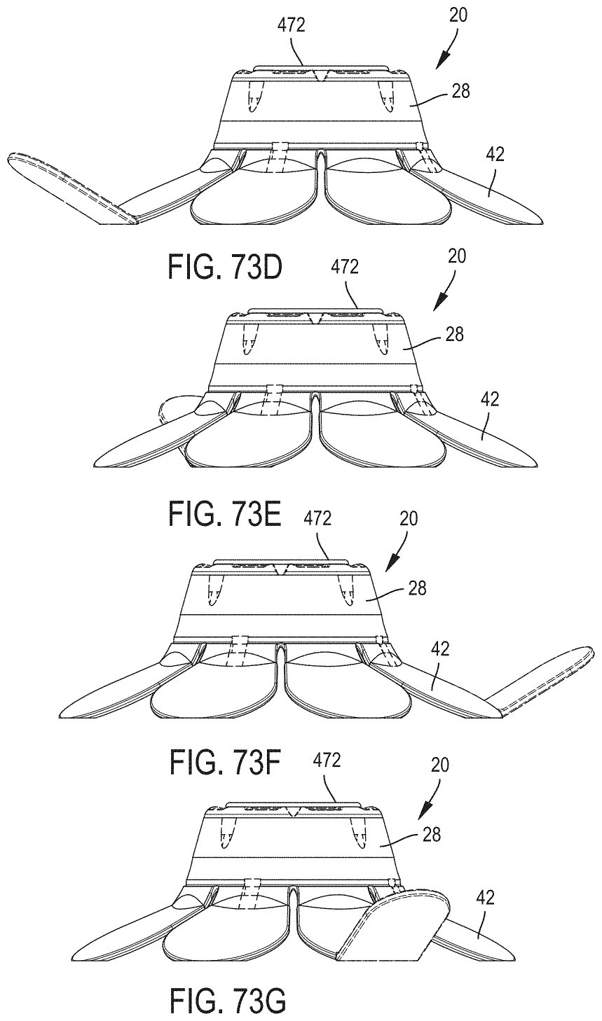

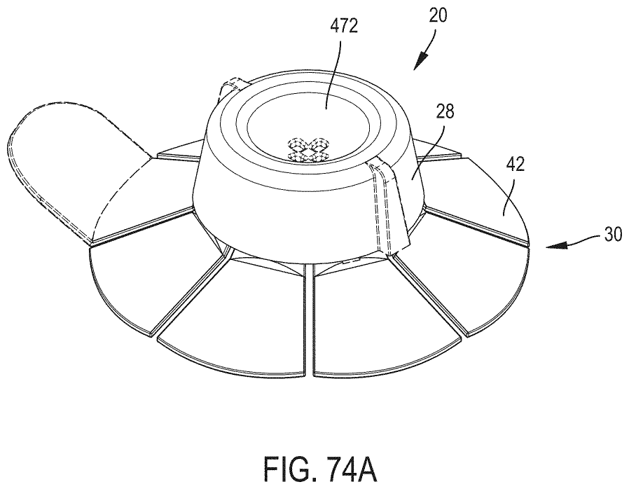

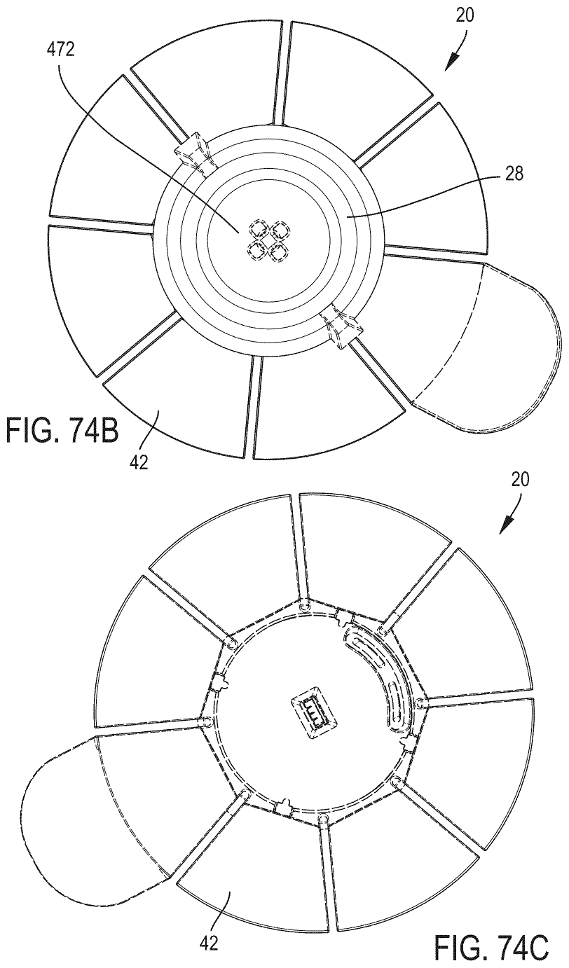

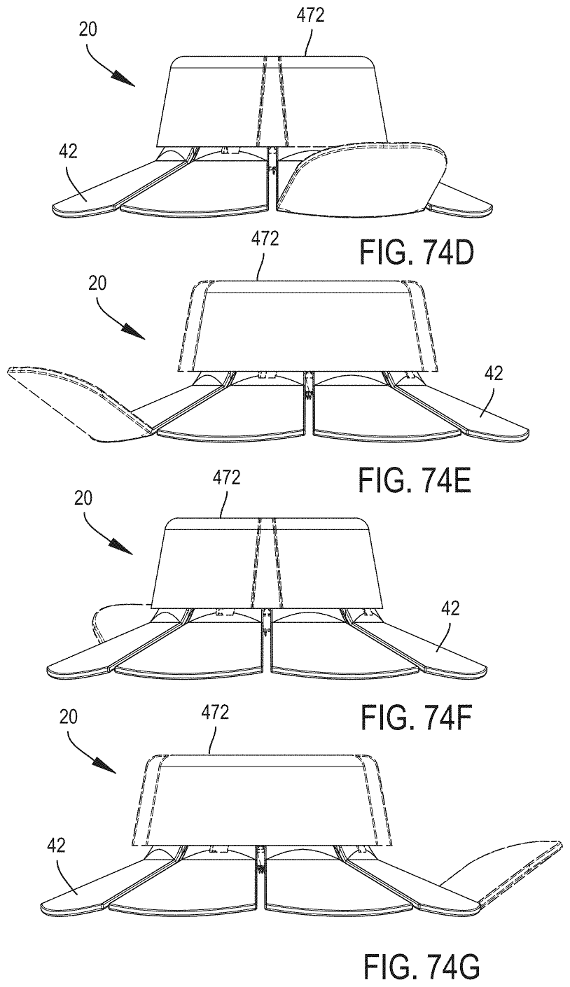

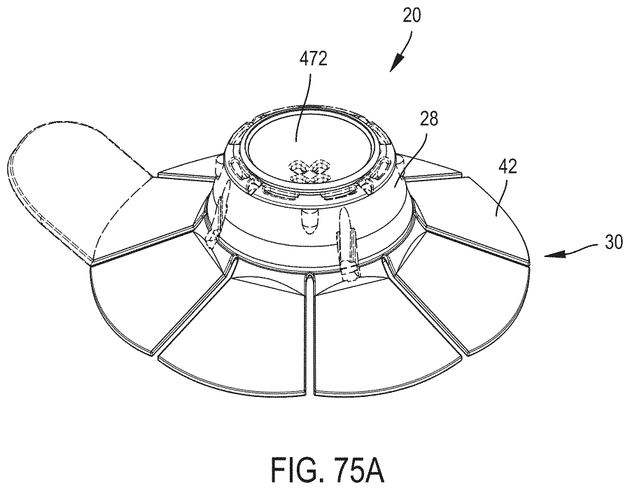

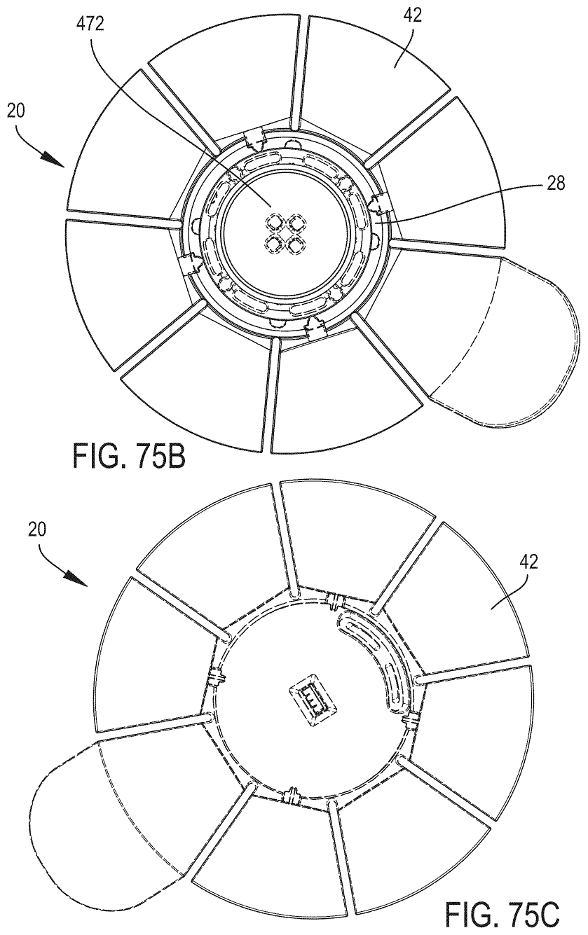

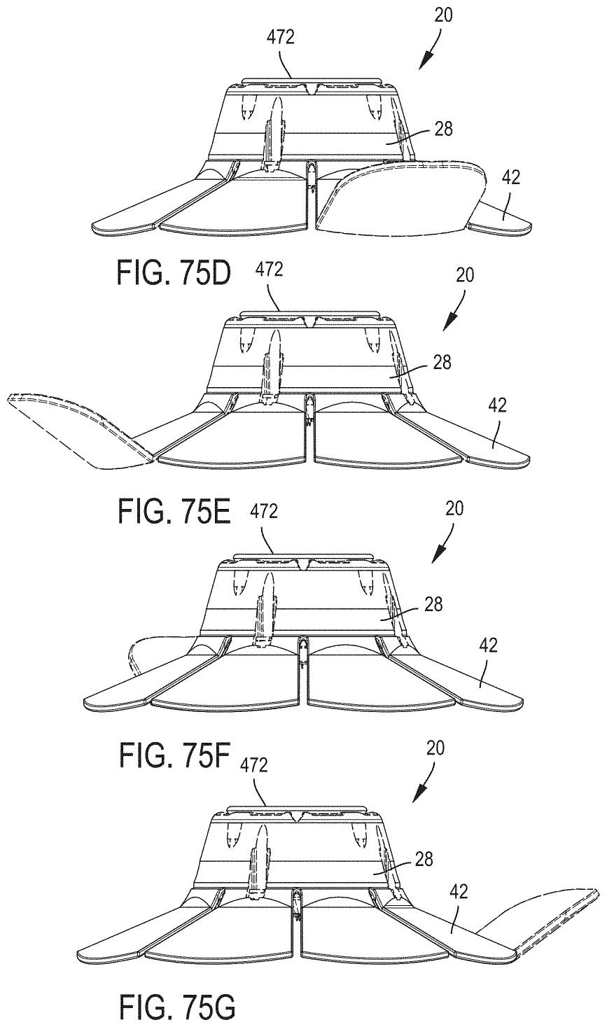

Referring now to FIGS. 73A-73G, various views of a portion of an example delivery device 10 are shown. Various views of example delivery devices 10 are also shown in FIGS. 74A-75G. As shown, compared to typical syringe and needle based injections, delivery devices 10 of the type shown and described herein possess ornamentality which may, for instance, make them more approachable. This may be particularly true in populations with a high prevalence of needle anxiety such as children. In some though not all examples, portions of a delivery device 10 may be shaped and or colored to resemble a flower (e.g. daisy). A depressor body 472 (and perhaps at least part of the center region 28 of a main body 20) may have a first appearance (e.g. color, material, pattern) while at least the petal members 42 of the main body 20 may have a second appearance (e.g. color, material, pattern) which differs from the first. Depressor bodies 472 may, for example, have the appearance of the center portion of a flower. The petal members 42 may have a flower petal like appearance. Certain features may differ or be optional as indicated by dashed lines in FIGS. 73A-75G while still retaining a delivery device 10 or portion thereof having desirable ornamental aspects. Additionally, parts of the delivery devices 10 and portions thereof shown and described in relation to FIGS. 73A-75G possess desirable ornamental aspects in isolation (e.g. depressor bodies 472, main bodies 20, sections of main bodies 20 such peripheral regions 30, petal members 42, sets of petal members 42). Additionally, delivery devices 10 and portions thereof shown and described in relation to FIGS. 73A-75G may be modified to, for example, include greater or fewer petal members 42. In some implementations, petal members 42 may be separated by slits in the peripheral region 30 of the identical to those shown in FIGS. 73A-75G, however, the size of the petal members 42 may differ to accommodate inclusion of the desired number of petal members 42. In such examples, the petal members 42 may have the same cross-sectional shape as those shown, however, that cross-sectional shape may be revolved along a larger or smaller arc depending on the number of petal members 42 desired. The gaps between petal members 42 formed by the slits in the peripheral region 30 may be wider or smaller in some alternative embodiments.

Referring now to FIGS. 76A-76B, a top plan and bottom plan view of an example reservoir 12 are depicted. The example reservoir 12 may, for example, be included in various delivery devices 10 such as any of the exemplary delivery device 10 embodiments described above. As shown, various example reservoirs 12 may include at least one septum 550. A septum 550 may be disposed in an off-center location in the reservoir 12 adjacent a rocker member 526 in certain examples. When the reservoir 12 is assembled, the septum 550 may have a first portion which may be in fluid communication with the interior volume of the reservoir 12. The septum 550 may also include an externally accessible portion. In some embodiments, the reservoir 12 may include a flow channel which is in fluid communication with the main fluid holding volume of the reservoir 12, but is sealed from the exterior environment by the septum 550. The flow channel may extend from a space adjacent the first portion of the septum 550 to the main fluid containing volume of the reservoir 12. The flow channel may be defined by a portion of the disk body 275 and the reservoir portion 271. The reservoir 12 may be filled through the septum 550 (e.g. via a dispensing sharp 570) and fluid may flow through the flow channel (if included) to the main interior cavity of the reservoir 12. Though shown in relation to the embodiment depicted in FIGS. 76A-76B, septums 550 may be included in other example reservoirs 12 described herein.

The example reservoir 12 includes a reservoir portion 271 with a wall 261 having a collapse facilitator (see, e.g., FIGS. 57-58). The wall 261 in the example embodiment includes a number of step regions 259 similarly to as described in relation to FIG. 58. Thus, the wall 261 may have a tiered appearance. The main interior volume of the example reservoir 12 is a step pyramid or ziggurat shaped volume in the example depicted. As shown, the reservoir 12 is in a filled state. The holder 270 includes a stage projection 279 (further described in relation to FIGS. 55A-D) and a rocker member 526 (further described in relation to FIGS. 63A-64B).

In the example shown, a side channel 282 of the reservoir portion 271 has been sealed closed by heat staking the reservoir portion 271 material to the holder 270. The portion of the side channel 282 at the periphery of the flange 284 is sealed against the holder 270 leaving the remaining portion of the side channel 282 open. In alternative embodiments, the reservoir portion 271 may not include a side channel 282. The entire peripheral region of the flange 284 may be coupled to the disk body 275 during manufacture. In certain example embodiments, the wall 261 forming the cavity in the reservoir portion 271 may include an offshoot or a node which extends away from the main portion of the cavity.

Reservoirs 12 including a septum 550 such as that shown in FIGS. 76A-76B may be shipped in an unfilled state. The reservoir 12 may be filled at a pharmacy, hospital, physician's office, vaccination site, or other patient care setting. Alternatively, reservoirs 12 may be filled at a local distribution center from which they may be subsequently disseminated to the surrounding population. The reservoir 12 may be filled temporally proximate use of a delivery device 10. Thus, agent may only be contained in the delivery device 10 for a short period of time (e.g. minutes to weeks). This may allow for reservoirs 12 or delivery devices 10 to be shipped without need for cold chain distribution networks. Additionally, this may help limit need for prolonged agent compatibility testing and facilitate a more nimble response to public health crises.

Referring now also to FIGS. 77-78, when desired, example reservoirs 12 may be filled by establishing fluid communication between an interior fluid holding volume of the reservoir 12 and a filling implement (not shown). In various examples, a filling implement such as a syringe may be used and may include a dispensing sharp 570. The dispensing sharp 570 may be advanced through the septum 550 and fluid may be transferred from the filling implement into the interior volume of the reservoir 12 via the dispensing sharp 570. Any adhesive member 22 (see, e.g., FIGS. 81A-C) on the delivery device 10 may include an open region to allow access to the septum 550 via the dispensing sharp 570 (or the adhesive member 22 may be coupled to the delivery device 10 after filling). Once a desired volume of fluid has been transferred into the reservoir 12, the dispensing sharp 570 may be withdrawn from the septum 550. The septum 550 may be constructed of a self-sealing material such that when the dispensing sharp 570 is withdrawn, the septum 550 provides a robust seal between the interior volume of the reservoir 12 and the external environment. In some examples, prior to transferring fluid into the reservoir 12, a vacuum may be pulled on the reservoir 12 via the filling implement (e.g. by withdrawing the plunger of a syringe). The dispensing sharp 570 may be removed from the septum 550 and any gas sucked out of the reservoir 12 may be expelled from the filling implement. This may help to ensure a minimal volume of gas is present in the reservoir 12 prior to filling.

In certain examples, the reservoir 12 may include a guard which helps to inhibit contact of the reservoir portion 271 with the tip 572 of a dispensing sharp 570. The guard may help keep the reservoir portion 271 in spaced relation to the dispensing sharp 570 during filling of the reservoir 12. The guard may, for example, block a portion of the reservoir portion 271 from displacing into a sharp receiving region of the reservoir 12 where the tip 572 of a dispensing sharp 570 may be disposed during filling.

Referring primarily to FIG. 77, an example embodiment of a septum 550 is depicted. As shown, the septum 550 includes a plug portion 552 and a standoff 554 which may act as a guard. The plug portion 552 may include a first end 556 and a second end 558. The first and second ends 556, 558 may be connected by a stem body 562. The stem body 562 may be narrower (e.g. have a smaller diameter) than either of the first and second ends 556, 558. The first end 556 may be wider (e.g. larger diameter) than the second end 558. The standoff 554 may project from the second end 558. In the example shown, the standoff 554 is shaped substantially as a hemisphere and includes a recessed channel 564. The recessed channel 564 may extend across the width of the standoff 554 forming a canyon type feature in the standoff 554.

Referring primarily to FIG. 78, as shown, the holder 270 may include an aperture 560 which extends through the disk body 275 of the holder 270. The septum 550 may be a fluid tight plug for this aperture 560 when the reservoir 12 is assembled. For example, the septum 550 may be installed in the reservoir 12 by advancing the standoff 554 and first end 556 through the aperture 560. The standoff 544 may have a shape (e.g. a spherical segment) which helps guide the septum 550 into the aperture 560. When installed, the stem body 562 may be disposed within the bore of the aperture 560. The first end 556 may be disposed against the face of the holder 270 from which the stage projection 279 extends. The second end 558 may be disposed within the side channel 282 (or in an offshoot or node projecting from the main cavity of the interior volume of the reservoir 12). Thus the plug portion 552 may establish a fluid tight seal between the exterior environment and the interior volume of the reservoir 12. The stem body 562 of the septum 550 may have a width (e.g. diameter) which is slightly larger than that of the aperture 560 such that the stem body 562 is under compression when the septum 550 is installed within the reservoir 12.

When installed within the reservoir 12, the section of the reservoir portion 271 in which the side channel 282 (or offshoot or node from the main cavity of the reservoir 12) is formed may be inhibited from displacing into the recessed channel 564 by the remainder of the standoff 554. The portions of the standoff 554 adjacent the recessed channel 564 may hold the reservoir portion 271 above the recessed channel 564. Thus, the recessed channel 564 may form a sharp receiving volume within the reservoir 12. The tip 572 of a dispensing sharp 570 may be advanced into the recessed channel 564 while being kept spaced away from the material forming the reservoir portion 271.

In certain embodiments, an adapter 566 may be utilized with any suitable filling implement to ensure that the dispensing sharp 570 is prevented from advancing into the septum 550 beyond a certain distance. The adapter 566 may, for example, couple to a filling implement or hub 568 to which the dispensing sharp 570 is attached. The adapter 566 may extend along a portion of the dispensing sharp 570 shortening the exposed length of the dispensing sharp 570. The adapter 566 may contact or bottom out against the reservoir 12 as the dispensing sharp 570 is introduced into the septum 550 and inhibit further displacement of the dispensing sharp 570 into the septum 550. The adapter 566 may ensure that the tip 572 of the dispensing sharp 570 is limited from displacing out of the recessed channel 564. In alternative embodiments, an adapter 566 may be omitted. The dispensing sharp 570 may have an exposed length (e.g. extending from a hub 568) which is shorter than a height of the septum 550, but longer than a distance between the first end 556 of the septum 550 and the most proximate point of the recessed channel 564. Thus, when inserted, the tip 572 of the dispensing sharp 570 may be disposed within the recessed channel 564.

Referring now to FIG. 79, the example reservoir 12 of FIGS. 76A-78 is depicted assembled into an exemplary delivery device 10. Depending on the embodiment, the reservoir 12 may be filled while installed in a delivery device 10 or may be filled and subsequently installed in a delivery device 10. Where the reservoir 12 is filled separate from the delivery device 10, a user may install the reservoir 12 into the delivery device 10 after filling and then couple an adhesive member 22 to the delivery device 10. Where the delivery device 10 is filled in a pharmacy or the like, a fixture may be provided to assist in positioning the adhesive member 22 and delivery device 10 when coupling the two together.

In certain examples, and referring now also to FIGS. 80A-80B, the main body 20 of the delivery device 10 may include one or more retention tabs 580. The retention tabs 580 may form ledges extending from the interior sidewall of the main body 20 which may be spaced from respective stop surfaces 582 above each retention tab 580. In various examples, the main body 20 may be formed as a monolithic body with the retention tabs 580 and stop surfaces 582 in a single molding operation without use of side actions in the mold. The retention tabs 580 may be created by incorporating bypass shutoffs in the mold. The retention tabs 580 may allow the reservoir 12 to be coupled to the main body 20 via a snap fit. To install a reservoir 12 into the delivery device 10, the reservoir 12 may be displaced against an underside (a side which would be most proximal a patient when the delivery device 10 is in use) of the retention tabs 580 and pressed toward the top surface 250 of the main body 20. The retention tabs 580 may deflect to allow passage of the disk body 275 of the reservoir 12 beyond the retention tabs 580 and into abutment with a stop surface 582. The retention tabs 580 may resiliently restore to hold the reservoir 12 in place within the delivery device 10. Retention tabs 580 and stop surfaces 582 may be incorporated into various delivery devices 10 described herein (e.g. any of those described above). Embodiments including a ridge 290 (see, e.g., FIG. 28) may, for example, be modified to include retention tabs 580 and stop surfaces 528 in place of the ridge 290. Referring now to FIGS. 81A-C, a number of example adhesive members 22 are depicted on exemplary delivery devices 10. As shown, a single adhesive member 22 is included for each of the example delivery devices 10. In alternative embodiments, the adhesive member 22 may be broken into a plurality of individual adhesive members 22. This may facilitate use of different adhesives or perhaps leaving certain petal member 42 devoid of adhesive. As shown, each adhesive member 22 may include a plurality of slits 43 extending radially inward from a periphery of the adhesive member 22 so as to create petal portions which align with the petal members 42 of the main body 20. The adhesive member 22 may also include a central aperture 49 through which the delivery sharp(s) 72 of the delivery device 10 may access a patient.

The shape and size of the central aperture 49 may have an effect on bleb formation resulting from delivery when a delivery device 10 is used. Additionally, the shape and size of the central aperture 49 may play a role in helping to facilitate certain shallow deliveries or shallow deliveries into skin having certain characteristics. In various exemplary delivery devices 10, it may be desirable that the central aperture 49 have a cross sectional area which is 60-100% the area of the footprint of the holder 270. It may also be desired that the central aperture 49 be shaped such that at least a portion of the adhesive member 22 is attached to a portion of a holder 270 or other rigid portion of the reservoir 12. In certain examples, the cross-sectional area of the central aperture 49 may be greater than 0.13 in2. In certain examples, the cross-sectional area of the central aperture 49 may be in a range of 0.13 in2 to 0.5 in2 (e.g. about 0.3 in2).

Additionally, it may be desired that the central aperture 49 be wider in certain directions compared to others. For instance, each delivery sharp 72 (e.g. one or more microneedle) may tend to dispense fluid in an ejection direction which extends from the outlet of the respective delivery sharp 72 (e.g. along the axis of the lumen of the delivery sharp 72). It may be desired that the central aperture 49 have a larger or increased width in a direction which aligns or substantially aligns with the ejection direction. For example, the greatest width (or at least a comparatively large width portion) of the central aperture 49 may be along a direction that is parallel to a plane that includes the ejection direction. Using a delivery device 10 including one or more microneedle similar to that shown in FIG. 2, an increased width portion of the central aperture 49 may be aligned with the front to back (distal side 15 to back facing edge 23, may also be referred to herein as length) direction or line of symmetry of the microneedle. For example, the central aperture 49 could be obround and be widest in a direction parallel to the front to back direction of the microneedles. This may help to create a more diffuse shallow (e.g. intradermal) injection as opposed to a concentrated bleb. This may, in turn, be desirable as it may help to increase the effectiveness of the injection. For instance, a more diffuse intradermal injection of a vaccine may expose more of the immune related cells in the intradermal region to the vaccine potentially augmenting the immune response.

Referring now primarily to FIG. 81A, the central aperture 49 may generally be a round (e.g. circular) aperture with the exception of a number of inwardly extending teeth or spokes 51 of adhesive member 22 material. In the example embodiment, the adhesive member 22 includes a central aperture 49 with four spokes 51 spaced at regular angular increments from one another. The number of spokes 51 may differ and the spacing of the spokes 51 may be irregular in certain examples. The spokes 51 may be disposed such that the central aperture 49 has a comparatively large width in a direction aligned with the ejection direction. Though the central aperture 49 may have a comparatively large width in this direction, this does not preclude other wide regions of equal, lesser, or perhaps even greater width. In the example shown, the central aperture 49 is about equal in width when measured in a direction perpendicular to the front to back direction of the microneedles. In certain examples, spokes 51 may assist in attachment to a holder 270 or other rigid reservoir 12 portion. Thus, the adhesive member 22 may be firmly attached to both the main body and the holder 270 for example. In certain examples, the spokes 51 may be the only portion of the adhesive member 22 which is adhered to the holder 270.

Referring now primarily to FIG. 81B-C, in certain examples, the central aperture 49 may include notches 53 which extend outwardly from the periphery of the rest of the central aperture 49. The notches 53 may be included to widen the central aperture 49 where desired. Though rectangular notches 53 are included in the examples, the shape of the notches may differ in alternative embodiments. The notches 53 could be any suitable polygonal shape or could be round for example.

Various main bodies 20, as well as petal members 42, central regions 28, top surfaces 250, ridges 290, retention tabs 580 thereof are described above. It shall be understood that various delivery devices 10 described herein are exemplary. Any main body 20 described above may be used in any of the above delivery devices 10. Similarly, any main body 20 features described above may be incorporated into any of the main bodies 20 described above. Various adhesive members 22 are described herein. Any adhesive members 22 described above may be utilized in any of the delivery devices 10 described above. Any of the delivery sharps 72 or sharp bearing bodies 26 described herein may be used in any of the delivery devices 10 described above. Any of the dispensing assemblies 480 or portions thereof (e.g. bias members 470, depressor bodies 474) described herein may be used in any of the delivery devices 10 described above. Various reservoirs 12 and features thereof (e.g. holders 270, rocker members 526, reservoir portions 271, reservoir walls 261, stage projections 279, flow restrictors 524) are described above and may be used in any of the delivery devices 10 described above.

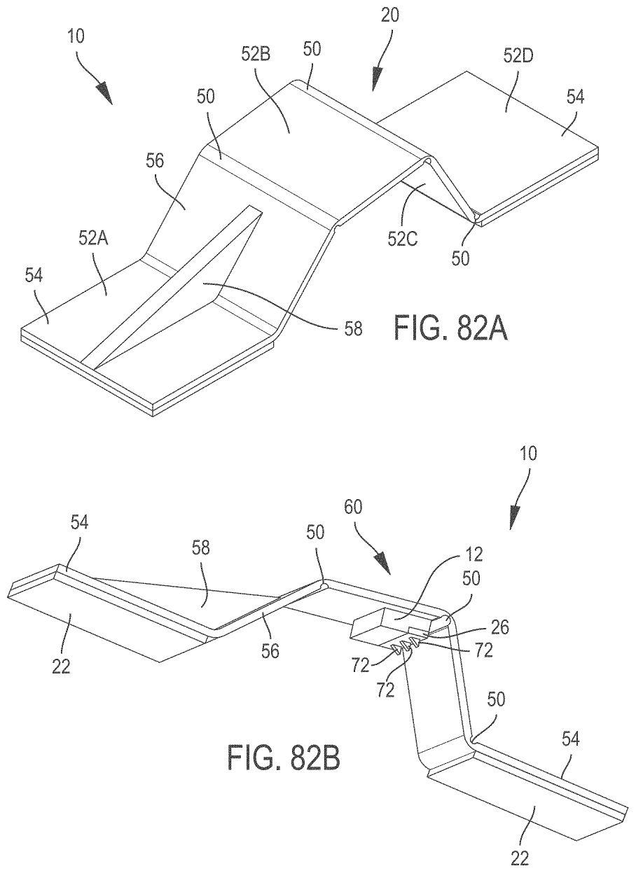

Referring now to FIG. 82A and FIG. 82B, another example embodiment of a delivery device 10 is depicted. As shown, the delivery device 10 includes a main body 20 and a reservoir 12. The reservoir 12 includes a sharp bearing body 26 which includes a one dimensional array of three delivery sharps 72. Other embodiments may include any suitable number of delivery sharp(s) 72 arranged in any desired pattern. The main body 20 of the delivery device 10 may have a polygonal (e.g. rectangular) footprint and may be generally formed as a strip of material to which the reservoir 12 is attached. In the example embodiment, the main body 20 includes a number of living hinges 50 which are formed integral with the rest of the main body 20. The living hinges 50 may each extend across a portion of the main body 20 and may partition the main body 20 into a number of panels 52A-D. The main body 20 may be injection molded.

The panels 52A-D may include a pair of opposed end panels 52A, D. Each of the end panels 52A, D may include a planar portion 54. The planar portions 54 may be parallel or coplanar. In the example, the planar portions 54 are coplanar. One of the end portions 52A may include an angled projection 56 which extends from planar portion 54. The angled projection may extend from the planar portion 54 such that an obtuse angle is formed between the distal faces of the planar portion 54 and angled projection 56. The angled projection 56 may be resilient and resist deflection or bending so as to extend at a fixed angle with respect to the planar portion 54. In some examples, a buttress 58 may be included and may extend from the planar portion 54 to the angled projection 56 to aid in preventing displacement of the angled projection 56 with respect to the planar portion 54. Each of the planar portions may have a proximal face which is at least partially covered in adhesive 22.

The panels 52A-D may also include at least two intermediate panels 52B, C which may extend between and couple together the end panels 52A, D. One of the panels 52B may be coupled to an end of the raised projection 56 via one of the living hinges 50. The other of the intermediate panels 52C may be coupled to the planar end panel 52D via another of the living hinges 50. Each of the intermediate panels 52B, C may be coupled to one another via a living hinge 50 so as to form a linkage 60 between the end panels 52A, D.

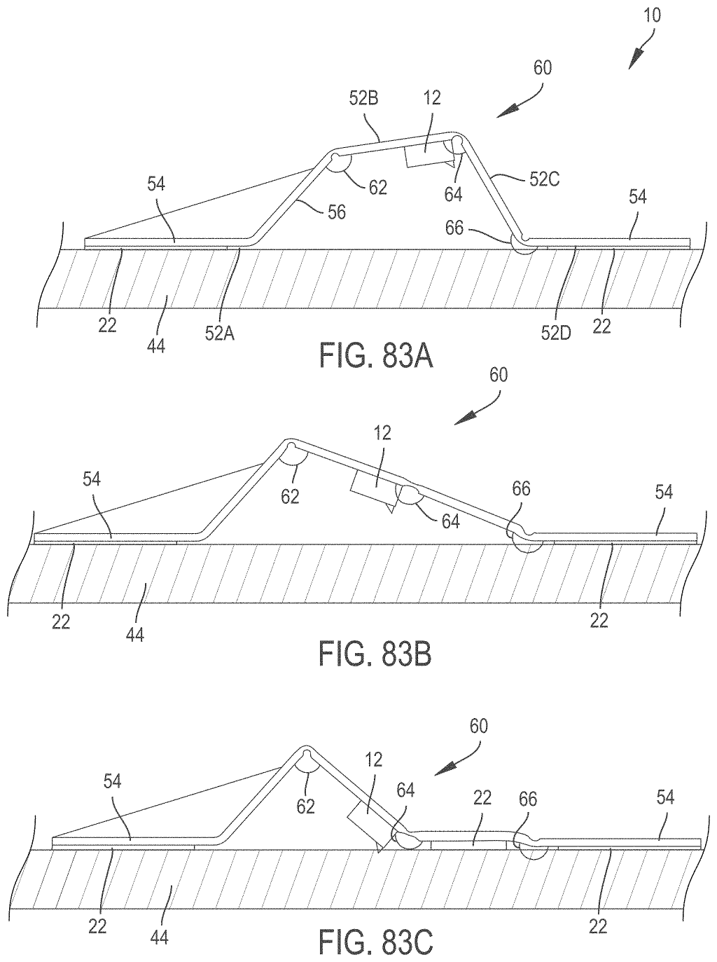

Referring now to FIGS. 83A-83C, the linkage 60 may be displaceable between a raised position (see FIG. 83A), through a center position (see FIG. 83B) and into an over center position (see FIG. 83C). The linkage 60 may be in the raised position when the delivery device 10 is in the storage state. In the raised position, the intermediate panel 52B connected to the raised projection 56 may extend from the raised projection 56 at an angle 62 (angles 62, 64, 66) measured between proximal faces of recited components). The angle 62 may be selected such that the intermediate panel 52B becomes progressively more distant from the plane of the planar portions 54 as distance from the raised projection 56 increases. In the example shown, angle 62 is an obtuse angle when the linkage 60 is in the raised position. The intermediate panels 52B, C may also be disposed at an angle 64 to one another. This angle 64 is also an obtuse angle in the example embodiment when the linkage 60 is in the raised position. The intermediate panel 52C and end panel 52D may form an angle 66 with respect to one another which may be a reflex angle when the linkage 60 is in the raised position. Additionally, in the raised position each of the end panels 52A, D may be at a closest distance to one another.

The delivery device 10 may be applied to the skin 44 over an infusion site in the storage state with the linkage 60 in the raised position. This may fix the end panels 52A, D such that they are substantially constrained to the plane of the skin patch to which they are adhered. Application of downward pressure against the linkage 60 may displace the linkage 60 from the raised position toward the center position. As this occurs, the angle 64 between the two intermediate panels 52B, C may increase. The angle 62 between the raised projection 56 and intermediate panel 52B and the angle 66 between end panel 52D and intermediate panel 52C may decrease. To accommodate the change in the angle 64 between the two intermediate panels 52B, C the end panels 52A, D may spread apart. When the linkage 60 reaches a center position (see FIG. 83B) the angle 64 may be 180° and the end panels 52, D may be at a greatest distance from one another. The skin 44 may be stretched and rendered taught as the end panels 52A, D displace apart.

As the linkage 60 is further displaced the linkage 60 may enter an over center state. The elasticity of the stretched skin 44 may exert a restoring force which tends to drive the end panels 52A, D toward one another. Thus, once the linkage 60 is displaced through the center position the linkage 60 may automatically be displaced to an over center position at an end of the displacement range of the linkage 60. When the linkage 60 is displaced into this over center position, the delivery device 10 may be transitioned into the delivery state. As the linkage shifts to the over center position shown in FIG. 83C, the distance between the end panels 52A, D may decrease. The distance between the end panels 52A, D may still, however, be greater than the distance between the end panels 52A, D when the linkage 60 is in the raised position. In the over center position at the end of the displacement range, the angle 62 between the raised projection 56 and intermediate panel 52B may be about 90° (e.g. 80°-110°). The angle 64 between the intermediate panels 52B, C may be a reflex angle. Thus, the linkage 60 may be partially inverted with respect to its position in the raised position. The angle 66 between intermediate panel 52C and end panel 52D may be substantially 180°. As shown, when the linkage 60 reaches the over center position at the end of its displacement range a proximal face of the intermediate panel 52C may contact the skin 44.

When the delivery device 10 is in the delivery state (see FIG. 83C), the delivery sharp(s) 72 may be pressed into the skin 44 so as to puncture the skin 44 and establish fluid communication with a delivery destination in the patient. The angle 64 between the intermediate panels 52B, C in the over-center delivery position (see FIG. 83C) may be selected to cause the delivery sharp(s) 72 to pierce into the skin 44 at a prescribed angle (e.g. 45° to the skin 44 surface). The angle 64 may be selected to be in the range of 30° to 60°. Alternatively, the angle 64 may be established by setting within a desired range the ratio of the shortest distance between the angle 62 and the surface of the skin 44 in the over-center delivery position (see FIG. 83C) to the length of the intermediate panel 52B. The reservoir 12 may also be pressed against the skin 44 when the delivery device 10 is in the delivery state 10. This may pressurize the reservoir 12 and force fluid out of the reservoir 12 through the delivery sharp(s) 72 and into the patient. Restoring force exerted by the stretched skin 44 may serve to supply a continuous pressure against the reservoir 12 and aid in ensuring that the reservoir 12 is completely depleted as the delivery occurs.

In some embodiments, the proximal face of the intermediate panel 52C may be covered at least partially in adhesive 22 (see, e.g., FIG. 83C). When the linkage 60 reaches the over center position at the end of the displacement range, the adhesive 22 may hold the linkage 60 in place. Additionally, in some embodiments, the main body 20 may include at least one force limiter. For example, at least one of the panels 52A, D may include a strain relief flexure. In the example embodiment, this flexure may bend in the event that the force required to stretch the skin 44 exceeds a threshold. Bending of the flexure may cause the linkage 60 to snap through the center position and enter an over center position such that stretching of the skin 44 is halted. This may be desirable as it may help to mitigate potential discomfort during an injection due to excessive tensioning of the skin 44 Likewise, this may be helpful in certain patient populations as skin characteristics vary significantly across potential patients.

In an embodiment, one of the intermediate panels 52B, 52C, for example the intermediate panel 52C, may be implemented as a flexure or including at least one flexure that incorporates a gap and at least one bias member. The gap may be urged to a widened state by the bias member (which may, in some embodiments, be formed integrally with and of the same material as the panel 52B, C). Applying sufficient pressure to the bias member may overcome the bias member and allow the flexure to give. Thus, an intermediate panel 52B, 52C may be formed so as to have a variable length which decreases as force exceeds some predefined threshold.

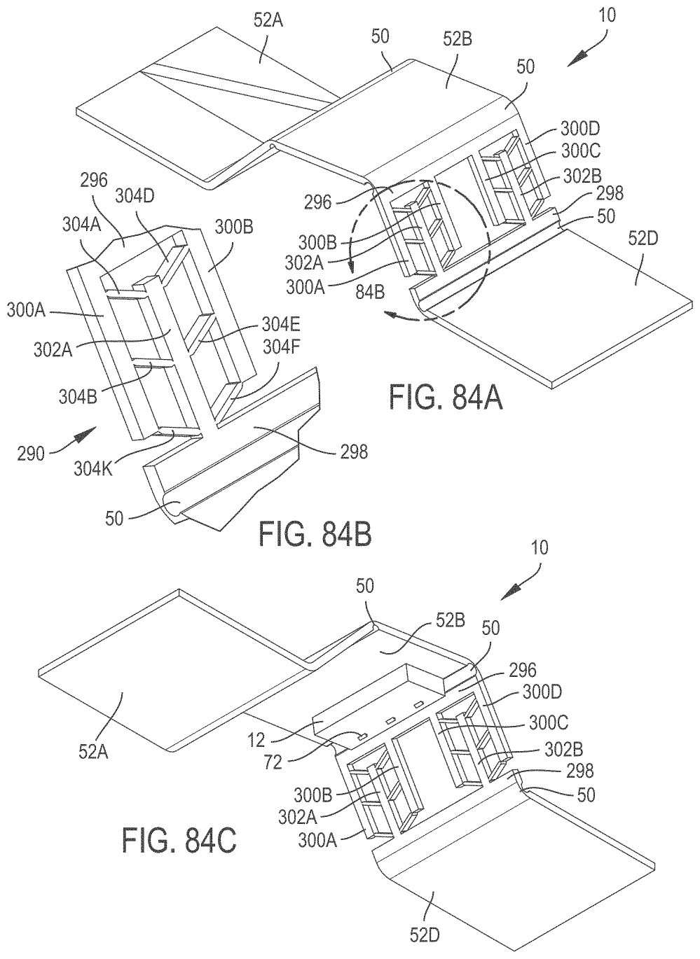

The intermediate panel 52C (though any intermediate panels 52B, 52C may include such features) may be implemented as or to include at least one lattice-structured flexure 290, as shown in FIGS. 84A-84B. The flexure(s) 290 may be formed by injection molding. The intermediate panel 52C may include a first member 296 adjacent the living hinge 50 connection to the other intermediate panel 52B. The first member 296 may have at least one support arm 300. In the example shown, there are four support arms 300A-D extending from the first member 296 at substantially ninety degree angles toward a second member 298 of the intermediate panel 52C. The second member 298 may be adjacent the living hinge 50 connection to the end panel 52D. The second member 298 may be disposed parallel to and opposing the first member 296 and have at least one arm 302. In the example shown there are two arms 302A-B extending from the second member 298 at substantially ninety degree angles toward the first member 296. Each of the two arms 302A-B may be positioned substantially parallel to and extend between a respective pair of the four support arms 300A-D. Each of the two arms 302A-B may be coupled to at least one of the associated two support arms 300A-B, 300C-D disposed on each side of the respective arm 302A-B by at least one buttress 304 (e.g., three buttresses 304). In the example, each of the two arms 302A, B is connected to each of the associated support arms 302A-B. Only six of twelve buttresses 304A-F are shown in FIG. 84B for ease of illustration. Applying pressure that exceeds a threshold level will cause the gaps between the first and second members 296, 298, the support arms 300A-D, the arms 302A-B, and the buttresses 304A-F to at least partially close. The number of buttresses 304A-F may be altered to adjust the threshold at which that flexure 290 gives way. Additionally or alternatively, the amount of material or thickness of the buttresses 340A-F may be adjusted for this purpose. The buttresses 304A-F may be disposed substantially parallel with each other and extend from the respective arm 302A-B at acute angles relative to the first member 296. The buttresses 304A-F may couple to the respective support arms 300A-D at obtuse angles relative to the first member 296. The flexures 290 are shown by way of non-limiting example and may be incorporated using any suitable shapes, angles, and/or numbers of structures and/or parts.

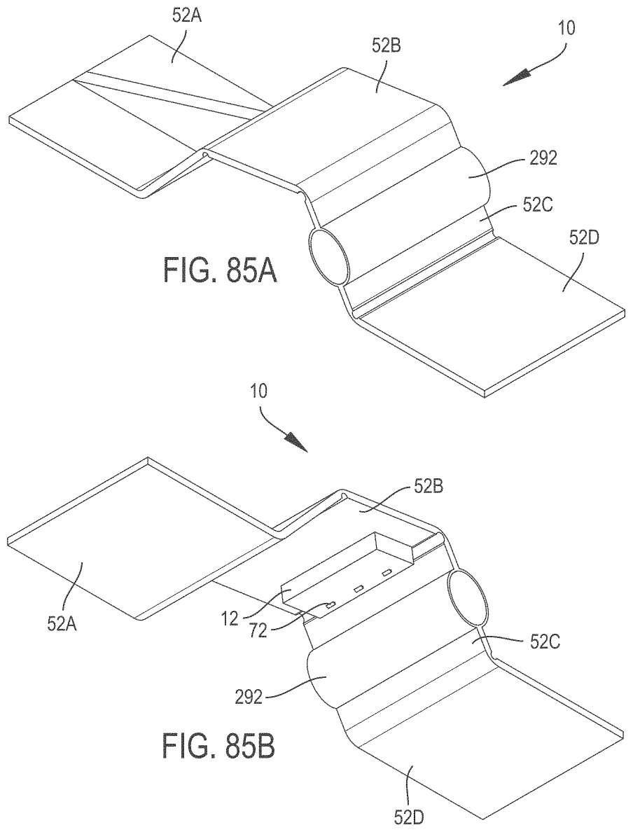

In another embodiment the intermediate panel 52C may incorporate at least one flexure 292 which may be in the form a squishable body that may deform when a threshold force applied to the body is exceeded. The flexure 292 may be round and hollow in an embodiment. As shown in FIGS. 85A-85B, the exemplary flexure 292 may be roughly cylindrical in shape. It would be understood that the flexure 292 shown is one of many possible examples and need not be limited to the structure or shape shown. The flexure 292 may be integral to the intermediate panel 52C and may be formed in an injection molding process that forms the main body 20 of the delivery device 10.

In some embodiments, at least a portion of the main body 20 may plastically deform after the delivery device 10 is transitioned from the storage state to the delivery state. For example, one of the living hinges 50 may be plastically deformed. Alternatively, one or more of the living hinges 50 may break when an attempt to transition the delivery device 10 back to the storage state from the delivery state is made. Thus, the transition from the storage state to the delivery state may be rendered irreversible and reuse of the delivery device 10 may be prevented. In some examples, the adhesive 22 may be selected so as to bond more aggressively to the skin 44 than the material forming the main body 20. Thus, upon removal of the delivery device 10, the adhesive 22 may peel off of the delivery device 10. This may also aid in inhibiting reuse of the delivery device 10. In embodiments described herein where the adhesive 22 peels off the delivery device 10, the adhesive 22 may include a pull tab or similar feature which would facilitate subsequent removal from the skin 44.

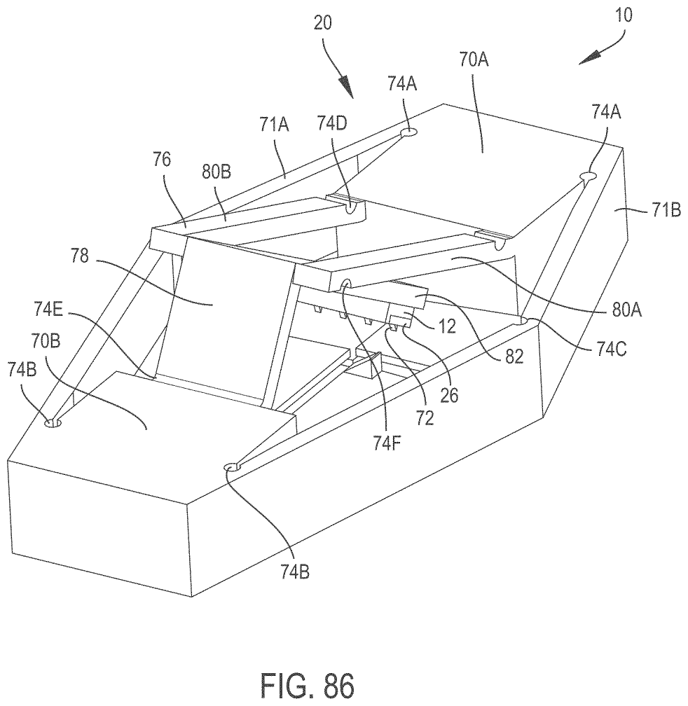

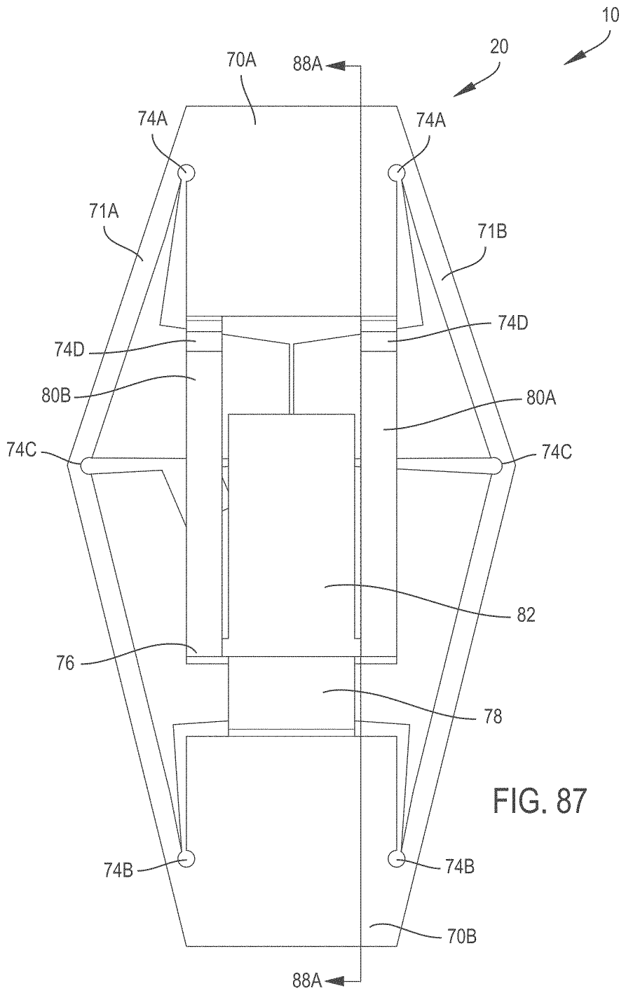

Referring now to FIGS. 86-87, another example embodiment of a delivery device 10 is depicted. As shown, the delivery device 10 includes a main body 20 and a reservoir 12. The reservoir 12 includes a sharp bearing body 26 which includes a one dimensional array of delivery sharps 72. Other embodiments may include any suitable number of delivery sharp(s) 72 arranged in any desired pattern. The main body 20 of the delivery device 10 may have a polygonal footprint which may change from a first polygonal shape (e.g. a hexagonal shape) to a second polygonal shape (e.g. a rectangular shape) when the delivery device 10 is transitioned from a storage state to a delivery state.

The main body 20 may include a first and second end block 70A, B. The end block 70A, B may be disposed in opposition to one another. The end blocks 70A, B may be spaced apart and connected together by a set of side panels 71A, B and a bridge 76. The side panels 71A, B may each have a first end which is connected to the first end block 70A by a hinge 74A. The side panels 71A, B may also each have a second end opposed to the first end which is connected to the second end block 70B by a hinge 74B. Each of the side panels 71A, B may also include an intermediate hinge 74C which may be disposed in an intermediate region of the side panels 71A, B between the first and second end of each side panel 71A, B.

Similarly to the side panels 71A, B, the bridge 76 may have a first end connected to the first end block 70A by a hinge 74D and a second end opposite the first which is connected to the second end block 70B by another hinge 75E. The bridge 76 may additionally include an intermediate hinge 74F disposed between the first and second ends of the bridge 76. The bridge 76 may include a panel body 78 and a set of strut members 80A, B. the strut members 80A, B may be connected to the panel body 78 via the intermediate hinge 74F of the bridge 76. The bridge 76 may also include an arm member 82. The arm member 82 may be disposed between two strut members 80A, B and may extend toward a proximal face of the end blocks 70A, B. In the example embodiment, the arm member 82 extends from an end of the panel body 78 adjacent the intermediate hinge 74F of the bridge 76. The reservoir 12 may be coupled to the proximal face of the arm member 82 at an end of the arm member 82 opposite the panel body 78.

In the example embodiment shown in FIGS. 86-87, the hinges 74A-F are depicted as living hinges. In alternative embodiments, at least one of the hinges 74A-F may be conventional hinges and the main body 20 may be constructed as an assembly of a plurality of components which are coupled to one another via the hinges 74A-F. In still other examples, the main body 20 may be constructed of at least two components. Instead of using a hinge to join the components, the two or more components of the main body 20 may be joined together via welding, heat bonding, solvent bonding, etc.

The side panels 71A, B of the delivery device 10 may be displaceable through a displacement range to transition the delivery device 10 between a storage state (shown in FIGS. 86-87) to a delivery state. In the storage state, the side panels 71A, B of the delivery device 10 may be in an outwardly bowed position. The side panels 71A, B may be bent at the intermediate hinges 74C of each of the side panes 71A, B such that the side panels 71A, B may assume this outwardly bowed position. Some pivoting of the side panels 71A, B at the hinges 74A, B connecting the side panels 71A, B to the end blocks 70A, B may also occur to allow the side panels 71A, B to be placed in the outwardly bowed position.

A pinching force which urges the side panels 71A, B toward one another may be exerted against the side panels 71A, B to displace the side panels 71A, B from the outwardly bowed position to a straightened position. It should be understood that the straightened position need not necessarily be a position in which the side panels 71A, B each extend along straight line. In some examples, the straightened position may be a position in which the side panels 71A, B are less outwardly bowed than in the outwardly bowed position.

The bridge 76 may also be displaceable through a displacement range to transition the delivery device 10 between a storage state (shown in FIGS. 86-87) to a delivery state. In the storage state, the bridge 76 may be in a raised state in which at least the delivery sharp(s) 72 of the reservoir 12 coupled to the arm member 82 are disposed above the proximal faces of the end blocks 70A, B. The bridge 76 may be bent at the intermediate hinge 74F such that the panel body 78 and struts 80A, B extend upward from the end blocks 70A, B and away from the proximal face of the end blocks 70A, B. Some pivoting of the panel body 78 and struts 80A, B at the hinges 74D, E connecting the bridge 76 to the end blocks 70A, B may also occur to allow the bridge to assume the raised position. A force normal to the proximal faces of the end blocks 70A, B may be applied to the bridge 76 to displace the bridge from the raised position to a lowered position at the opposite end of the bridge's 76 displacement range.

In various embodiments, actuation of the bridge 76 through its displacement range may transition the delivery device 10 from the storage state to the delivery state. Additionally, actuation of the side panels 71A, B from the outwardly bowed state to the straightened state may transition the delivery device 10 from the storage state to the delivery state. Actuation of the bridge 76 through its displacement range may result in displacement of the side panels 71A, B through their displacement range since the bridge 76 and side panels 71A, B are coupled to one another through the end blocks 70A, B Likewise, actuation of the side panels 71A, B through their displacement range may result in displacement of the bridge 76 through its displacement range.

It may be left up to the user whether actuation of the side panels 71A, B or bridge 76 is used to transition the delivery device 10. Alternatively, whether the bridge 76 is actuated or the side panels 71A, B are actuated may depend upon the patient population to which the user belongs. For example, actuation of the bridge 76 may result in a greater amount of pressure being exerted against the arm member 82. This may aid in ensuring puncture of the delivery sharp(s) 72 into the skin. Thus, it may be desirable that patient populations with certain skin characteristics be instructed to actuate the delivery device 10 via the bridge as opposed to the side panels 71A, B.

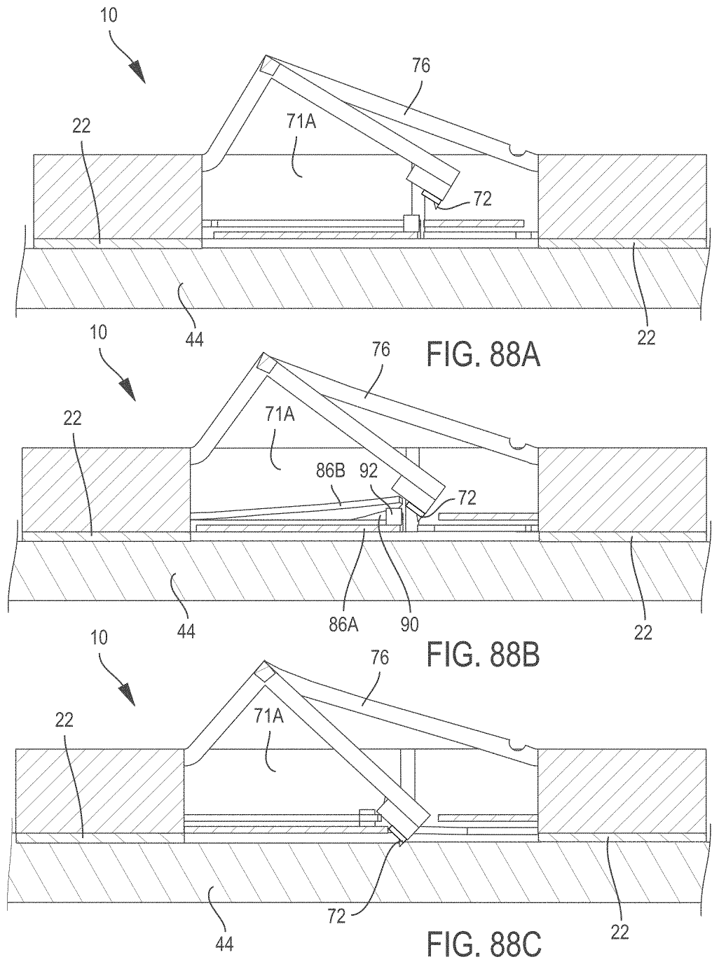

Referring now also to FIGS. 88A-88C, with the side panels 71A, B in the outwardly bowed position and the bridge 76 in the raised position (see FIG. 88A), the end blocks 70A, B may be at a first distance from one another. As the side panels 71A, B are displaced toward the straightened position and the bridge 76 is displaced to the lowered position (see FIGS. 88B-88C), the end blocks 70A, B may be displaced apart from one another. As the delivery device 10 may be attached to the skin 44 via adhesive 22 included on the end blocks 70A, B, the spreading of the end blocks 70A, B may cause the skin 44 to be stretched and rendered taught. This may help to facilitate piercing of the skin 44 by the delivery sharp(s) 72 included on the reservoir 12. As shown in FIG. 88C, when the side panels 71A, B reach the straightened position and the bridge 76 reaches the lowered position, the delivery sharp(s) 72 may puncture the skin 44 and the delivery device 10 may be in the delivery state. The reservoir 12 may be compressed between the skin 44 and the arm member 82 to drive fluid out of the reservoir 12 and into the patient. The reservoir 12 may collapse as delivery occurs.

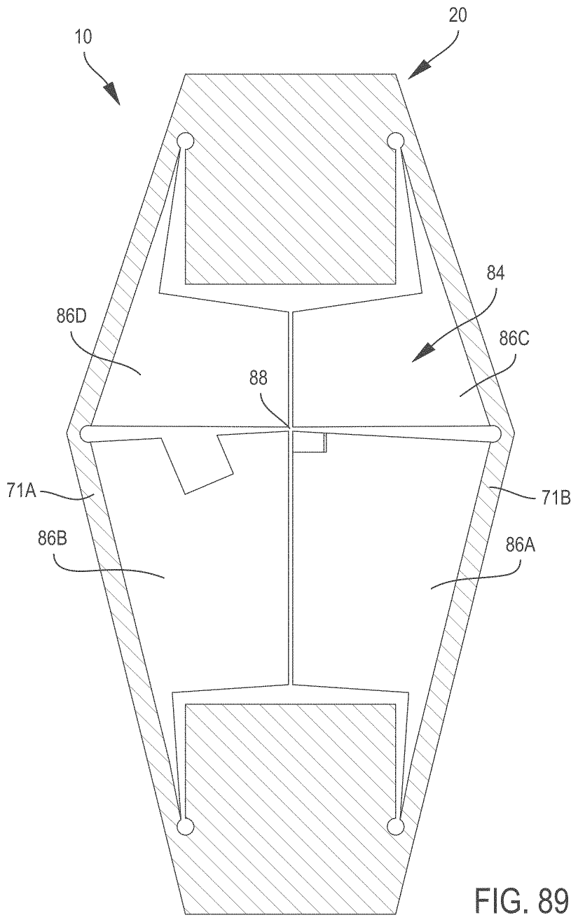

Referring now also to FIG. 89, a cross section of the delivery device 10 in FIGS. 86-87, a delivery device 10 may include an iris assembly 84 in certain examples. The iris assembly 84 may include a set of iris panels 86A-D which may define an aperture 88 which is variable in size from a closed to a fully open state. The iris panels 86A-D may extend from each of the side panels 71A, B in a direction toward the opposite side panel 71A, B. In the example embodiment, two iris panels 86A-D extend from each side panel 71A, B and are disposed on either side of the intermediate hinge 74C, D of each side panel 71A, B. As the side panels 71A, B are displaced from an outwardly bowed position to a straightened state, the iris panels 86A-D may adjust the aperture 88 such that the aperture 88 provides an opening for the delivery sharp(s) 72 of the reservoir 12 to pass through. As shown in FIG. 89, the aperture 88 may be substantially closed when the delivery device 10 is in the storage state. Such an iris assembly 84 may thus serve as a guard which may aid in preventing inadvertent contact with the delivery sharp(s) 72 during handling of the delivery device 10.

In the example embodiment, one of the iris panels 86A includes a latch projection 90. Another of the iris panels 86B includes a latch catch 92 which may be formed as a notch in that iris panel 86B. The latch projection 90 is ramped. Thus, as the iris panels 86A, B are displaced toward one another, iris panel 86B may deflect and ride up the ramp of the latch projection 90 (see, e.g., FIG. 88B). Upon the latch catch 92 aligning over the latch projection 90, the iris panel 86B including the latch catch 92 may restore to an undeflected state and the latch catch 92 may snap into engagement with the latch projection 90. This may lock the delivery device 10 in the delivery state. The snapping action of the iris panel 86B may generate tactile sensation perceptible, for example, via a user's finger tips. Alternatively or additionally, the snap may generate an audible click or slap. Thus, the delivery device 10 may provide an audible and/or tactile indication that the delivery device 10 has been transitioned to the delivery state. Engagement of the latch projection 90 and latch catch 92 may also aid in inhibiting reuse. As discussed in relation to other embodiments herein, the adhesive 22 may be selected so as to bond more aggressively to the skin 44 than the material forming the main body 20. Thus, upon removal of the delivery device 10, the adhesive 22 may peel off of the delivery device 10. This may also aid in inhibiting reuse of the delivery device 10.

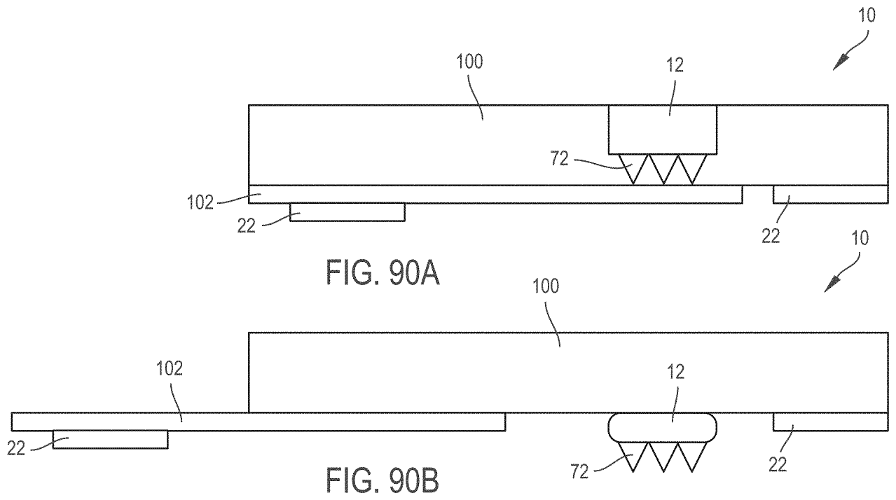

Referring now to FIGS. 90A-90B, another exemplary embodiment of a delivery device 10 is depicted. The delivery device 10 may include a first portion 100 and a second portion 102. One of the first and second portion 100, 102 may translationally displace with respect to the other of the first and second portion 100, 102 to transition the delivery device 10 from a storage state (see FIG. 90A) to a delivery state (see FIG. 90B). In some embodiments, only a part of the first or second portion 100, 102 may translationally displace with respect to the other. For example, one of the first or second portions 100, 102 may stretch and/or elongate. In certain examples, the transition to the delivery state may be reversible, though in other embodiments the transition may be a one-way transition that is irreversible. For example, a latch, lock, or other coupling may be engaged to hold the first and second portion 100, 102 in the delivery state or prevent the first and second portion 100, 102 from returning to the storage state. Alternatively, the first and second portion 100, 102 may be bonded together when the delivery device 10 is transitioned into the storage state. Destruction of a portion of the delivery device 10 may be required to disengage the coupling or bonding between the first and second portion 100, 102 once the delivery device 10 is transitioned to the delivery state. This destruction may render the delivery device 10 inoperative. This may inhibit reuse as well as provide a user perceptible (e.g. visual) indication that the delivery device 10 has been used.

The proximal face of each of the first and second portion 100, 102 may be at least partially covered with adhesive 22. The adhesive 22 may serve to couple the first and second portion 110, 102 to a skin surface at an infusion site on a patient. The delivery device 10 may be adhered to the skin when delivery device 10 is in the storage state and then may be transitioned to the delivery state. As the transition occurs, an adhesive bearing section of the first portion 100 may be displaced with respect to an adhesive bearing section of the second portion 102. Thus, the distance between these adhesive bearing sections may be increased so as to stretch or spread the underlying skin. This may be desirable as the skin may be rendered taught facilitating piercing of the skin by at least one delivery sharp 72 of a reservoir 12 included in the delivery device 10.

Transition of the delivery device 10 to the delivery state may also result in a proximal displacement or lowering of the delivery sharp(s) 72 toward and into the skin. In embodiments where the delivery sharp(s) 72 are coupled to the reservoir 12, the reservoir 12 may also be proximally displaced. In some examples, the reservoir 12 may be compressed between the skin surface and a section of one of the first and second portions 100, 102 when the delivery device 10 is transitioned from the storage state to the delivery state. Compression of the reservoir 12 may serve to drive fluid out of the reservoir 12, through the delivery sharp(s) 72 and into the target delivery destination in the patient. Additionally, in some embodiments, at least one of an audible or tactile indication may be generated when the delivery sharp(s) 72 are displaced toward the skin.

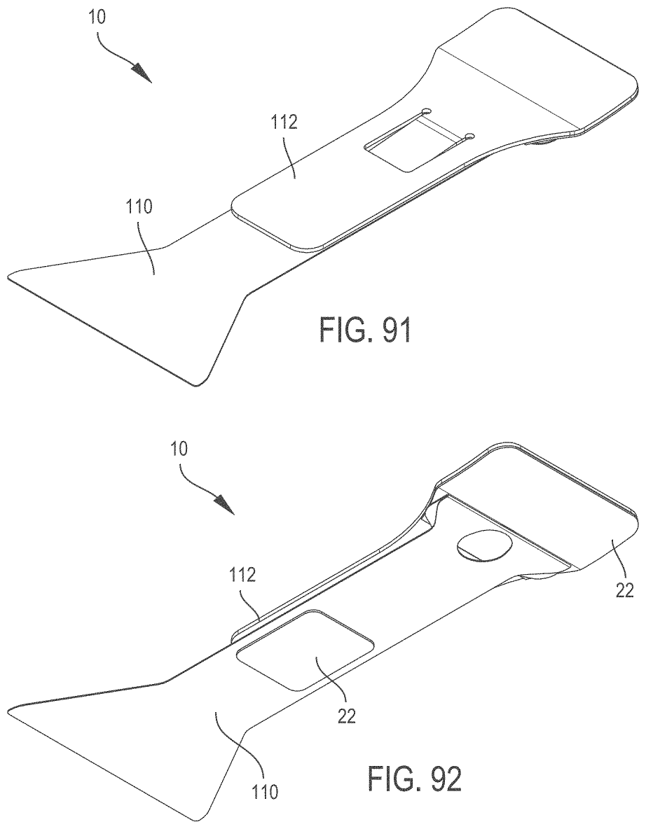

Referring now to FIGS. 91-92, an exemplary delivery device 10 is depicted. As shown, the delivery device 10 may be a thin, low profile assembly which is substantially planar. The delivery device 10 may include a proximal portion 110 and a distal portion 112. The proximal portion 110 may be formed of flexible material and in some embodiments may be elastic so as to allow the proximal portion 110 or at least a portion of the proximal portion 110 to be stretched. The distal portion 112 may be rigid. The proximal face of both the proximal portion 110 and the distal portion 112 may have at least one region which is covered with adhesive 22. The proximal portion 110 may be coupled to the distal portion 112 via the adhesive 22 on the proximal face of the distal portion 112. Such an embodiment may be desirable as the delivery device 10 may be amenable to production via a reel to reel fabrication process in high volume.

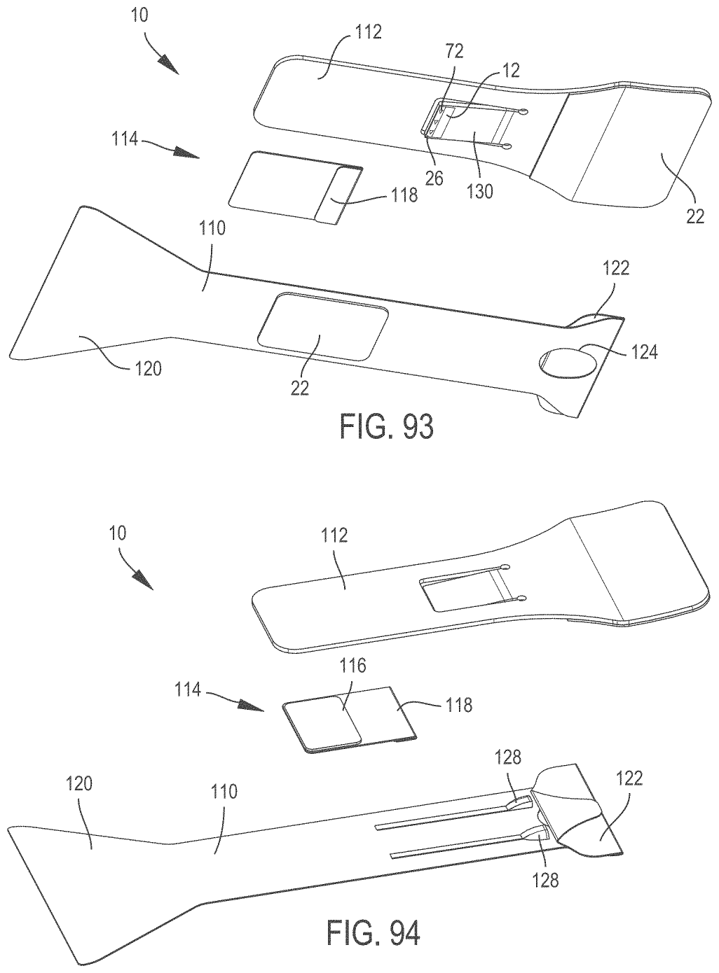

Referring now also to FIGS. 93-94, the proximal portion 110 and the distal portion 112 may also be coupled together via an adhesive locking assembly 114. As shown, the adhesive locking assembly 114 may include a region of locking adhesive 116 which may be disposed on a portion of the proximal face of the distal portion 112. The adhesive locking assembly 114 may include a tether member 118. The tether member 118 may be coupled at a first end to the proximal portion 110 of the delivery device 10 and coupled, at a second opposing end, to the locking adhesive 116 on the distal portion 112. The tether member 118 may be heat staked, welded, or otherwise fixedly coupled to the proximal portion 110 while being relatively lightly coupled to the locking adhesive 116. In some embodiments, the tether member 118 may be constructed of an adhesive liner or backing material which adheres, but is easily peeled from the locking adhesive 116. As shown, the tether member 118 may be at least partially doubled over when the delivery device 10 is in the storage state.

The proximal portion 110 may include a pull tab 120 which may be disposed at a first end of the proximal portion 110. The pull tab 120 may be an enlarged or widened section of the proximal portion 110. In some embodiments, the pull tab 120 may include a roughened surface or may include bumps, ridges, or the like to facilitate grasping. In alternative embodiments, the pull tab 120 may include a cut out so as form a pull ring.

The proximal portion 110 may also include at least one ramp element 128 and a folded region 122 at an end of the proximal portion 110 opposite the pull tab 120. In the example embodiment, the proximal portion 110 includes two ramp elements 128 disposed abreast of one another. The folded region 122 may be folded over upon itself a number of times. In the example, the folded region 122 is folded upon itself twice. Thus, when a pulling force is exerted on the pull tab 120, the folded region 122 may unfurl and spool out proximal portion 110 material to allow the proximal portion 110 to elongate. The at least one ramp element 128 may also be displaced as the folded region 122 feeds out material. The number of folds in the folded region 122 may be adjusted to change the amount that the proximal portion 110 elongates as it is transitioned to the elongated state. The folded region 122 may taper from a larger width to a smaller width over at least a section of the folded region 122. In the example embodiment, the layer of the folded region 122 most proximal to the distal portion 112 tapers to a rounded end. The layer of the folded region 112 most proximal to the distal portion 112 may be substantially immobile and anchored in place by the adhesive 22 of the distal portion 112 as the folded region 122 unfurls.

As best shown in FIG. 93, the delivery device 10 may include a reservoir 12 which may include at least one delivery sharp 72. Any suitable number of delivery sharp(s) 72 may be included in any desired number of rows and/or columns. Any delivery sharp(s) 72 described herein may be used. The delivery sharp(s) 72 may be included on a sharp bearing body 26 which is coupled to the reservoir 12. The reservoir 12 may be disposed on a resilient cantilevered arm 130 defined in the distal portion 112 of the delivery device 10. The folded region 122 of the proximal portion 110 may include a delivery aperture 124. As shown, when the delivery device 10 is in a storage state the delivery aperture 124 may be out of alignment with the delivery sharp(s) 72. Thus, the proximal portion 110 may cover the delivery sharp(s) 72 and block or guard against inadvertent contact with the delivery sharp(s) 72 when the delivery device 10 is in the storage state. The delivery aperture 124 may, however, allow for the delivery sharp(s) 72 of the delivery device 10 to pass through the delivery aperture 124 and access the skin of a user when the delivery device 10 is transitioned to the delivery state.

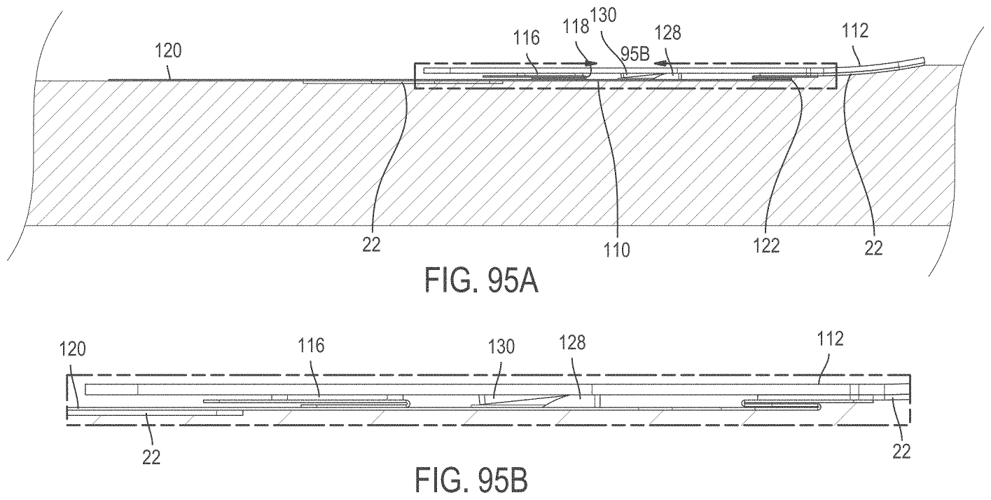

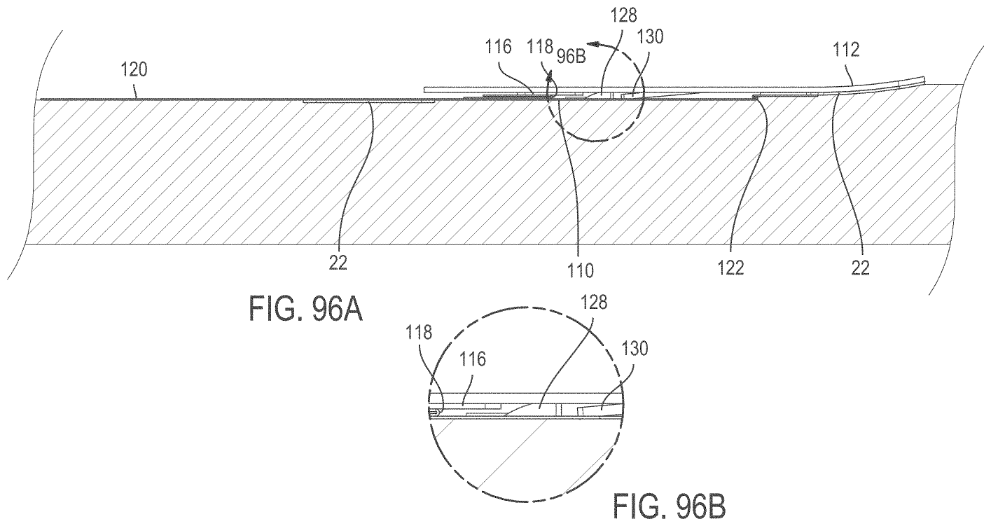

Referring now to FIGS. 95A-96B, to transition the delivery device 10 from the storage state to a delivery state, a pulling force may be exerted on the pull tab 120. The distal portion 112 of the delivery device 10 may be anchored to the skin 44 via the adhesive 22 on the proximal face of the distal portion 112. Thus, the distal portion 112 of the delivery device 10 may be substantially stationary as the transition transpires. The proximal portion 110 may transition from a first state to an elongated state as the delivery device 10 passes from the storage state to the delivery state. As shown, the folded region 122 of the proximal portion 110 may unfurl such that the proximal portion 110 may elongate as the pull tab 120 is pulled. Additionally, in certain embodiments, the proximal portion 110 may stretch allowing for further elongation. The segment of the proximal portion 110 including the adhesive 22 may displace relative to the distal portion 112 of the delivery device 10 as the pull tab 120 is pulled. The adhesive 22 on the proximal portion 110 and distal portion 112 may be displaced apart when the proximal portion 110 is pulled from the first state to the elongated state. As a result, the skin 44 between the adhesive 22 on the proximal portion 110 and the adhesive 22 on the distal portion 112 may become stretched and taut to facilitate puncture.

As the folded region 122 unfurls, proximal portion 110 material may be fed out such that the proximal portion 110 elongates and the delivery aperture 124 is displaced into alignment with the delivery sharp(s) 72. The at least one ramp element 128 may be displaced in the direction of the pull tab 120. The at least one ramp element 128 may keep the cantilevered arm 130 slightly deflected toward the distal portion 112 as the proximal portion elongates. This may keep the delivery sharp(s) 72 of the reservoir from dragging against the proximal portion 110 as the proximal portion 110 is transitioned to the elongated state. As the at least one ramp element 128 is further displaced, the cantilevered arm 130 may ride over a sloped region of the at least one ramp element 128 and be further deflected toward the distal portion 112 of the delivery device 10. As the folded region 122 continues to unfurl, the at least one ramp element 128 may advance past the cantilevered arm 130.

Once the at least one ramp element 128 has cleared the cantilevered arm 130, the cantilevered arm 130 may restore to an undeflected state as shown in FIG. 96A and FIG. 96B. The delivery device 10 may enter the delivery state when the cantilevered arm 130 is clear of the at least one ramp element 128 and restores to its undeflected state. As the cantilevered arm 130 springs back to an undeflected state, the delivery sharp(s) 72 may be displaced through the delivery aperture 124 and may puncture the skin 44. This may establish fluid communication between the delivery sharp(s) 72 and a target delivery destination in the patient. Additionally, the reservoir 12 may be compressed between the skin 44 and the cantilevered arm 130 when the cantilevered arm 130 restores to its undeflected state. This compression may serve to drive fluid out of the reservoir 12 and into the patient via the delivery sharp(s) 72. The compression may also help to ensure that the reservoir 12 is fully emptied during delivery.

As shown, the tether member 118 may be peeled from the lock adhesive 116 as the delivery device 10 transitions from the storage state to the delivery state. Once the delivery device 10 reaches the delivery state, the tether member 118 may be at least partially separated from the lock adhesive 116. The exposed lock adhesive 116 may then adhere to the proximal portion 110 to bond the proximal portion 110 in place. The lock adhesive 116 may aggressively adhere to the proximal portion 110. An attempt to separate the proximal portion 110 and lock adhesive 116 may result in damage to one of the components of the delivery device 10. This may help to ensure that the transition of the delivery device 10 to the delivery state is irreversible. The lock adhesive 116 may also inhibit restoring force exerted by the stretched skin from causing the proximal portion 110 to crumple. Thus, the lock adhesive 116 may hold the adhesive 22 on the proximal portion 110 in place such that the skin remains stretched when the proximal portion 110 is in the elongated state and the user releases the pull tab 120.

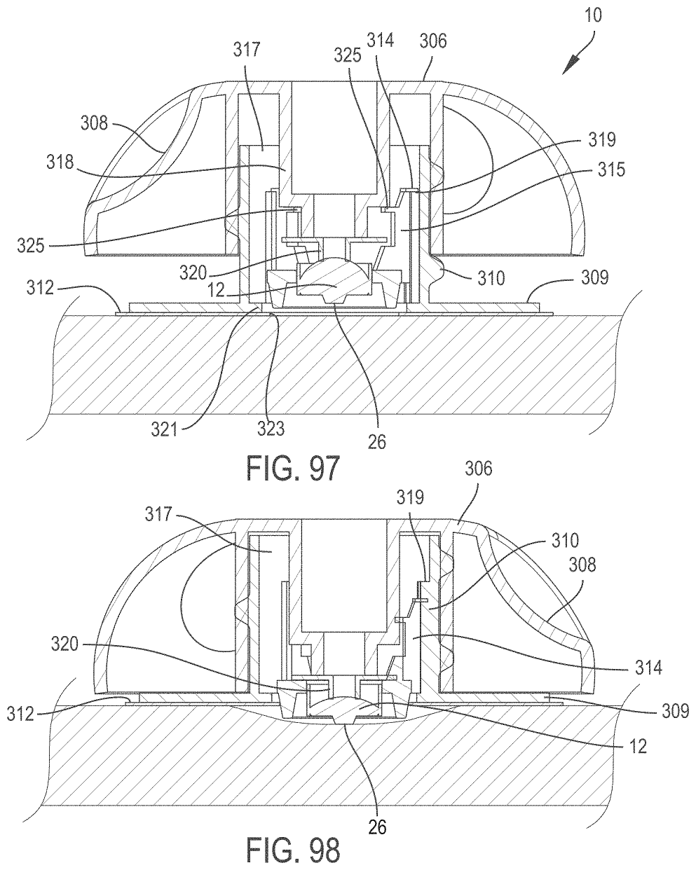

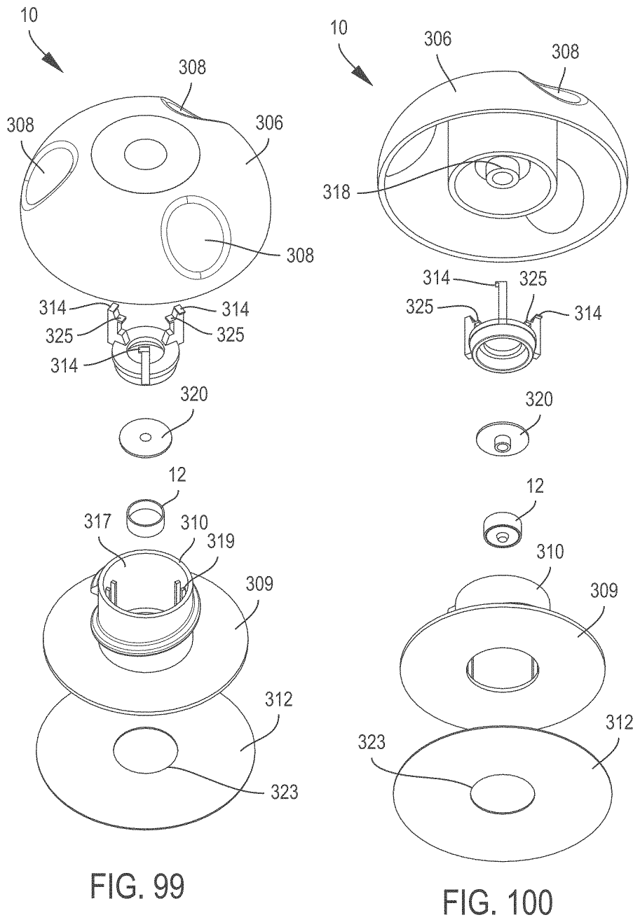

With reference to FIGS. 97-100, another example embodiment of a delivery device 10 is depicted. FIG. 97 depicts the example delivery device 10 in a storage state. FIG. 98 depicts the example delivery device 10 in a delivery state. FIG. 99 and FIG. 100 depict exploded views of the example delivery device 10. As shown, the example delivery device 10 may comprise an actuator. In some embodiments the actuator may form a top 306, or cap, having at least one depression or concavity 308 therein (three such concavities 308 are shown but it would be understood that the number need not be three). The concavities 308 may serve to facilitate twisting of the top 306 by a user via fingertip placement therein. The top 306 may be of a hooded, or convex shape, and may be made of a plastic formed by injection molding or any other suitable technique as would be known to one of skill. One of skill would appreciate that the top 306 need not be limited to any particular shape provided that it can be twisted by a user.

As shown, the exemplary top 306 sits on a base body 309. The top 306 engage with a threaded post or screw 310 included as part of the base body 309. In some non-limiting examples, the threaded screw 310 may be made of a plastic material formed by injection molding and may be formed integrally with the rest of the base body 309. One of skill would appreciate that other materials and manufacturing techniques could be used to construct the threaded screw 310.

In an example embodiment, a user may first remove an adhesive liner 265 (see, e.g., FIG. 24) from the delivery device 10. In addition to covering an adhesive bearing pad 312 of the delivery device 10, the adhesive liner 265 may, in some embodiments, have been attached to cover and maintain a previously sanitized state of the delivery device 10. Such a state may be created prior to attaching the adhesive liner 265 to any of the delivery devices 10 described herein. In some examples, the user may peel off the adhesive liner 265 similarly to the manner in which a user peels a liner off a bandage prior to applying the bandage. Once the liner 265 is removed, a user may then apply the delivery device 10 to the skin. As in the example shown, the adhesive pad 312 may be annular in shape. In some embodiments, the adhesive pad 312 may be ultrasonically welded to the delivery device 10. Those of skill would understand that other suitable techniques to adhere the adhesive pad 312 to the delivery device 10 may be used.

After affixing the delivery device 10 to the skin, the user may then twist the top 306 of the delivery device 10 and thereby cause the top 306 to advance proximally (e.g., toward the skin) along the threads of the threaded screw 310. Inside the threaded screw 310 a breakable material, or frangible 314 may be housed. The frangible 314 may inhibit displacement of the top 306 and other components of the delivery device 10 until a sufficient force is applied to the top 306. This may aid in preventing transition of the delivery device 10 to a delivery state during storage.

In the example embodiment, the frangible 314 is provided as at least one tab which projects from a carriage 315 that may be disposed within the bore 317 of the threaded post 310. In some embodiments, the carriage 315 may include a set of three frangibles 314 which are disposed at even angular increments about a first end of the carriage 315. The bore 317 may include a ledge 319 to support at least one of the frangibles 315 and preferably a ledge 319 to support each frangible 314. With the frangibles 314 resting on the ledge(s) 319, the carriage 315 may be inhibited from displacing within the bore 317 and twisting motion of the top 306 may be impeded. In certain examples, the ledge(s) 319 may each be an end of a track or rail (best shown in FIG. 99) disposed in the bore 317 which may help to guide displacement of the carriage 315 within the bore 315.

The top 306 may incorporate a central projection 318 (e.g. column or stepped column as shown) that rests upon a portion of the carriage 315. As the top 306 screws downward or proximally toward the skin surface, the projection 318 may cause the frangible(s) 314 may be pressed against the respective ledges 319. The pressure exerted against the frangible(s) 314 may cause the frangible(s) 314 to break off, allowing the carriage 315 to travel proximally within the bore 317. The carriage 315 may displace proximally and eventually a second end of carriage 315 (opposite the first end from which the frangibles 314 project) may contact the skin surface. It would be understood by one of skill that once the frangible(s) 314 have broken, reuse of the delivery device 10 may be inhibited.

The adhesive pad 312 of the delivery device 10 may have a central aperture 323 though which a portion of the carriage 315 may extend. Skin may not be retained in position against the delivery device 10 in the region of the central aperture 323. Thus, as the carriage 315 continues to displace proximally, the skin in this region may be pressed and stretched as it is displaced by the carriage 315. This may render the skin aligned with the central aperture 323 taut. The base body 309 may include a shelf 321 which extends into the bore 317 at the proximal end of the bore 317 and acts as a stop surface. The carriage 315 may cease proximal displacement upon contacting the shelf 321.

The top 306 may be at an intermediate point in its travel along the post 310 when the carriage 315 contacts the shelf 321. As shown, the carriage 315 may include a second frangible 325 or set of frangibles 325. In some embodiments, there may be three second frangibles 325 spaced at even angular intervals about the carriage 315. The first frangible(s) 314 may be weaker (e.g. thinner) than the second frangible(s) 325. Thus, the second frangible(s) 325 may only break after the first frangible(s) 314. The projection 318 from the top 306 may abut the second frangible(s) 325 when in the carriage 315 is against the stop provided by the shelf 321. The second frangible(s) 325 may impede displacement of the top 306. Further actuation of the top may exert force against the second frangible(s) 325 and result in breaking of the second frangible(s) 325. With the second frangible(s) 325 broken, the top 306 may be free to move proximally while the carriage 315 remains stationary (against the stop provided by the shelf 321). It would be understood by one of skill that once the frangible(s) 325 have broken, reuse of the delivery device 10 may be inhibited.

As shown, the delivery device 10 may also include a delivery aid 320. The delivery aid 320 may be a flat plate from which a column extends, as shown in the example. The delivery aid 320 may be made of a plastic material formed by injection molding. One of skill would appreciate that other materials and manufacturing techniques could be used to construct the delivery aid 320. The delivery aid 320 may be positioned atop a reservoir 12 that contains a fluid such as, e.g., a medical agent (e.g., a vaccine) and incorporates on an underside a sharp bearing body 26 (see, e.g., FIG. 33) including at least one delivery sharp 72 (see, e.g., FIG. 33).

In some embodiments, the delivery aid 320 may be attached to the proximal end of the projection 318 via adhesive. In some embodiments, the delivery aid 320 may rest upon a shelf within the carriage 315. The reservoir 12 may be retained via friction or via a slip fit within an aperture of the carriage 315 as shown. In some examples, a weak adhesive may hold the reservoir 12 in place within the aperture. In other embodiments, the friction fit may be augmented by a gasket member (e.g. O-ring) disposed between the reservoir 12 sides and the aperture of the carriage 315.

With the second frangible(s) 325 broken, the delivery aid 320 may concentrate force generated as the top 306 is actuated against a reservoir 12 of the delivery device 10. In embodiments where the delivery aid 320 rests upon a shelf in the carriage 315 a portion of the delivery aid 320 may deform or break to allow movement beyond the shelf. The delivery aid 320 and reservoir 12 may move downward as the top 306 continues to advance along the post 310. The force exerted by the top 306 may be sufficient to overcome friction or adhesive holding the reservoir 12 in place. The delivery aid 320 moving downward may cause the reservoir 12 to move downward until the delivery sharp(s) 72 (see, e.g., FIG. 33) penetrate into the skin surface. At that point, as shown in FIG. 98, the reservoir 12 may be sandwiched between the skin and the projection 318 of the top 306. As the top 306 continues to displace proximally, pressure from the delivery aid 320 builds in the reservoir 12, causing delivery of the fluid contained within the reservoir 12 through the delivery sharp(s) 72 and into the patient. At that point the top 306 stops twisting and may have reached the end of its displacement range. The top 306 may abut against the base body 309 at the end of the displacement range and the base body 309 may present a mechanical interference to further displacement. Once the top 306 stops moving, the user may remove the delivery device 10 from the skin.

As previously discussed, it may be desirable to prevent reuse of the delivery device 10. It may also be desirable to provide a delivery device 10 in which the delivery sharp(s) 72 scratch across the surface of the skin prior to penetrating the skin surface. In an embodiment, a delivery device 10 may include an actuation assembly which may include first and second displaceable members. The members may be displaceable from a separated state to a proximal state with relation to each other. The members may transition from the separated state to the proximal state when the delivery device 10 is actuated and/or when the delivery device 10 delivers its contents. The members may include cooperating coupling features that may engage with each other upon the members approaching or reaching the proximal state. Once the cooperating coupling features are engaged, the coupling features may inhibit separation of the members and may maintain the members in the proximal state.

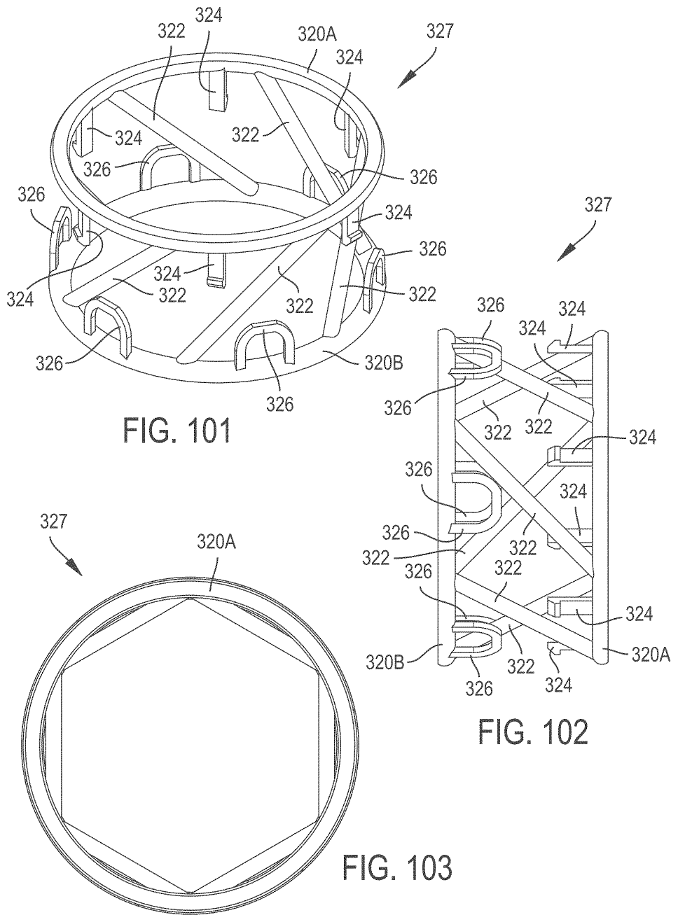

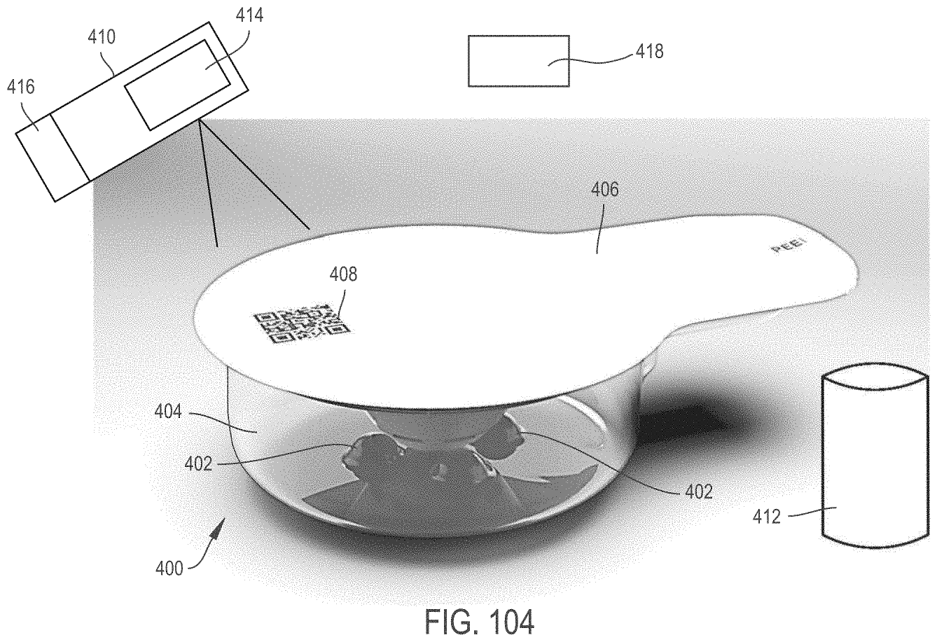

With reference to FIGS. 101-103, an example embodiment of such a portion of an actuation assembly 327 for a delivery device 10 is depicted. The delivery device 10 may comprise a one-piece or monolithically formed flexure. The flexure may be formed as a pair of first and second bodies 320A, 320B, vertically spaced such that the first body 320A is positioned above or in a separate plane than the second body 320B. In the example embodiment, the first and second bodies 320A, B are concentric round bodies, and are specifically shown as circles. The flexure may be made of a bendable plastic formed by injection molding or any other suitable technique as would be known to one of skill. The bodies 320A, 320B may be coupled by at least two flexible struts 322 integral to the flexure. In the example, six such flexible struts 322 are shown but it would be understood that the number need not be six. The struts 322 may be disposed at even angular increments though need not be in all embodiments. The struts 322 may extend between the bodies 320A, 320B at an angle to the bodies 320A, 320B that is not perpendicular.

At least one hook 324, integral to one of the bodies 320A, B may be included. The other of the bodies 320A, B may include at least one catch 326. In the example embodiment, the first body 320A includes a number of hooks 324 which extend downward therefrom toward the second body 320B. Six such hooks 324 are shown but it would be understood that the number need not be six. In the example, the hooks 324 are evenly spaced around the first circle 320A though need not necessarily be in all embodiments. At least one catch 326, integral to the second body 320B is also shown in the example embodiment. Each of the catches 326 is situated at a point on the second body 320B that is not directly beneath a hook 324 of the first body 320A. The example catches 326 extend upward from the second body 320B toward the first body 320A. Six such catches 326 are shown but it would be understood that the number need not be six. The catches 326 may be spaced around the second body 320B at even angular increments. The catches 326 may be positioned such that the respective catches 326 and hooks 324 would engage one another upon actuation of the flexure. The flexure may, for example, be actuated by applying pressure on the flexure via a portion of the delivery device 10 within which it is assembled. The catches 326 may be substantially shaped in the form of an upside down Latin character “U”.

As the first body 320A is displaced toward the second body 320B at least one of the bodies 320A, B may also rotate. If one of the bodies 320A, B is rotationally constrained, only the other of the bodies 320A, B may rotate as the bodies 320A, B are displaced toward one another. With the second body 320B rotationally constrained, pushing down on the first body 320A from above may cause the flexible struts 322 to flex. The hooks 324 may rotationally (about an axis passing through the center points of the bodies 320A, B) displace. The hooks 324 and first body 320A may also translationally displace as the first body 320A approaches the second body 320B. The hooks may also translationally displace with respect to the catches 326 until the hooks 324 contact the catches 326. The hooks 324 may deflect around the catches and then resiliently restore into engagement with the catches 326. Thus, as the struts 322 attempt to resiliently restore to their undeflected state, the first and second bodies 320A, B may be held together by the engagement of the hooks 324 and catches 326. Using a single piece flexure (formed, for example, by injection molding) may allow the delivery device 10 to be manufactured at relatively lower cost. The hooks 324 engagement with the catches 326 may also help to prevent reuse of a delivery device 10 in which the flexure is included. The engaged hooks 324 may also help maintain pressure against a reservoir 12 of a delivery device 10 needed to ensure delivery of a medical agent (e.g., a vaccine) into a patient through one or more delivery sharp 72 (see, e.g., FIG. 33).

The rotational displacement of one of the bodies 320A, B may be harness to help drive the delivery sharp(s) 72 (see, e.g., FIG. 33) across the skin surface to scratch it prior to piercing the skin. In the example embodiment described above, the delivery sharp(s) 72 (see, e.g., FIG. 33) may be constrained to move in tandem with the first body 320A. Thus, the delivery sharp(s) 72 (see, e.g., FIG. 33) would rotate as they are displaced against the skin.

Referring now to FIG. 104, an example package 400 is depicted. The package 400 may house a delivery device 10 during distribution and shipping. The package 400 may also include other components such as printed instructions and/or a medical wipe which may be used for injection site preparation. The package 400 may hold the delivery device 10 and perhaps other contents in place during distribution and may help to prevent premature or inadvertent actuation of the delivery device 10. The package 400 may include an interior cavity (a portion of the package 400 is depicted as transparent in FIG. 104) which may have one or more receivers 402 for a delivery device 10. The receivers 402 may constrain the delivery device 10 within the package 400 such that the delivery device 10 does not shift or jostle excessively during handling. The package 400 may also protect a delivery device 10 from exposure to the surrounding environment. In some embodiments, the package 400 and delivery device 10 may be sterilized (e.g. via EtOx) and the package 400 may maintain the delivery device 10 in this state until just prior to use.

The package 400 itself may be constructed of a first component 404 and a second component 406. The first component 404 may be a rigid base. The rigid base may include a well in which a delivery device 10 (e.g. similar to that shown in FIG. 27) may be housed. The rigid base may be a plastic component. The second component 406 may be a peelable cover which may be coupled to a face of the first component 404. The peelable cover may be removed by a user to access the delivery device 10 just prior to use.

As shown, the package 400 may include at least one unique identifier 408. In other embodiments, a unique identifier 408 may instead or additionally be included on a delivery device 10 or component thereof. Any suitable unique identifier(s) 408 and combinations thereof may be used. In some embodiments, an RFID may be used. In other examples, the unique identifier 408 may be implemented as a printed indicium such as a bar code, data matrix, QR code, etc. The unique identifier 408 may encode various information about the delivery device 10 or contents of the reservoir 12 of the delivery device 10. For example, the unique identifier 408 may include product identity information, product lot information, product serial number, dose size information, etc. An alternative package 401 is depicted in FIGS. 25-26 and may also include a unique identifier 408 as described in relation to FIG. 104.

The unique identifier 408 may be read by a reader 410. The reader 410 may be a dedicated reader or a device such as, e.g., a smart phone, tablet, smart device, laptop, or other portable device in some embodiments. Where a smart phone or the like is utilized, a dedicated delivery device app may run on the smart phone. Where a smart phone or the like is used, the reader 410 may include multiple pieces of hardware which may be used to read a unique identifier 408 (e.g. one or more front facing imager and one or more rear facing imager). The reader 410 used may depend on whether the delivery device 10 is intended for use at home by an individual user or in a clinical setting (e.g. vaccination center, hospital, clinic, or other care facility). A smart phone may be convenient for a use as a reader 410 if delivery via the delivery device 10 is to be, for example, self-administered by a patient (e.g. at home).

The reader 410 may communicate with a database 412 (e.g. via internet, other network, cloud platform, etc.). Prior to use of the delivery device 10, a user may read the unique identifier 408 with the reader 410. The identifier 408 for the delivery device 10 may be checked against the database 412 to ensure the unique identifier 408 is not associated with a delivery device 10 which has already been used, subject to recall, expired, etc. The database 412 may also be updated to indicate that the delivery device 10 associated with the unique identifier 408 has been used. Thus, the reader 410 and database 412 may aid in inventory management. Other usage information may also be saved. In some embodiments, geolocation data indicating the location of the package 400 when the unique identifier 408 is read may also be saved to the database 412.

Depending on the infrastructure available, the data may be stored offline in a memory of the reader 410 until a robust connection to the internet or another suitable network is formed. Thereafter, data may be uploaded to the database 412. Alternatively, data may be sent to the database 412 as it is acquired at the reader 410.

In some embodiments, a patient may be required to pre-register in order to receive a delivery device 10. In some embodiments, the reader 410 may be used to register (e.g. where an app on a smart phone is used). Where the reader 410 uses a smart phone app, the smart phone app may inhibit usage of the reader 410 for the delivery in the event that certain services are not enabled. Such an app may generate a unique identifier or code when predefined requisite services (e.g. location tracking, push notifications) have been enabled. This code may be provided to the database 412 and may be referred to as a registration code. A patient may be required to provide the code in order to receive a delivery device 10. The code may be input to a dispenser or provided to distribution personnel and checked against the database 412. A delivery device 10 may be provided to the patient in the event that the code matches a registration code stored on the database 412. In other embodiments, the controller 416 of the reader 410 may generate a manual input screen to collect desired information in the event that the user elects not to enable one or more service or otherwise provide desired user information. For example, when location tracking is not enabled the app may generate a location data input screen. Input of information into any such screens may be required before a code is generated and provided to the database 412.

In some embodiments, when the unique identifier 408 on the package 400 is read, a controller 416 of the reader 410 may generate instructions on a user interface 414 of the reader 410. The instructions may include text, images, animations, videos, etc. detailing how to use the delivery device 10. The instructions may guide a user, step-by-step, from opening of the package 400 to discarding of a delivery device 10 after use. In some embodiments, each step of the instructions set may be followed by generation, via the controller 416, of a prompt on the user interface 414. The user may be required to interact with the prompt to proceed to the next set of instructions. User interactions may be logged and stored in a database 412. This may aid in confirming that a particular delivery device 10 was not only received by a patient, but also applied and used. In some embodiments, the controller 416 may generate a notification (e.g. visual, tactile, audio, or some combination thereof) in the event that not all steps have been completed. In other embodiments, one or more message may be generated in the event that a user is unresponsive to prompts. For example, where a smart phone or the like is used, the messages may be push notifications generated by the app for the delivery device 10.

In other embodiments, at least one message generator 418 in data communication with the database 412 may generate, for instance, a text message, email, phone call (e.g. automated message or connect the user to a human operator) which may be sent to a telephone number or email address provided by the user. In the event that a delay greater than a predefined period of time has occurred since a previous prompt has been interacted with by the user, the message generator 418 may send a communication to the patient. The type of communication triggered may escalate if no response is received after a communication is sent by a message generator 418. The communication may initially be a text message or push notification. In some embodiments, a push message may preferably be sent or may be sent instead of a text message in the event that cellular service is unacceptable or below a threshold. If no user interaction is received after a predefined escalation period, a message generator 418 may generate a more obtrusive communication (e.g. a phone call). Any suitable number of escalation tiers may be used.

In some embodiments, a patient may also provide additional data via the reader 410. This data may be stored in the database 412 and analyzed (e.g. via a cloud analytics tool or tool set). For example, users may indicate via the reader 410 that a problem with their delivery device 10 has occurred. This data may be checked against data associated with other delivery devices 10 from the same lot. If more than a predetermined threshold of problematic delivery devices 10 are deemed to exist within a lot, the lot may be flagged for investigation and prevented from being distributed or used. Alternatively or additionally, a patient may be prompted to provide certain post injection information via the reader 410. For example, the patient may be requested to fill out a side-effect questionnaire or other form which may be generated by the controller 416 of the reader 410 on the user interface 414. Side-effect data may be analyzed to identify patterns common to certain patient types or delivery devices 10 (e.g. delivery devices 10 of the same lot or holding the same contents). Analysis may be conducted via a cloud analytics tool or toolset.

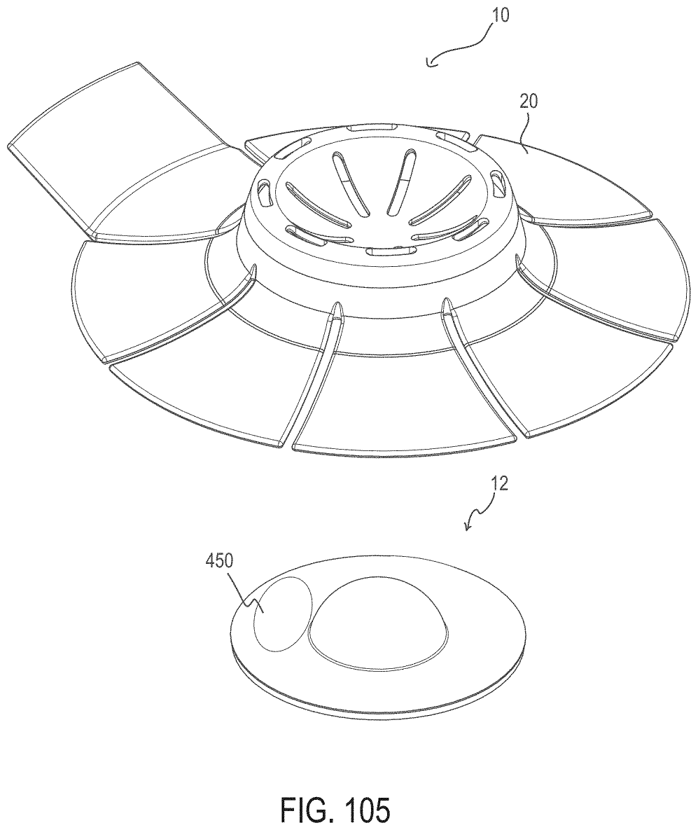

In certain examples and referring now also to FIG. 105, a delivery device 10 may include a revealing indicator 450 or identifier which may be hidden when the delivery device 10 is in a storage state. Upon or after use of such a delivery device 10, the revealing indicator 450 may become accessible. The revealing indicator 450 may then be scanned by a reader 410 and confirmation of the scan or scan data captured during the scan may be sent to the database 412. The revealing indicator 450 may encode a unique identifier (e.g. barcode, QR code, data matrix, etc.) specific to the delivery device 10 in certain examples. Thus, the revealing indicator 450 may serve as a confirmation that a delivery was performed with a specific delivery device 10. In some examples, the database 412 or certain data within the database 412 may also be accessible via a payment provider (e.g. governmental body, insurer, etc.). In order to help ensure that the revealing identifier 450 is scanned to document delivery via a delivery device 10, reimbursement or payment may be tied to scanning of the revealing identifier 450. For example, a payment service may query the database 412 to determine usage status of a delivery device 10 and only allow payment in the event that a delivery device 10 has been used. Indication in the database 412 that the revealing indicator 450 has been scanned may flag a delivery device 10 as used.

Referring now to FIG. 105, in some examples, a delivery device 10 may include a first portion and a second portion which may become disassociated when a user removes the delivery device 10 from the skin. In the example embodiment, a delivery device 10 including a main body 20 and a reservoir assembly 12 similar to that shown in FIG. 27 for instance is depicted. A revealing indicator 450 may also be included in other delivery device 10 embodiments described herein. In the example illustrated in FIG. 105, the exemplary first portion is the reservoir assembly 12 (see, e.g., FIG. 59) and the exemplary second portion is the main body 20. In such an example, a revealing indicator 450 may be placed on a distal side of the reservoir assembly 12. View of the revealing indicator 450 may be obstructed by the main body 20 when the first and second portion are coupled together (the main body 20 may be opaque or at least sufficiently translucent). As shown, when the main body 20 and reservoir assembly 12 become disassociated, the revealing indicator 450 may become visible and may be scanned to aid in confirming that that delivery device 10 has been used.

In some examples, the holder 270 (see, e.g., FIGS. 53A-53D and FIGS. 54A-54C) of the reservoir assembly 12 may include an adhesive on at least a portion of the proximal face of the holder 270. In various embodiments where the of the holder 270 reservoir assembly 12 includes tabs 277 (see, e.g., FIG. 54B) which couple into slits of the main body 20, the tabs 277 may disengage with the slits 278 (see, e.g., FIG. 47) of the main body 20 when the user attempts to remove the delivery device 10 from the skin. The bond between the adhesive on the holder 270 and the skin may be sufficient to overcome or disengage the coupling between the tabs 277 and the main body 20 when the patient pulls on the main body 20 to remove the delivery device 10. That is, the adhesive may withstand any forces applied to disassociate the main body 20 from the rest of the delivery device 10 when a user pulls on the main body 20. Thus, the reservoir portion 12 may remain adhered to the skin and the main body 20 may be removed. The revealing indicator 450 included on the reservoir 12 may become visible and may be scanned by a reader 410 (see, e.g., FIG. 104). The reservoir assembly 12 may then be peeled off the skin by the patient. In examples where the reservoir assembly 12 is adhered to the main body 20, an adhesive connection between the skin and reservoir assembly 12 may be stronger than the adhesive connection between the reservoir assembly 12 and main body 20. Thus, as a patient pulls on the main body 20, the main body 20 may decouple from the reservoir assembly 12 to reveal the revealing indicator 450.

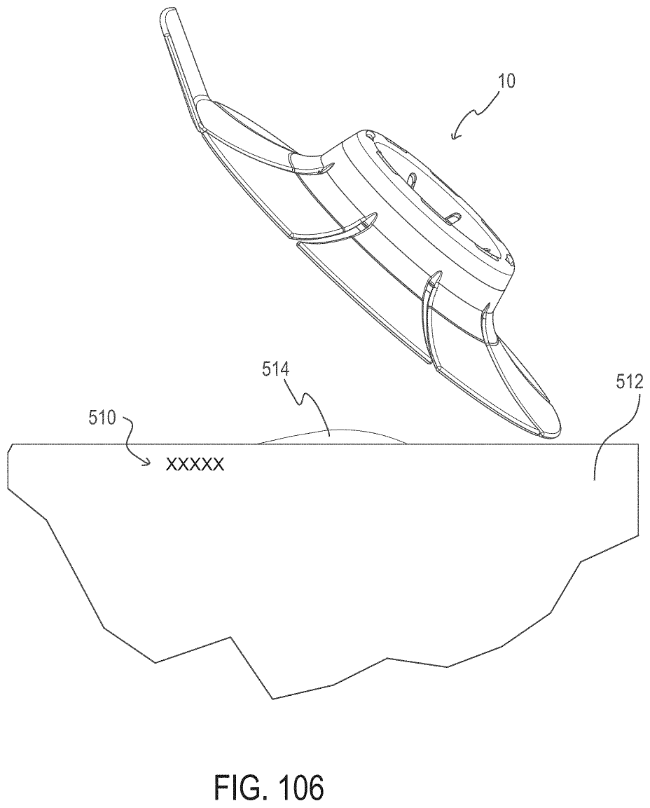

In other embodiments, and referring now to FIG. 106, a delivery device 10 may provide a mark 510 on the skin when the delivery device 10 is applied or applied for at least a predetermined period of time. In some embodiments, a marking agent such as an ink or the like may be included on a portion of the delivery device 10 adjacent the skin 512. In certain examples, a rocker member 526 (see, e.g., FIGS. 63A-64B) may, for example, bear a marking agent. Alternatively, the marking agent may be manufactured into a skin compatible adhesive for coupling the delivery device 10 to the skin during use. In examples where pressure sensitive adhesive is used, pressure applied when the delivery device 10 is used may activate the adhesive and also release the marking agent. The marking agent may at least partially transfer to the skin 512 or otherwise mark the skin 512 when the delivery device 10 is applied. Alternatively, the delivery device 10 may apply a temporary tattoo over the course of the injection. In still other embodiments, no marking agent may be included and the mark 510 may be a temporary impression left on the skin due to a portion of a delivery device 10 pressing against the skin during delivery (further described in relation to FIGS. 63A-64B). In some embodiments, the mark 510 created may have a pattern which encodes certain information about the delivery device 10. In the example embodiment, a series of “X”'s are shown though any suitable mark 510 may be produced. As indicated by the injection bleb 514, the mark 510 may become visible after the injection is completed and the delivery device 10 is removed from the skin 512.

When the delivery device 10 is removed, the mark 510 left on the skin 512 may be imaged by a reader 410 (see, e.g., FIG. 104). This mark 510 may help to verify that an injection has been administered to a patient by a delivery device 10. In some embodiments, the controller 416 (see, e.g., FIG. 104) of the reader 410 may analyze the image to determine whether the mark 510 is present. When the controller 416 determines an appropriate mark 510 is present, the database 412 (see, e.g., FIG. 104) may then be updated to indicate that the delivery device 10 associated with the previously scanned unique identifier 408 has been used. Additionally, the mark 510 may be of a known size and may be in a known position relative to other components (e.g. any delivery sharp(s) 72 of a delivery device 10. Thus, the mark 510 may serve as a fiducial reference which may facilitate various image analysis via a controller 416. For example, the mark 510 may be used to ensure other identified features in an image (e.g. an injection bleb) correspond to an expected range of positions and/or sizes using the mark 510 as a reference. It should be understood that in embodiments mentioned herein where the controller 416 is described as performing image analysis or other analysis, this need not be the case. For example, the image may be communicated to the database 412 by the reader 410 and a cloud analytics tool may be utilized to verify that the image indicates a delivery has occurred. Regardless of where analysis is performed, the image may be uploaded to the database 412.

Referring now primarily to FIG. 107, in certain embodiments the reader 410 (see, e.g., FIG. 104) may include at least one image sensor which is sensitive to one or more wavelength(s) outside of the visible spectrum. The non-visible spectrum wavelength or spectrum to which the image sensor is sensitive may be a wavelength which has a higher penetration depth into skin than light in the visible spectrum. The reader 410 may include at least one image sensor which is sensitive to various wavelengths in the infrared spectrum (e.g. near infrared). A CCD or CMOS image sensor may be included in various embodiments. Any such capable sensor in the reader 410 may not include an IR filter (e.g. IR blocking film) which is commonly applied to typical consumer imaging equipment. The sensor may be associated with a filter which blocks visible light. In some embodiments, the imager may be a thermographic or thermal imaging imager. Multiple imagers which capture images in different non-visible spectrums may be included (e.g. at least one for near infrared and at least one for longer infrared wavelengths).

After a delivery device 10 has been used, the reader 410 may be used to capture at least one image of the injection site. At least one image may be captured or generated based on light other than that in the visible spectrum. Image data in the visible spectrum may also be captured in some embodiments. A controller 416 (see, e.g., FIG. 104) of the reader 410 (see, e.g., FIG. 104) may generate a prompt (e.g. within an app) to capture the image(s). In certain embodiments, the controller 416 may also automatically open an image capture program. The controller 416 may enable image capture with an appropriate imager of the reader 410 (e.g. if multiple imagers are included in a reader 410).

The image data may be analyzed to determine the presence of a bleb 514 formed within the skin during the delivery. Analysis may be automated or may be conducted by a human operator viewing images via a network connection to a database 412 (see, e.g., FIG. 104). As light outside of the visible spectrum may have greater penetration into the skin, the use of such light for imaging purposes may make certain subsurface features in the skin discernable or more easily discernable. This may help to, for example, facilitate detection of the bleb 514. Additionally, the injected agent may differ in temperature from the patient. An area with a temperature that differs from the surrounding area on the patient may be discernable. The injection site may, for example, be cooler than the surrounding regions of the patient. FIG. 107, for instance, depicts a thermal image of an arm after injection. As shown, a cool region (darker gray) is discernable in the image and corresponds to the location of a bleb 514 on the skin.

In the event that the image(s) contain characteristics of a bleb 514, it may be concluded that a delivery was actually performed with the delivery device 10 and was successful. In some embodiments, the image(s) may be required to comply with at least one predefined characteristic of interest. For example, in certain implementations, it may be required that a bleb 514 is detected and is of a certain size (e.g. in relation to a marking 510). Additionally, the image(s) may be required to be devoid of characteristics indicative of an improper injection. For example, where a thermal imager is used, a cool region corresponding to a bleb 514 with one or more adjacent cool region or a cool region 514 or a size beyond a certain limit may be flagged as having characteristics of a leak. In such an example, the analysis may indicate an unsuccessful delivery from a delivery device 10.

The analysis may be performed by the controller 416 (see, e.g., FIG. 104) of the reader 410 (see, e.g., FIG. 104). Alternatively, the analysis may be conducted on a networked server such as a cloud server. As mentioned above, human analysis may be used. The outcome of the analysis and optionally the image(s) may be provided to and stored on at least one database 412 (see, e.g., FIG. 104). In the event that the image(s) indicate an improper delivery or no delivery, a notification for the user may be generated for display on the reader 410 (e.g. by the controller 416 see, e.g., FIG. 104). In the event that a proper delivery has been documented, a confirmation may be generated that the injection was successful.

In examples where the reader 410 is a smart phone, any app used may generate a confirmation that injection with a delivery device 10 was performed upon request by a user. The controller 416 of the reader 410 may generate an option (e.g. displayed button) which may be interacted with by the user to display a conformation notice regarding the injection. Where a delivery device 10 is used to perform a vaccination, the app may provide a proof of vaccination or virtual vaccination record or card which is automatically populated with various information about the user's vaccination. The vaccination record may be stored in a memory of the reader 410 or may be stored in a database 412 (see, e.g., FIG. 104) accessible via the reader 410 (or stored in multiple locations). Thus, the reader 410 may be used to provide proof of injection if necessary. For example, the reader 410 could be used to prove vaccination in order to access certain spaces (restaurants, stadiums, workplaces, other venues, airplanes or airports, ships, public transportation, etc.).

In still other embodiments, a container 350 which houses a packet 208 may be included in a delivery device 10 as described elsewhere herein (see, e.g., FIG. 11). One of the packet 208 and container 250 may include a first chemical or chemicals while the other may include a second chemical or chemical. A dye or dyes may also be included in one of the container 350 and packet 208. When pressure is applied to the delivery device 10 to shift the delivery device from the storage state to the delivery state, the packet 208 may rupture and the first and second chemicals may mix. The chemicals may react and produce a visually perceptible effect. A chemiluminescent reaction may, for example, be initiated when the first and second chemicals are mixed. In examples of such embodiments, chemicals commonly used in glow sticks may, for instance, be filled into the container 350 and packet 208.

The delivery device 10 may include one or more window (e.g. slots 254 or apertures 255 in a main body 20 like the example shown in FIGS. 21A-21I) through which light generated by the reaction may be perceived. A reader 410 (see, e.g., FIG. 104) may image the delivery device 10 as it is injecting and a controller 416 (see, e.g., FIG. 104) may analyze the image(s) to verify presence of the light from the reaction. When the controller 416 determines that a chemiluminescent reaction is documented in the image(s), the controller 416 may communicate with the database 412 and the database 412 may be updated to indicate the delivery device 10 has been used.

In some alternative embodiments where two (or more) chemicals are combined to produce a visibly perceptible effect, the first chemical or chemicals may be included in the medical wipe. The user may wipe the injection site during preparation and some of the first chemical or chemicals may be deposited on the skin surface. The second chemical or chemicals may be carried by or released by the delivery device 10 during the course of the injection. The first and second chemicals may interact by the time the injection has completed and may, for instance, result in a color change to a delivery indicating color. An image of the injection site may be taken by the reader 410 and analyzed (e.g. locally by the controller 416 or via a cloud analytic tool after the image is uploaded to the database 412) to confirm presence of the delivery indicating color. In the event that the color change is documented in the image, the database 412 may be updated to indicate that the delivery device 10 has been used.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

What is claimed is:

1. A delivery device for delivery of medical agent to a biological barrier comprising:
A main body including a central region defining a receptacle, a peripheral region defined by a number of petal members, and a set of retention tabs extending into the receptacle;
A reservoir including at least one delivery sharp, a main interior volume and at least one septum in fluid communication with the main interior volume, the reservoir coupled to the main body via the retention tabs;
An adhesive coupled to the main body; and
At least one bias member positioned within the receptacle between the reservoir and a wall of the receptacle.

2. The device of claim 1, wherein the bias member is configured to distort upon transition of the main body from a first state to a second state, the bias member configured to restore against the reservoir urging fluid out of the at least one delivery sharp once distorted by transition of the main body from the first to the second state, the reservoir configured to displace toward the biological barrier as the main body transitions from the first state to the second state.

3. The device of claim 1, wherein the main interior volume is formed partially by a wall including at least one collapse facilitator.

4. The device of claim 1, wherein the at least one bias member is formed of an elastomeric material and includes at least one hollow portion.

5. The device of claim 1, wherein the bias member formed of an elastomeric material and has at least a first and second tier having footprints with differing areas.

6. The device of claim 1, wherein the reservoir comprises a guard defined at least partially by a septum, the reservoir further comprising a fluid pathway from the main interior volume to the septum.

7. The device of claim 1, wherein the device further comprises a septum including a plug portion seated in fluid tight relationship within an aperture through a rigid portion of the reservoir.

8. The device of claim 1, wherein the reservoir includes a rigid portion and a flexible portion coupled to the rigid portion, and the device further comprises a septum including a first section disposed within a passage through the rigid portion, the septum including a second section having recessed channel and being configured to block displacement of the flexible portion into the recessed channel.

9. The device of claim 1, wherein the reservoir is filled with an agent from a list consisting of an overdose intervention drug, at least one vaccine, and a coronavirus vaccine.

10. The device of claim 1, wherein the main body is formed integrally with the retention tabs in a side action free injection molding operation.

11. A method of delivering an agent comprising:
affixing to a surface a delivery device including at least one delivery sharp coupled to a collapsible reservoir containing the agent;
applying pressure on the delivery device to transition first and second portions of the delivery device to respective distorted states, penetrate the surface with the at least one delivery sharp, and initiate collapse of the reservoir to force the agent out of the at least one delivery sharp; and
relieving pressure on the delivery device to allow the first portion of the delivery device to at least partially restore from its distorted state and lift the surface.

12. The method of claim 11, wherein the transitioning the first portion to the distorted state comprises spreadingly displacing segments of the first portion with respect to one another.

13. The method of claim 11, wherein the method further comprises contacting the surface with a protrusion from the reservoir after the delivery sharp has penetrated into the surface.

14. The method of claim 13, wherein the method further comprises creating a mark on the surface with the protrusion.

15. The method of claim 11, wherein the collapse of the reservoir continues while the first portion of the delivery device at least partially restores from its distorted state.

16. The method of claim 11, wherein the at least one delivery sharp is a spaced array of microneedles.

17. The method of claim 11, wherein transitioning the second portion to its distorted state comprises transitioning the second portion from a protruding state to an indented state and compressing a bias member against the reservoir.

18. A delivery device for delivering fluid into a biological barrier comprising:
a main body including a peripheral region with a plurality of petal members; and
a reservoir coupled to the main body including at least one delivery sharp; and
wherein the main body is configured to transition between a first state and a second state, the petal members configured to distort from an initial state, to an intermediate state, and at least partially restore from the intermediate state as the main body transitions from the first state to the second state.

19. The device of claim 18, wherein the reservoir includes a rigid portion having at least one protuberance configured to contact the barrier prior to the majority of the rigid portion as the reservoir displaces along the displacement path.

20. The device of claim 18, wherein the reservoir is at least partially collapsible.

21. The device of claim 18, wherein the petal members are at least partially covered with adhesive and are configured to spreadingly displace as the petal members distort from the initial state to the intermediate state.

22. The device of claim 18, wherein the majority of each petal member is substantially flat.

23. The device of claim 18, wherein the majority of each petal member extends at a constant angle to a center axis of the delivery device and each petal member includes a segment having a curvature leading to a central region of the main body.

24. The device of claim 18, wherein each petal member includes a curved segment intermediate a first substantially flat region and second substantially flat region of each petal member.

25. The device of claim 18, wherein the peripheral edge of the delivery device is defined by the outermost edges of the petal members, the outermost edges being curved along a path other than that defined by a constant radius extending from a point along the center axis of the delivery device.

26. The device of claim 25, wherein the main body includes a central region formed integrally with the peripheral region, the central region including a portion configured to flip from a protruding state to a depressed state as the delivery device transitions from the first state to the second state, and wherein the delivery device further comprises a bias member between the reservoir and the portion, the bias member configured to distort when the portion is flipped to the depressed state.
